(19) United States
(12) Patent Application Publication
(10) Pub. No.: US 2022/0016152 A1
(43) Pub. Date: Jan. 20, 2022

(54) IMMUNE MODULATORY COMBINATIONS AND METHODS FOR TREATING CANCERS

(71) Applicant: Birdie Biopharmaceuticals, Inc., Grand Cayman (KY)

(72) Inventors: Lixin Li, Edison, NJ (US); Walter Lau, Edison, NJ (US)

(21) Appl. No.: 17/417,357

(22) PCT Filed: Dec. 17, 2019
(86) PCT No.: PCT/US2019/066796

§ 371 Date: Jun. 22, 2021

Related U.S. Application Data
(60) Provisional application No. 62/785,137, filed on Dec. 26, 2018.

(51) Int. Cl. A61K 31/7068; A61K 31/437; A61K 39/395; A61P 35/00

(52) U.S. Cl. CPC A61K 31/7068; A61P 35/00; A61K 39/3955; A61K 31/437

(57) ABSTRACT

The presently disclosed embodiments relate to immune modulatory compositions and methods for treating cancers using combination therapy.

### CROSS REFERENCE TO RELATED APPLICATIONS

This application claims the benefit of U.S. Provisional Patent Application No. 62/785,137, filed on Dec. 26, 2018, the entire contents of which are incorporated by reference herein.

### FIELD OF THE INVENTION

The presently disclosed embodiments relate to immune modulatory combinations and methods for treating cancers using combination therapy.

### BACKGROUND OF THE INVENTION

Myeloid-derived suppressor cells (MDSC) are a diverse population of immature myeloid cells that have potent immune-suppressive activity and are increasingly recognized as a major spoiler of antitumor immunity because they accumulate in virtually all individuals with cancer. They encompass a range of myeloid cells that are developmentally immature and in different stages of myelopoiesis.

There are two major subtypes of MDSC in mice, monocytic MDSC (M-MDSC) and granulocytic MDSC (G-MDSC). M-MDSCs are mononuclear and G-MDSCs are polymorphonuclear. Both types express the myeloid lineage marker CD11b and the granulocytic marker Gr1. Gr1 includes two distinct molecules, Ly6C and Ly6G. M-MDSC have a lower level of expression of Gr1 and express Ly6C, whereas G-MDSC have higher levels of Gr1 and express Ly6G.

There are also two types of human MD SC. Both types express CD11b; however, there is no equivalent to the mouse Gr1 marker. Instead, human M-MDSCs are characterized by their expression of CD14 and G-MDSC by their expression of CD15 and CD66b. Both types also express the general myeloid maker CD33 and lack linage markers for lymphocytes and NK cells. Because these markers are also expressed by monocytes, MDSC are distinguished from monocytes by their absence of HLA-DR.

Studies in both mice and humans have demonstrated that MDSC accumulate in most individuals with cancer, where they promote tumor progression, inhibit antitumor immunity, and are an obstacle to many cancer immunotherapies. For example, MDSC promote tumor growth by facilitating neovascularization through their production of VEGF, and by driving invasion and metastasis through their production of matrix metalloproteases; by upregulating Arg1 and inducible NO synthase, MDSC prevent T cell activation and function. In additional, MDSC downregulate macrophage productions of the type 1 cytokine IL-12, inhibit NK-mediated tumor cell lysis, and induce T regulatory cells.

Toll-like receptors (TLRs) are a crucial part of the innate immunity and present the first line of defense against pathogens. Resiquimod is the ligand for TLR7 and 8 and directly activates innate immune cells, including myeloid dendritic cells, plasmacytoid dendritic cells, and monocytes/macrophages. This activation may result in activation of co-stimulatory molecules, production of antiviral cytokines, and stimulation of cell-mediated NK and T cell immune responses.

### SUMMARY OF THE INVENTION

In general, the presently disclosed embodiments provide therapeutic combinations and methods for treatment of cancers.

In one aspect, the presently disclosed embodiments provide a method for treating tumor or abnormal cell proliferation, in a subject that needs such treatment, comprising administering to said subject:

a chemotherapeutic that is of an amount that is capable of reducing the myeloid-derived suppressor cell (MDSC) population in blood, spleen, and/or tumor microenvironment in said subject; and an effective amount of an immunotherapeutic.

In some embodiments, the chemotherapeutic comprises an MDCS inhibitor.

In some embodiments, the MDSC inhibitor is a molecule that causes the induction of MDSC apoptosis and/or necrosis, or cytotoxicity, or the inhibition of c-kit, or VEGFR, or ARG1, or iNOS, or S100 or MMPs functions of MDSCs, or ROS ERK activation or antioxidant genes. In some embodiments, the inhibitor is selected from the group consisting of: Paclitaxel, Gemcitabine, 5-Fluorouracile, Oxaliplatin, Cisplatin, Carboplatin, Dasatinib, Sunitinib, and Doxorubicin.

In some embodiments, the chemotherapeutic is provided in an amount that is capable of reducing the amount of MDSC in blood, spleen and/or tumor microenvironment by 10% to 95% in said subject, preferably by at least 30%.

In some embodiments, the chemotherapeutic is provided in an amount that is less than the amount when used as a monotherapy, such as is 50% of standard monotherapic dose.

In some embodiments, the chemotherapeutic is gemicitabine and the amount is 400-625 mg/m².

In some embodiments, the MDSC expresses CD11b, CD15, CD33 and CD66b. In some embodiments, the MDSC expresses CD11b, CD14, and CD33.

In some embodiments, the chemotherapeutic is administrated prior to the administration of said immunotherapeutic and is within 7 days prior to the administration of said immunotherapeutic.

In some embodiments, the chemotherapeutic is administrated prior to the administration of said immunotherapeutic.

In some embodiments, the chemotherapeutic is administrated at least one, two, three, four, five days, six days, or seven days prior to the administration of said immunotherapeutic.

In some embodiments, the immunotherapeutic is administrated after the amount of MDCS in blood, and/or tumor microenvironment is reduced by 10% to 95%, or at least about 50%, in said subject after said administration of said chemotherapeutic.

In some embodiments, the immunotherapeutic comprises a TLR7 agonist but not a TLR8 agonist, a TLR8 agonist but not a TLR7 agonist, or an agonist for both TRL7 and TLR8.

In some embodiments, the immunotherapeutic is an agonist for both TLR7 and TLR8.

In some embodiments, the immunotherapeutic has a structure of Formula (I):

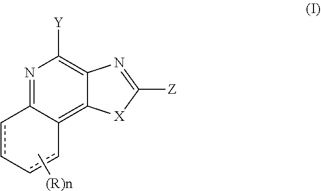

wherein dashed line represents bond or absence of bond;
X is S or —NR₁, R₁ is —WO—W₁—W₂—W₃'W₄,
WOis a bond, alkyl, alkenyl, alkynyl, alkoxy, or -alkyl-S-alkyl-,
W₁ is a bond, —O—, or NR₂—, wherein R₂ is hydrogen, alkyl or alkenyl,
W₂ is a bond, —O—, —C(O)—, —C(S)—, or S(O)₂—,
W₃ is a bond, —NR₃—, wherein R₃ is hydrogen, alkyl or alkenyl,
W₄ is hydrogen, alkyl, alkenyl, alkynyl, alkoxy, cycloalkyl, aryl, aryloxy, heteroaryl, or heterocyclyl, each of which is optionally substituted by one or more substituents selected from the group consisting of hydroxyl, alkoxy, alkyl, alkenyl, alkynyl, cycloalkyl, aryl, heteroaryl, heterocyclyl, —NH₂, nitro, -alkyl-hydroxyl, -alkyl-aryl, -alkyl-heteroaryl, -alkyl-heterocyclyl, —O—R₄, —O-alkyl-R₄, -alkyl-O—R₄, —C(O)—R₄, -alkyl-C(O)—R₄, -alkyl-C(O)—O—R₄, —C(O)—O—R₄, —S—R ₄, —S(O)₂—R₄, —NH—S(O)₂—R₄, -alkyl-S—R ₄, -alkyl-S(O)₂—R₄, —NHR₄, —NR₄R₄, —NH-alkyl-R₄, halogen, —CN, —NO₂, and —SH, wherein R₄ is independently hydrogen, alkyl, alkenyl, -alkyl-hydroxyl, aryl, heteroaryl, heterocyclyl, or haloalkyl;
Z is hydrogen, alkyl, alkenyl, alkynyl, alkoxy, aryl, haloalkyl, heteroaryl, heterocyclyl, each of which can be optionally substituted by one or more substituents selected from the group consisting of hydroxyl, alkoxy, alkyl, alkenyl, alkynyl, aryl, heteroaryl, heterocyclyl, halogen, cyano, nitro, —N(R₅)₂, -alkoxy-alkyl, -alkoxy-alkenyl, —C(O)-alkyl, —C(O)—O-alkyl, —O—C(O)-alkyl, —C(O)—N(R₅)2, aryl, heteroaryl, —CO-aryl, and —CO-heteroaryl, wherein each R₅ is independently hydrogen, alkyl, haloalkyl, -alkyl-aryl, or -alkyl-heteroaryl;
R is hydrogen, alkyl, alkoxy, haloalkyl, halogen, aryl, heteroaryl, heterocyclyl, each of which is optionally substituted by one or more substituents selected from the group consisting of hydroxyl, alkoxy, alkyl, alkenyl, alkynyl, cycloalkyl, aryl, heteroaryl, heterocyclyl, —NH₂, nitro, -alkyl-hydroxyl, -alkyl-aryl, -alkyl-heteroaryl, -alkyl-heterocyclyl, —O—R₄, —O-alkyl-R₄, -alkyl-O—R₄, —C(O)—R₄, —C(O)—NH—R₄, —C(O)—NR₄R₄, -alkyl-C(O)—R₄, -alkyl-C(O)—O—R₄, —C(O)—O—R₄, —O—C(O)—R₄, —S—R ₄, —S(O)₂—R₄, —NH—S(O)₂—R₄, -alkyl-S—R ₄, -alkyl-S(O)₂—R₄, —NHR₄, —NR₄R₄, —NH-alkyl-R₄, halogen, —CN, and —SH, wherein R₄ is independently hydrogen, alkyl, alkenyl, alkoxy, -alkyl-hydroxyl, aryl, heteroaryl, heterocyclyl, or haloalkyl;
n is 0, 1, 2, 3, or 4;
Y is —NR₆R₇, —CR₆R₇R₈, or -alkyl-NH₂, each of which can be optionally substituted by one or more substituents selected from the group consisting of hydroxyl, alkoxy, alkyl, alkenyl, alkynyl, —NH₂, halogen, —N(R₅)₂, -alkoxy-alkyl, -alkoxy-alkenyl, —C(O)-alkyl, —C(O)—O-alkyl, —C(O)—N(R₅)₂, aryl, heteroaryl, —CO-aryl, and —CO-heteroaryl,
wherein R₆, R₇ and R₈ are independently hydrogen, alkyl, alkenyl, alkoxy, alkylamino, dialkylamino, alkylthio, arylthio, -alkyl-hydroxyl, -alkyl-C(O)—O—R₉, -alkyl-C(O)—R₉, or -alkyl-O—C(O)—R₉, wherein each R₅ is independently hydrogen, alkyl, haloalkyl, -alkyl-aryl, or -alkyl-heteroaryl, wherein R₉ is hydrogen, alkyl, alkenyl, halogen, or haloalkyl;
X and Z taken together may optionally form a (5-9)-membered ring.

In some embodiments, the immunotherapeutic is a compound selected from the group consisting of: 2-propylthiazolo[4,5-c]quinolin-4-amine, 1-(2-methylpropyl)-1H-imidazo[4,5-c]quinolin-4-amine, 4-amino-2-(ethoxymethyl)-a,a-di-methyl-1H-imidazo[4,5-c]quinoline-1-ethanol, 1-(4-amino-2-ethylaminomethylimidazo-[4,5-c]quinolin-1-yl)-2-methylpropan-2-ol, N-[4-(4-amino-2-ethyl-1H-imidazo[4,5-c]quinolin-1-yl)butyl-]methanesulfonamide, 4-amino-2-ethoxymethyl-aa-dimethyl-6,7,8,9-tetrahydro-1h-imidazo[4,5-c]quinoline-1-ethanol, 4-amino-aa-dimethyl-2-methoxyethyl-1h-imidazo[4,5-c]quinoline-1-ethanol, 1-{2-[3-(benzyloxy)propoxy]ethyl}-2-(ethoxymethyl)-1H-imidazo[4,5-c]quinolin-4-amine, N-[4-(4-amino-2-butyl-1H-imidazo[4,5-c][1,5]naphthyridin-1-yl)butyl]-n′-butylurea, N1-[2-(4-amino-2-butyl-1H-imidazo[4,5-c][1,5]naphthyridin-1-ypethyl]-2-amino-4-methylpentanamide, N-(2-{2-[4-amino-2-(2-methoxyethyl)-1H-imidazo[4,5-c]quinolin-1-yl]ethoxy}ethyl)-n′-phenylurea, 1-(2-amino-2-methylpropyl)-2-(ethoxymethyl)-1H-imidazo[4,5-c]quinolin-4-amine, 1-{4-[(3,5-dichlorophenypsulfonyl]butyl}-2-ethyl-1H-imidazo[4,5-c]quinolin-4-amine, N-(2-{2-[4-amino-2-(ethoxymethyl)-1H-imidazo[4,5-c]quinolin-1-yl]ethoxy}ethyl)-n′-cyclohexylurea, N-{3-[4-amino-2-(ethoxymethyl)-1H-imidazo[4,5-c]quinolin-1-yl]propyl}-n′-(3-cyanophenyl)thiourea, N-[3-(4-amino-2-butyl-1H-imidazo[4,5-c]quinolin-1-yl)-2,2-dimethylpropyl]benzamide, 2-butyl-1-[3-(methylsulfonyl)propyl]-1H-imidazo[4,5-c]quinolin-4-amine, N-{2-[4-amino-2-(ethoxymethyl)-1H-imidazo[4,5-c]quinolin-1-yl]-1,1-dimethylethyl}-2-ethoxyacetamide, 1-[4-amino-2-ethoxymethyl-7-(pyridin-4-yl)-1H-imidazo[4,5-c]quinolin-1-yl]-2-methylpropan-2-ol, 1-[4-amino-2-(ethoxymethyl)-7-(pyridin-3-yl)-1H-imidazo[4,5-c]quinolin-1-yl]-2-methylpropan-2-ol, N-{3-[4-amino-1-(2-hydroxy-2-methylpropyl)-2-(methoxyethyl)-1H-imidazo[4,5-c]quinolin-7-yl]phenyl}methanesulfonamide, 1-[4-amino-7-(5-hydroxymethylpyridin-3-yl)-2-(2-methoxyethyl)-1H-imidazo[4,5-c]quinolin-1-yl]-2-methylpropan-2-ol, 3-[4-amino-2-(ethoxymethyl)-7-(pyridin-3-yl)-1H-imidazo[4,5-c]quinolin-1-yl]propane-1,2-diol, 1-[2-(4-amino-2-ethoxymethyl-1H-imidazo[4,5-c]quinolin-1-yl)-1,1-dimethylethyl]-3-propylurea, 1-[2-(4-amino-2-ethoxymethyl-1H-imidazo[4,5-c]quinolin-1-yl)-1,1-dimethylethyl]-3-cyclopentylurea, 1-[(2,2-dimethyl-1,3-dioxolan-4-yl)methyl]-2-(ethoxymethyl)-7-(4-hydroxymethylphenyl)-1H-imidazo[4,5-c]quinolin-4-amine, 4-[4-amino-2-ethoxymethyl-1-(2-hydroxy-2-methylpropyl)-1H-imidazo[4,5-c]quinolin-7-yl]-N-methoxy-N-methylbenzamide, 2-ethoxymethyl-N1-isopropyl-6,7,8,9-tetrahydro-1H-imidazo[4,5-c]quinoline-1,4-diamine, 1-[4-amino-2-ethyl-7-(pyridin-4-yl)-1H-imidazo[4,5-c]quinolin-1-yl]-2-methylpropan-2-ol, N-[4-(4-amino-2-ethyl-1H-imidazo[4,5-c]quinolin-1-yl)butyl]methanesulfonamide, and N-[4-(4-amino-2-butyl-1H-imidazo[4,5-c][1,5]naphthyridin-1-yl)butyl]-n′-cyclohexylurea. In further individual embodiments the immunotherapeutic is selected from any subset of the above list.

In some embodiments, the immunotherapeutic comprises resiquimod.

In some embodiments, the immunotherapeutic is provided in an amount that is capable of: (1) inducing IFN-α in an enriched human blood DCs; (2) inducing TNF-α in an enriched human blood DCs; and/or (3) inducing IL-12-α in an enriched human blood DCs.

In some embodiments, the immunotherapeutic is provided in an amount is capable of activating a human plasmacytoid dendritic cell, myeloid dendritic cell, or NK cell, or any combination thereof.

In some embodiments, the immunotherapeutic is administrated in combination with an a targeted therapeutic against a cancer.

In some embodiments, the targeted therapeutic is capable of binding to a tumor cell specifically or preferably in comparison to a non-tumor cell.

In some embodiments, the tumor cell is of a carcinoma, a sarcoma, a lymphoma, a myeloma, or a central nervous system cancer.

In some embodiments, the targeted therapeutic is capable of binding to a tumor antigen specifically or preferably in comparison to a non-tumor antigen.

In some embodiments, the tumor antigen is selected from the group consisting of: CD2, CD19, CD20, CD22, CD27, CD33, CD37, CD38, CD40, CD44, CD47, CD52, CD56, CD70, CD79, and CD137.

In some embodiments, the tumor antigen is selected from the group consisting of: 4-1BB, 5T4, AGS-5, AGS-16, Angiopoietin 2, B7.1, B7.2, B7DC, B7H₁, B7H₂, B7H₃, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin αν, Integrin ανβ, KIR, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2,VEGFR-1, VEGFR-2, VEGFR-3, and variants thereof. In further individual embodiments the tumor antigen is selected from any subset of the above list.

In some embodiments, the targeted therapeutic comprises an immunoglobulin, a protein, a peptide, a small molecule, a nanoparticle, or a nucleic acid.

In some embodiments, the targeted therapeutic comprises an antibody, or a functional fragment thereof.

In some embodiments, the antibody is selected from the group consisting of: Rituxan (rituximab), Herceptin (trastuzumab), Erbitux (cetuximab), Vectibix (Panitumumab), Arzerra (Ofatumumab), Benlysta (belimumab), Yervoy (ipilimumab), Perjeta (Pertuzumab), Tremelimumab, Nivolumab, Dacetuzumab, Urelumab, MPDL3280A, Lambrolizumab, Blinatumomab, CT-011, MK-3475, BMS-936559, MED14736, MSB0010718C, and margetuximab (MGAH₂₂). In further individual embodiments the antibody is selected from any subset of the above list.

In some embodiments, the targeted therapeutic comprises a Fab, Fab′, F(ab′)2, single domain antibody, T and Abs dimer, Fv, scFv, dsFv, ds-scFv, Fd, linear antibody, minibody, diabody, bispecific antibody fragment, bibody, tribody, sc-diabody, kappa (lamda) body, BiTE, DVD-Ig, SIP, SMIP, DART, or an antibody analogue comprising one or more CDRs.

In some embodiments, the targeted therapeutic comprises a ATWLPPR polypeptide of VEGFR, Thrombospondin-1 mimetics, CDCRGDCFCG (cyclic) polypeptide, SCH 221153 fragment, NCNGRC (cyclic) polypeptide, CTTHWGFTLC polypeptide, CGNKRTRGC polypeptide (LyP-1), Octreotide, Vapreotide, Lanreotide, C-3940 polypeptide, Decapeptyl, Lupron, Zoladex, or Cetrorelix.

In some embodiments, the targeted therapeutic comprises extracellular domains (ECD) or a soluble form of PD-1, PDL-1, CTLA4, BTLA, KIR, TIM3, 4-1BB, LAG3, full length of partial of a surface ligand amphiregulin, betacellulin, EGF, ephrin, epigen, epiregulin, IGF, neuregulin, TGF, TRAIL, or VEGF.

In some embodiments, the immunotherapeutic is delivered systemically.

In some embodiments, the immunotherapeutic is administered by oral administration or parenteral injection.

In some embodiments, the immunotherapeutics is administrated by intravenous injection or intratumoral injection.

In some embodiments, the abnormal cell proliferation comprises a pre-cancerous lesion.

In some embodiments, the abnormal proliferation is of cancer cells.

In some embodiments, the cancer is selected from the group consisting of: Acute myeloid leukemia (AML), Breast cancer, Chronic lymphocytic leukemia (CLL), Chronic myelogenous leukemia (CML), Hodgkin lymphoma, Multiple myeloma, Mycosis fungoides, Neuroblastoma, Non-Hodgkin lymphoma (NHL), Ovarian cancer, and Retinoblastoma.

In some embodiments, the method comprises administering to the subject an oral formulation comprising said immunotherapeutic in a dose of from about 0.0005 mg/kg, 0.0006 mg/mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, or 0.01 mg/kg, to about 0.02 mg/kg, all inclusive, twice per week.

In some embodiments, the method comprises administering to the subject an oral formulation comprising said immunotherapeutic in a dose of from 0.0001mg/kg to less than or about 0.0005mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, or 0.01 mg/kg, twice per week.

In some embodiments, the method comprises administering to the subject an intravenous formulation comprising said immunotherapeutic in a dose of from about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, or 0.006 mg/kg to about 0.015 mg/kg, all inclusive, weekly.

In some embodiments, the method comprises administering to the subject an intravenous formulation comprising said immunotherapeutic in a dose of from 0.001mg/kg to less than or about 0.003 mg/kg, 0.004 mg/kg, 0.005 mg/kg, or 0.006 mg/kg, to about 0.01 mg/kg, weekly.

In some embodiments, the immunotherapeutic in the subject has a local concentration that is from about 0.005 μg/ml to about 12 μg/ml.

In some embodiments, the immunotherapeutic in the subject has a local concentration that is from about 0.05 μg/ml, 0.1 μg/ml, 0.15 μg/ml, 0.2 μg/ml, 0.3 μg/ml, or 0.4 μg/ml, to about 0.5 μg/ml.

In some embodiments, the method comprises administering to the subject an intravenous formulation comprising the chemotherapeutics in a dose of about 40-50 mg/kg in divided dose over 2-5 days.

In some embodiments, the immunotherapeutic is administered repeatedly at intervals of 2, 3, or 4 weeks within each cycle.

In some embodiments, the method comprises administering to the subject an intravenous formulation comprising the chemotherapeutic in a dose of about 10 to 15 mg/kg, given every 7 to 10 days.

In some embodiments, the method comprises administering to the subject an intravenous formulation comprising the chemotherapeutic in a dose of about 3 to 5 mg/kg, twice weekly.

In some embodiments, the method comprises administering to the subject an intravenous formulation comprising the chemotherapeutic in a dose of about 60-120 mg/m²/day, continuous infusion.

In some embodiments, the method comprises administering to the subject an oral formulation comprising the chemotherapeutics in a dose of about 400-1000 mg/m² divided over 4-5 days.

In some embodiments, the method comprises administering to the subject an intravenous formulation comprising the chemotherapeutic in a dose of about 50-100 mg/m²/day, or 1-5 mg/kg/day.

In a further aspect, the presently disclosed embodiments provide a kit that contains the therapeutic combination provided herein, and optionally instructions for use.

### BRIEF DESCRIPTION OF THE DRAWINGS

The novel features of the invention are set forth with particularity in the appended claims. A better understanding of the features and advantages of the presently disclosed embodiments will be obtained by reference to the following detailed description that sets forth illustrative embodiments, in which the principles of the invention are utilized, and the accompanying drawings of which:

FIG. 1A-B depicts evaluation of the antitumor effects of TLRL with anti-PD-L1 and Gemcitibine combination therapy in NR-S1 syngeneic tumor model. FIG. 1A: NR-S1 cells were inoculated into C₃H mice, and treatments were started when tumor volume reached 30mm³. Control reagents (PBS or rat IgG), resiquimod (RQ) (data not shown), anti-PD-L1 mAb (PD-L1) (data not shown) and both resiquimod and the antibody (RQ+PD) were injected i.p. every three days for 4 times. Gemcitabine (30 mg/kg) was intraperitoneally administrated one day before the start of treatment with resiquimod and PD-L1 mAb (+Gem). Tumor volume was measured every day and data shown are combined results from two independent experiments (n=11). The three smaller panels on the left show the individual growth curves. The tumor growth curve and tumor volumes 14 days after the start of treatment (at day 24) are shown. FIG. 1B: Spleens were collected 24 days after the tumor inoculation, and the total cell numbers were counted (left panel). Splenocyte and red blood cell-depleted peripheral blood leukocytes were stained with anti-CD11b and anti-Gr-1 mAbs and analyzed by flow cytometry (center and right panel, respectively). The percentages of Gr-1highCD11b⁺ cells are shown. Values for splenocytes are the mean±SD (n=11) of pooled two independent experiments with similar results. Values for peripheral blood are the mean±SD from a group of six mice. Statistically different (*=p<0.05)

FIG. 2A-B depicts cell surface marker profiles of CD11b⁺ cells from NR-S1 and SCCVII tumors. The two tumor types exhibited different profiles in the TME. Ly6GhighLy6C-F4/80-cells predominated in NR-S1 tumors, and Ly6GlowLy6Clow/-F4/80+cells predominated in SCCVII tumors. NR-S1 or SCCVII tumor cells were inoculated subcutaneously and tumor masses and spleens were resected when the tumor volume reached approximately 1500 mm³ FIG. 2A: Tumor-infiltrating lymphocytes-fractions were stained and electronic gated on CD45⁺ FSClow-high large-lymphocytes (CD45⁺ gate), FSClowCD3⁺CD11b-lymphocytes (CD3⁺ T-gate), FSClow-highCD3-CD11b⁺ myeloid cells (CD11b⁺ gate), Ly6GhighLy6Clow-nega (Fr-1 gate), and Ly6GlowLy6Clow-nega (Fr-2 gate) and the percentages or expression profiles (as contour plots) of the indicated populations are shown. For T cell analysis, the proportions of CD8⁺CD4⁻(CD8⁺ T), CD8⁻CD4⁺ Foxp3⁻ (conventional T, CD4⁺ Tcon), CD8⁻CD4⁺Foxp3⁺ (Treg) are shown. FIG. 2B: Total cell numbers of spleens were counted and cells were stained with fluorochrome-conjugated anti-CD3, anti-CD11b, anti-Ly6C, and Ly6G mAbs and analyzed by flow cytometry. An electronic gate was placed on CD3⁻CD11b⁺ cells (CD11b⁺ gate), and the expression profiles of Ly6C and Ly6G are shown as contour plots. All quadrant markers were positioned to include >95-98% of control fluorochrome-stained cells. Values shown are mean±SD (n=5). *p<0.05.

FIG. 3 depicts tumor growth following inoculation of 4T1 cells into Balb/c mice, with treatments started when tumor volume reached ˜100 mm³ Control reagents (PBS), resiquimod (RQ), anti-PD-L1 mAb (PD) and both the immunotherapeutic and the antibody (RQ+PD) were injected i.v. two times, a week apart. Gemcitabine (30 mg/kg) was intraperitoneally administrated one day before the start of treatment with resiquimod and PD-L1 mAb (+Gem). Tumor volume was measured on days 14, 17 and 21 and data shown are from one independent experiment (n=6). The five smaller panels below show the individual growth curves.

### DETAILED DESCRIPTION OF THE INVENTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The presently disclosed embodiments is not limited by the illustrated ordering of acts or events, as some acts may occur in different orders and/or concurrently with other acts or events.

Furthermore, not all illustrated acts or events are required to implement a methodology in accordance with the presently disclosed embodiments.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

### I. Definitions and Abbreviations

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well-known and commonly employed in the art. Standard techniques are used for nucleic acid and peptide synthesis. The techniques and procedures are generally performed according to conventional methods in the art and various general references, which are provided throughout this document. The nomenclature used herein and the laboratory procedures in analytical chemistry, and organic synthetic described below are those well-known and commonly employed in the art. Standard techniques, or modifications thereof, are used for chemical syntheses and chemical analyses.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups, are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the presently disclosed embodiments. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH_{2,}—S(O)—CH₃, —CH₂—CH₂—S(O)₂—CH₃, —CH═CH—O—CH₃, —Si(CH₃)3, —CH₂—CH═N—OCH₃, and CH═CH—N(CH₃)—CH₃. Up to two heteroatoms may be consecutive, such as, for example, —CH₂—NH—OCH₃ and CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and CH₂—S—CH₂—CH₂—NH—CH₂—. For heteroalkylene groups, heteroatoms can also occupy either or both of the chain termini (e g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the presently disclosed embodiments.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1 (1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1 piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

As used herein, the term “haloalkyl” refers to an alkyl as defined herein, that is substituted by one or more halo groups as defined herein. Preferably the haloalkyl can be monohaloalkyl, dihaloalkyl or polyhaloalkyl including perhaloalkyl. A monohaloalkyl can have one iodo, bromo, chloro or fluoro within the alkyl group. Dihaloalkyl and polyhaloalkyl groups can have two or more of the same halo atoms or a combination of different halo groups within the alkyl. Preferably, the polyhaloalkyl contains up to 12, 10, or 8, or 6, or 4, or 3, or 2 halo groups. Non-limiting examples of haloalkyl include fluoromethyl, difluoromethyl, trifluoromethyl, chloromethyl, dichioromethyl, trichioromethyl, pentafluoroethyl, heptafluoropropyl, difluorochioromethyl, dichiorofluoromethyl, difluoroethyl, difluoropropyl, dichloroethyl and dichioropropyl. A perhaloalkyl refers to an alkyl having all hydrogen atoms replaced with halo atoms.

As used herein, the term “heteroaryl” refers to a 5-14 membered monocyclic- or bicyclic- or fused polycyclic-ring system, having 1 to 8 heteroatoms selected from N, O, S or Se. Preferably, the heteroaryl is a 5-10 membered ring system. Typical heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2, 3-triazolyl, tetrazolyl, 2-, 3-, or 4-pyridyl, 3- or 4-pyridazinyl, 3-, 4-, or 5-pyrazinyl, 2-pyrazinyl, 2-, 4-, or 5-pyrimidinyl.

The term “heteroaryl” also refers to a group in which a heteroaromatic ring is fused to one or more aryl, cycloaliphatic, or heterocycloalkyl rings, where the radical or point of attachment is on the heteroaromatic ring. Nonlimiting examples include but are not limited to 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinoliyl, 1-, 3-, 4-, 5-, 6-, 7-, or 8-isoquinoliyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4aH carbazolyl, 1-, 2-, 3-, 4-, 5-, 6-, 7-, or 8-carbzaolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1- , 2-, 3-, 4-, 5-, 6-, 7-, 8-, or 9-acridinyl, 1-, 2-, 4-, 5-, 6-, 7-, 8-, or 9-perimidinyl, 2-, 3-, 4-, 5-, 6-, 8-, 9-, or 10- phenathrolinyl, 1-, 2- , 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenoxazinyl, 2-, 3-, 4-, 5-, 6-, or 1-, 3-, 4-, 5-, 6-, 7-, 8-, 9-, or 10-benzisoqinolinyl, 2-, 3-, 4-, or 5-thieno[2,3-b]furanyl, 2-, 3-, 5-, 6-, 7-, 8-, 9-, 10 -, or 11-7H-pyrazino[2,3-c]carbazolyl,2-, 3-, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 54H-imidazo[4,5-d]thiazolyl, 3-, 5-, or 8-pyrazino[2,3-d]pyridazinyl, 2-, 3-, 5-, or 6-imidazo[2,1-b] thiazolyl, 1-, 3-, 6-, 7-, 8-, or 9-furo[3,4-c]cinnolinyl, 1-, 2-, 3-, 4-, 5-, 6-, 8-, 9-, 10, or 11-4H-pyrido[2,3-c]carbazolyl, 2-, 3-, 6-, or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 11-1H-pyrrolo[1,2-b][2]benzazapinyl. Typical fused heteroaryl groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5- , 6-, or 7-benzoxazolyl, 2-, 4-, 5-, 6-, or 7-benzimidazolyl, 2-, 4-, 5-, 6-, or 7-benzothiazolyl.

As used herein, the term “heterocyclyl” or “heterocyclo” refers to an optionally substituted, fully saturated or unsaturated, aromatic or nonaromatic cyclic group, e.g., which is a 4- to 7-membered monocyclic, 7- to 12-membered bicyclic or 10- to 15-membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized. The heterocyclic group may be attached at a heteroatom or a carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, triazolyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, 1,1,4-trioxo-1,2,5-thiadiazolidin-2-yl and the like.

Exemplary bicyclic heterocyclic groups include indolyl, dihydroidolyl, benzothiazolyl, benzoxazinyl, benzoxazolyl, benzothienyl, benzothiazinyl, quinuclidinyl, quinolinyl, tetrahydroquinolinyl, decahydroquinolinyl, isoquinolinyl, tetrahydroisoquinolinyl, decahydroisoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]-pyridinyl]or furo[2,3-b]pyridinyl), dihydroisoindolyl, 1,3-dioxo-1,3-dihydroisoindol-2-yl, dihydroquinazolinyl (such as 3,4-dihydro-4-oxo-quinazolinyl), phthalazinyl and the like.

Exemplary tricyclic heterocyclic groups include carbazolyl, dibenzoazepinyl, dithienoazepinyl, benzindolyl, phenanthrolinyl, acridinyl, phenanthridinyl, phenoxazinyl, phenothiazinyl, xanthenyl, carbolinyl and the like.

The term “heterocyclyl” further refers to heterocyclic groups as defined herein substituted with 1, 2 or 3 substituents selected from the groups consisting of the following:
(a) alkyl;
(b) hydroxy (or protected hydroxy);
(c) halo;
(d) oxo, i.e., ═O;
(e) amino, alkylamino or dialkylamino;
(f) alkoxy;
(g) cycloalkyl;
(h) carboxy;
(i) heterocyclooxy, wherein heterocyclooxy denotes a heterocyclic group bonded through an oxygen bridge;
(j) alkyl-O—C(O)—;
(k) mercapto;
(l) nitro;
(m) cyano;
(n) sulfamoyl or sulfonamido;
(o) aryl;
(p) alkyl-C(O)—O—;
(q) aryl-C(O)—O—;
(r) aryl-S—;
(s) aryloxy;
(t) alkyl-S—;
(u) formyl, i.e., HC(O)—;
(v) carbamoyl;
(w) aryl-alkyl-; and
(x) aryl substituted with alkyl, cycloalkyl, alkoxy, hydroxy, amino, alkyl-C(O)—NH—, alkylamino, dialkylamino or halogen.

As used herein, the term “alkenyl” refers to a straight or branched hydrocarbon group having 2 to 20 carbon atoms and that contains at least one double bonds. The alkenyl groups preferably have about 2 to 8 carbon atoms.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroakyl substituents,” respectively, and they can be one or more of a variety of groups selected from, but not limited to: —OR′, ═), ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R′)═NR′″, —S(O)R′, —S(O)₂R′, —S(O)₂NR′R″, —NRSO₂R′, —CN and NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R¹ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R″ and R″″ groups when more than one of these groups is present.

Two of the aryl substituents on adjacent atoms of the aryl or heteroaryl ring may optionally be replaced with a substituent of the formula T-C(O)—(CRR′)_{q}—U—, wherein T and U are independently NR—, —O—, —CRR′— or a single bond, and q is an integer of from 0 to 3. Alternatively, two of the substituents on adjacent atoms of the aryl or heteroaryl ring may optionally be replaced with a substituent of the formula A-(CH₂)₁-B-, wherein A and B are independently CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula (CRR′)ₛ—X—(CR″R)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆) alkyl.

As used herein, the term “heteroatom” includes oxygen (O), nitrogen (N), sulfur (S), phosphorus (P) and silicon (Si).

As used herein, the term “aryloxy” refers to both an -O-aryl and an -O-heteroaryl group, wherein aryl and heteroaryl are defined herein.

As used herein, the term “pharmaceutically acceptable salts” refers to salts that retain the biological effectiveness and properties of the compounds of this invention and, which are not biologically or otherwise undesirable. In many cases, the compounds of the presently disclosed embodiments are capable of forming acid and/or base salts by virtue of the presence of amino and/or carboxyl groups or groups similar thereto (e.g., phenol or hydroxyamic acid). Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of the presently disclosed embodiments can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in Remington's Pharmaceutical Sciences, 20th ed., Mack Publishing Company, Easton, Pa., (1985), which is herein incorporated by reference.

As used herein, the term “pharmaceutically acceptable carrier/excipient” includes any and all solvents, dispersion media, coatings, surfactants, antioxidants, preservatives (e.g., antibacterial agents, antifungal agents), isotonic agents, absorption delaying agents, salts, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except in so far as any conventional carrier is incompatible with the active ingredient, its use in the therapeutic or pharmaceutical compositions is contemplated.

As used herein, the term “subject” refers to an animal Preferably, the animal is a mammal A subject also refers to for example, primates (e.g., humans), cows, sheep, goats, horses, dogs, cats, rabbits, rats, mice, fish, birds and the like. In a preferred embodiment, the subject is a human

As used herein, the term “therapeutic combination” or “combination” refers to a combination of one or more active drug substances, i.e., compounds having a therapeutic utility, particularly a combination of a chemotherapeutic and an immunotherapeutic, or a combination of a chemotherapeutic, an immunotherapeutic, and a targeted therapeutic, as herein described. Typically, each such compound in the therapeutic combinations of the presently disclosed embodiments will be present in a pharmaceutical composition comprising that compound and a pharmaceutically acceptable carrier. The compounds in a therapeutic combination of the presently disclosed embodiments may be administered simultaneously or separately, as part of a regimen. In particular embodiments various components of a combination can be formulated separately or together, administered by the same or different routes of administration, or administered according to the same or different schedules. However, even when administered by different routes or on different schedules, administration is coordinated so that the subject receives a greater benefit, in terms of greater therapeutic effect and/or reduced adverse side-effect, than if the components were not all administered or were administered without the coordination.

### II. Compositions

In general, the presently disclosed embodiments provide therapeutic combinations, pharmaceutical compositions, and methods for treating cancers using combination therapy; more specifically, the combination of a chemotherapeutic that can reduce myeloid-derived suppressor cells (MDSC) and immunotherapy (such as using Toll-like Receptor Ligand “TLRL”), for example, a TLR7 and 8 dual agonist, to activate DCs in innate immunity. In further embodiments the combination will also comprise a targeted therapeutic.

In one aspect, the presently disclosed embodiments provide a method for treating tumor or abnormal cell proliferation, in a subject that is in need of such treatment, comprising administering to the subject: a chemotherapeutics that is provided in an amount that is capable of reducing MDSCs population in blood, spleen, and/or tumor microenvironment in said subject; and an effective amount of an immunotherapeutic. In some embodiments the effective amount of the immunotherapeutic is less when used as part of the combination than if the immunotherapeutic was used alone, or if is used was not coordinated with the use of the chemotherapeutic.

A therapeutic combination may be provided using more than one pharmaceutical composition. In such embodiments, a chemotherapeutic may be provided in one pharmaceutical composition and an immunotherapeutic may be provided in a second pharmaceutical composition so that the two compounds can be administered separately such as, for example, at different times, by different routes of administration, and the like. Thus, it also may be possible to provide the chemotherapeutic and the immunotherapeutic in different dosing regimens. Similarly, when a targeted therapy is additionally included in the combination it may be provided in a third pharmaceutical composition and administered independently of one or both of the other components of the combination, though still a coordinated manner.

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

In general, the chemotherapeutic and the immunotherapeutic are not linked to each other, such as by a covalent linker. In some embodiments the chemotherapeutic and/or the immunotherapeutic is/are not co-formulated with a tumor cell or with another tumor-associated antigen-containing immunogenic composition. In still further embodiments administration of the chemotherapeutic and/or the immunotherapeutic is/are not coordinated with administration of a tumor cell or with another tumor-associated antigen-containing immunogenic composition.

### A. Chemotherapeutics

In general, the method provided herein comprise administering to a subject first a chemotherapeutic that is provided in an amount that is capable of reducing MDSC population in blood, spleen, and/or tumor microenvironment in the subject, prior to administrating an immunotherapeutic, and optionally, a targeted therapeutic.

In some embodiments, the chemotherapeutic comprises a myeloid-derived suppressor cells (MDSC) inhibitor.

By “myeloid-derived suppressor cells (MDSC) inhibitor” herein is meant a therapeutic agent that is able to regulate immune suppressive cells such as MDSC. In general, the MDSC inhibitor causes the induction of MDSC apoptosis and/or necrosis, or cytotoxicity by inhibition of c-kit, or VEGFR, or ARG1, or iNOS, or 5100 or MMPs functions of MDSCs and ROS ERK activation or antioxidant genes. In some embodiments, the MDSC inhibitor is selected from the group consisting of: Paclitaxel, Gemcitabine, 5-Fluorouracile, Oxaliplatin, Cisplatin, Carboplatin, Dasatinib, Sunitinib, and Doxorubicin.

### B. Immunotherapeutics

In general, the combinations or compositions of the presently disclosed embodiments comprise an immunotherapeutic.

By “immunotherapeutic” herein is meant a compound, a molecule, or an agent that is capable of stimulating or enhancing the body's immune system Immunotherapeutics are used for the treatment of disease by inducing, enhancing, or suppressing an immune response. Immunotherapeutics of the presently disclosed embodiments generally are designed to elicit or amplify an immune response, rather than suppress an immune response. However, in some embodiments the primary effect of the immunotherapeutic can be to suppress differentiation or proliferation of immunosuppressive cells rather than to more directly elicit or amplify an anti-tumor immune response.

In general, the immunotherapeutics of the presently disclosed embodiments act, directly or indirectly, on toll like receptors, nucleotide-oligomerization domain-like receptors, RIG-I-Like receptors, c-type lectin receptors, or cytosolic DNA sensors, or a combination thereof. Particularly, the immunotherapeutics of the presently disclosed embodiments are capable of activating human plasmacytoid dendritic cells, myeloid dendritic cells, NK cells, or tumor cell, or a combination thereof.

In some embodiments, the immunotherapeutics of the presently disclosed embodiments activate human immune cells, including but not limited to dendritic cells, macrophages, monocytes, myeloid-derived suppressor cells, NK cells, B cells, T cells, or tumor cells, or a combination thereof.

Dendritic cells are the most potent antigen-presenting cells. Dendritic cells play an essential role for the initiation of both innate and adaptive immune responses. Dendritic cells also play a key role in the induction and maintenance of immune tolerance.

By “dendritic cells” (DC) herein is meant a heterogeneous cell population including two main subtypes: namely, myeloid DC (mDC) and plasmacytoid DC (pDC) (Steinman et al., 1979, J. Exp. Med., 149, 1-16). These two blood DC subsets were originally differentiated by their expression of CD11c (integrin complement receptor) and CD123 (IL-3Rα). Each of the pDC and mDC populations constitutes between about 0.2 to about 0.6% of the PBMC population in humans.

By “pDC” herein is meant plasmacytoid dendritic cells and they represent a subtype of dendritic cells found in the blood and peripheral lymphoid organs. These cells express the surface markers CD123, BDCA-2(CD303) and BDCA-4(CD304) and HLA-DR, but do not express CD11c, CD14, CD3, CD20 or CD56, which distinguishes them from conventional dendritic cells, monocytes, T-cells, B cells and NK cells. As components of the innate immune system, these cells express intracellular Toll-like receptors 7 and 9, which enable the detection of viral and bacterial nucleic acids, such as ssRNA or CpG DNA motifs. Upon stimulation and subsequent activation, these cells produce large amounts of Type I interferon (mainly IFN-α and IFN-β) and Type III interferon (e.g., IFN-λ) which are critical pleiotropic anti-viral compounds mediating a wide range of effects. By generating a large amount of type I interferon, cytokines and chemokines, plasmacytoid dendritic cells are widely involved in the body's innate and adaptive immune responses. They can regulate NK cells, T cells, B cells and other cells involved in immune response intensity, duration, and response mode, thus play a very important function in tumor, infection and autoimmune disease. (Liu Y J. IPC: professional type 1 interferon-producing cells and plasmacytoid dendritic cell precursors. Annual Rev Immunol. 2005; 23:275-306. Gilliet M, Cao W, Liu Y J. Plasmacytoid dendritic cells: sensing nucleic acids in viral infection and autoimmune diseases. Nat Rev Immunol. 2008 August; 8 (8) :594-606).

By “mDC” herein is meant myeloid dendritic cells and they represent a subtype of circulating dendritic cells found in blood and peripheral lymphoid organs. These cells express the surface markers CD11c, CD1a, HLA-DR and either BDCA-1 (CD1c) or BDCA-3 (CD141). They do not express BDCA-2 or CD123, which distinguishes them from pDC. mDC also do not express CD3, CD20 or CD56. As components of the innate immune system, mDC express Toll-like receptors (TLR), including TLR2, 3, 4, 5, 6 and 8, which enable the detection of bacterial and viral components. Upon stimulation and subsequent activation, these cells are the most potent antigen presenting cells to activate antigen-specific CD4 as well as CD8 T cells. In addition, mDCs has the ability to produce large amounts of IL-12 and IL23, which is critical for the induction of Th1-mediated or Th17 cell-mediated immunity.

Studies have found that many solid tumors, such as breast cancer, head and neck cancer, and ovarian cancer have resident pDCs (Treilleux I, Blay J Y, Bendriss-Vermare N et al. Dendritic cell infiltration and prognosis of early stage breast cancer, Clin Cancer Res 2004, 10:7466-7474; Hartmann E, Wollenberg B, Rothenfusser S et al., Identification and functional analysis of tumor-infiltrating plasmacytoid dendritic cells in head and neck cancer, Cancer Res 2003; 63:6478-6487; Zou W P, Machelon V, Coulomb-L'Hermin A, et al. Stromal-derived factor-1 in human tumors recruits and alters the function of plasmacytoid precursor dendritic cells, Nat Med 2001, 7:1339-1346) and factors secreted by tumor cells inhibit DC maturation. (Gabrilovich D I, Corak J, Ciernik I F et al., Decreased antigen presentation by dendritic cells in patients with breast cancer, Clin Cancer Res 1997, 3:483-490; Bell D, Chomarat P, Broyles D et al., In breast carcinoma tissue, immature dendritic cells reside within the tumor, whereas mature dendritic cells are located in peritumoral areas, J Exp Med 1999, 190:1417-1425; Menetrier-Caux C, Montmain G, Dieu M C et al. Inhibition of the differentiation of dendritic cells from CD34 (+) progenitors by tumor cells: role of interleukin-6 and macrophage colony-stimulating factor, Blood 1998, 92:4778-4791). These immature DC did not play a role in promoting anti-tumor immunity. By contrast, DCs within the tumor microenvironment promote tumor growth by inhibiting antitumor immunity and by promoting angiogenesis. There is evidence that Toll-like receptor 7 agonist Imiquimod, and Toll-like receptor 9 agonist CpG drugs can stimulate pDC within the tumor microenvironment to inhibit tumor development (Dummer R, Urosevic M, Kempf W et al., Imiquimod in basal cell carcinoma: how does it work?, Br J Dermatol 2003, 149:57-58; Miller R L, Gerster J F, Owens M L et al., Imiquimod applied topically: a novel immune response modifier and new class of drug, Int J Immunopharmacol 1999, 21:1-14; Hofmann M A, Kors C, Audring H et al., Phase 1 evaluation of intralesionally injected TLR9-agonist PF-3512676 in patients with basal cell carcinoma or metastatic melanoma, J Immunother 2008, 31:520-527).

Natural killer (NK) cells are a type of cytotoxic lymphocyte that constitutes a major component of the immune system. NK cells are a subset of peripheral blood lymphocytes defined by the expression of CD56 or CD 16 and the absence of the T cell receptor (CD3). They recognize and kill transformed cell lines without priming in an MHC-unrestricted fashion. NK cells play a major role in the rejection of tumors and cells infected by viruses. The process by which an NK cell recognizes a target cell and delivers a sufficient signal to trigger target lysis is determined by an array of inhibitory and activating receptors on the cell surface. NK discrimination of self from altered self involves inhibitory receptor recognition of MHC-I molecules and non-MHC ligands like CD48 and Clr-1b. NK recognition of infected or damaged cells (altered self) is coordinated through stress induced ligands (e.g., MICA, MICE, Rael, H₆₀, Mult1) or virally encoded ligands (e.g., m157, hemagluttinin) recognized by various activating receptors, including NKG2D, Ly49H and NKp46/Ncr 1.

NK cells represent the predominant lymphoid cell in the peripheral blood for many months after allogeneic or autologous stem cell transplant and they have a primary role in immunity to pathogens during this period (Reittie et al., (1989) Blood 73: 1351-1358; Lowdell et al., (1998) Bone Marrow Transplant 21: 679-686). The role of NK cells in engraftment, graft-versus-host disease, anti-leukemia activity and post-transplant infection is reviewed in Lowdell (2003) Transfusion Medicine 13:399-404.

Human NK cells mediate the lysis of tumor cells and virus-infected cells via natural cytotoxicity and antibody-dependent cellular cytotoxicity (ADCC).

Human NK cells are controlled by positive and negative cytolytic signals. Negative (inhibitory) signals are transduced by C-lectin domain containing receptors CD94/NKG2A and by some Killer Immunoglobulin-like Receptors (KIRs). The regulation of NK lysis by inhibitory signals is known as the “missing self” hypothesis in which specific HLA-class I alleles expressed on the target cell surface ligate inhibitory receptors on NK cells. The down-regulation of HLA molecules on tumor cells and some virally infected cells (e.g. CMV) lowers this inhibition below a target threshold and the target cells may become susceptible to NK cell-mediated lysis if the target cells also carry NK-priming and activating molecules. TLR7, TLR8, or TLR9 agonists can activate both mDC and pDCs to produce type I IFNs and express costimulatory molecules such as GITR-ligand, which subsequently activate NK cells to produce IFN-γ and potently promote NK cell killing function.

Inhibitory receptors fall into two groups, those of the Ig-superfamily called Killer Immunoglobulin-like Receptors (KIRs) and those of the lectin family, NKG2, which form dimers with CD94 at the cell surface. KIRs have a 2- or 3-domain extracellular structure and bind to HLA-A, -B or -C. HLA-E is the ligand for NKG2/CD94 complexes.

Inhibitory KIRs have up to 4 intracellular domains which contain ITIMs and the best characterized are KIR2DL1, KIR2DL2 and KIR2DL3 which are known to bind HLA-C molecules. KIR2DL2 and KIR2DL3 bind the group 1 HLA-C alleles while KIR2DL1 binds to group 2 alleles. Certain leukemia/lymphoma cells express both group 1 and 2 HLA-C alleles and are known to be resistant to NK-mediated cell lysis.

With regards to positive activating signals, ADCC is thought to be mediated via CD16, and a number of triggering receptors responsible for natural cytotoxicity have been identified, including CD2, CD38, CD69, NKRP-I, CD40, B7-2, NK-TR, NKp46, NKp30 and NKp44. Several KIR molecules with short intracytoplasmic tails are also stimulatory. These KIRs (KIR2DS1, KIR2DS2 and KIR2DS4) are known to bind to HLA-C; their extracellular domains being identical to their related inhibitory KIRs. The activatory KIRs lack the ITIMs and instead associate with DAP 12 leading to NK cell activation. The mechanism of control of expression of inhibitory versus activatory KIRs remains unknown.

Several reports have described the expression of TLRs in mouse or human cancer or cancer cell lines. For example, TLR1 to TLR6 are expressed by colon, lung, prostate, and melanoma mouse tumor cell lines (Huang B, et al., Toll-like receptors on tumor cells facilitate evasion of immune surveillance, Cancer Res. 2005, 65(12):5009-5014); TLR3 is expressed in human breast cancer cells (Salaun B, Coste I, Rissoan M C, Lebecque S J, Renno T, TLR3 can directly trigger apoptosis in human cancer cells, J Immunol. 2006; 176(8):4894-4901); hepatocarcinoma and gastric carcinoma cells express TLR2 and TLR4 (Huang B, et al., Listeria monocytogenes promotes tumor growth via tumor cell toll-like receptor 2 signaling, Cancer Res. 2007, 67(9):4346-4352); TLR9 (Droemann D, et al., Human lung cancer cells express functionally active Toll-like receptor 9, Respir Res. 2005, 6:1); and TLR4 (He W, Liu Q, Wang L, Chen W, Li N, Cao X, TLR4 signaling promotes immune escape of human lung cancer cells by inducing immunosuppressive cytokines and apoptosis resistance, Mol Immunol. 2007, 44(11):2850-2859) are expressed by human lung cancer cells. TLR7 and TLR8 are found in tumor cells of human lung cancer (Cherfils-Vicini J, Platonova S, Gillard M, Laurans L, Validire P, Caliandro R, Magdeleinat P, Mami-Chouaib F, Dieu-Nosjean M C, Fridman W H, Damotte D, Sautes-Fridman C, Cremer I, J. Clin Invest. 2010, 120(4):1285-1297).

TLR are a family of proteins that sense a microbial product and/or initiates an adaptive immune response. TLRs activate a dendritic cell (DC). TLRs are conserved membrane spanning molecules containing an ectodomain of leucine-rich repeats, a transmembrane domain and an intracellular TIR (Toll/interleukin receptor) domain. TLRs recognize distinct structures in microbes, often referred to as “PAMPs” (pathogen associated molecular patterns). Ligand binding to TLRs invokes a cascade of intra-cellular signaling pathways that induce the production of factors involved in inflammation and immunity.

In some embodiments, the immunotherapeutic is a TLR7 and/or TLR8 agonist. TLR7 and TLR8 are phylogenetically and structurally related. TLR7 is selectively expressed by human pDCs and B cells. TLR8 is predominantly expressed mDCs, monocytes, macrophages and myeloid suppressor cells. TLR7-specific agonists activate plasmacytoid DCs (pDCs) to produce large amounts of type 1 IFNs and to express high levels of costimulatory molecules that promote activation of T cells, NK cells, B cells and mDCs. TLR8-specific agonists activate myeloid DCs, monocytes, macrophages or myeloid-derived suppressor cells to produce large amounts of type 1 IFN, IL-12 and IL-23, and express high levels of MHC class I, MHC class II and costimulatory molecules that promote the activation of antigen specific CD4⁺ and CD8⁺ T cells.

Dual agonists of TLR7 and TLR8 include resiquimod, CL075 (3M002), CL097, natural ssRNAs, polyU, poly(dT), GU-rich oligonucleotides such as ssRNA40, ssRNA-DR, and ORN06.

TLR7-specific agonists include CL264, CL307, gardiquimod, imiquimod, and loxoribine. TLR8-specific agonists include TL8-506 and ORN02.

In some embodiments, the immunotherapeutic is a TLR7 and/or TLR8 agonist that is represented by the structure of Formula (I):

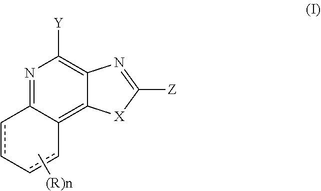

wherein dashed line represents bond or absence of bond;
X is S or —NR₁, R₁ is —WO—W₁—W₂—W₃—W₄,
WOis a bond, alkyl alkenyl, alkynyl, alkoxy, or alkyl-S-alkyl-,
W₁ is a bond, —O—, or NR₂—, wherein R₂ is hydrogen, alkyl or alkenyl,
W₂ is a bond, —O—, —C(O)—, —C(S)—, or —S(O)₂—,
W₃ is a bond, —NR₃—, wherein R₃ is hydrogen, alkyl or alkenyl,
W₄ is hydrogen, alkyl, alkenyl, alkynyl, alkoxy, cycloalkyl, aryl, aryloxy, heteroaryl, or heterocyclyl, each of which is optionally substituted by one or more substituents selected from the group consisting of hydroxyl, alkoxy, alkyl, alkenyl, alkynyl, cycloalkyl, aryl, heteroaryl, heterocyclyl, —NH₂, nitro, -alkyl-hydroxyl, -alkyl-aryl, -alkyl-heteroaryl, -alkyl-heterocyclyl, —O—R₄, —O-alkyl-R₄, -alkyl-O—R₄, —C(O)—R₄, -alkyl-C(O)—R₄, -alkyl-C(O)—O—R₄, —C(O)—O—R₄, —S—R ₄, —S(O)₂—R₄, —NH—S(O)₂—R₄, -alkyl-S—R ₄, -alkyl-S(O)₂—R₄, —NHR₄, —NR₄R₄,—NH-alkyl-R₄, halogen, —CN, —NO₂, and —SH, wherein R₄ is independently hydrogen, alkyl, alkenyl, -alkyl-hydroxyl, aryl, heteroaryl, heterocyclyl, or haloalkyl;
Z is hydrogen, alkyl, alkenyl, alkynyl, alkoxy, aryl, haloalkyl, heteroaryl, heterocyclyl, each of which can be optionally substituted by one or more substituents selected from the group consisting of hydroxyl, alkoxy, alkyl, alkenyl, alkynyl, aryl, heteroaryl, heterocyclyl, halogen, cyano, nitro, —N(R₅)₂, -alkoxy-alkyl, -alkoxy-alkenyl, —C(O)-alkyl, —C(O)—O-alkyl, —O—C(O)-alkyl, —C(O)—N(R₅)2, aryl, heteroaryl, —CO-aryl, and CO-heteroaryl, wherein each R₅ is independently hydrogen, alkyl, haloalkyl, -alkyl-aryl, or alkyl-heteroaryl;
R is hydrogen, alkyl, alkoxy, haloalkyl, halogen, aryl, heteroaryl, heterocyclyl, each of which is optionally substituted by one or more substituents selected from the group consisting of hydroxyl, alkoxy, alkyl, alkenyl, alkynyl, cycloalkyl, aryl, heteroaryl, heterocyclyl, —NH₂, nitro, -alkyl-hydroxyl, -alkyl-aryl, -alkyl-heteroaryl, -alkyl-heterocyclyl, —O—R₄, —O-alkyl-R₄, -alkyl-O—R₄, —C(O)—R₄, —C(O)—NH—R₄, —C(O)—NR₄R₄, -alkyl-C(O)—R₄, -alkyl-C(O)—O—R₄, —C(O)—O—R₄, —O—C(O)—R₄, —S—R ₄, —S(O)₂—R₄, —NH—S(O)₂—R₄, -alkyl-S—R ₄, -alkyl-S(O)₂—R₄, —NHR₄, —NR₄R₄, —NH-alkyl-R₄, halogen, —CN, and —SH, wherein R₄ is independently hydrogen, alkyl, alkenyl, alkoxy, -alkyl-hydroxyl, aryl, heteroaryl, heterocyclyl, or haloalkyl;
n is 0, 1, 2, 3, or 4;
Y is —NR₆R₂, —CR₆R₇R₈, or -alkyl-NH₂, each of which can be optionally substituted by one or more substituents selected from the group consisting of hydroxyl, alkoxy, alkyl, alkenyl, alkynyl, —NH₂, halogen, —N(R₅)₂, -alkoxy-alkyl, -alkoxy-alkenyl, —C(O)-alkyl, —C(O)—O-alkyl, —C(O)—N(R₅)2, aryl, heteroaryl, —CO-aryl, and —CO-heteroaryl,
wherein R₆, R₇ and R₈ are independently hydrogen, alkyl, alkenyl, alkoxy, alkylamino, dialkylamino, alkylthio, arylthio, -alkyl-hydroxyl, -alkyl-C(O)—O—R₉, -alkyl-C(O)—R₉, or -alkyl-O—C(O)—R₉, wherein each R₅ is independently hydrogen, alkyl, haloalkyl, -alkyl-aryl, or -alkyl-heteroaryl, wherein R₉ is hydrogen, alkyl, alkenyl, halogen, or haloalkyl;
X and Z taken together may optionally form a (5-9)-membered ring;
or a pharmaceutically acceptable salt or solvate thereof.

In some embodiments, X of Formula (I) is S.

In some embodiments, X of Formula (I) is —NR₁, R₁ is alkyl, -alkyl-W₄, -alkyl-O—W₄, -alkyl-NH—C(O)—W₄, -alkoxy-NH—C(O)—W₄, -alkyl-NH—C(O)—NH—W₄, -alkoxy-NH—C(O)—NH—W₄, -alkyl-S(O)₂-W₄, or -alkyl-NH—C(S)—W₄, wherein W₄ is defined above.

In some embodiments, Z of Formula (I) is hydrogen, alkyl, alkoxy, aryl, heteroaryl, haloalkyl, each of which is optionally substituted by one to three substituents selected from the group consisting of hydroxyl, alkyl, aryl, heteroaryl, heterocyclyl, cyano, -alkoxy-alkyl, nitro, and —N(R₅)2, wherein each R₅ is independently hydrogen, alkyl, haloalkyl, -alkyl-aryl, or -alkyl-heteroaryl.

In some embodiments, Y of Formula (I) is —NH₂, -alkyl-NH₂, each of which is optionally substituted by one to three substituents selected from the group consisting of alkyl, alkoxy, alkenyl, and alkynyl.

In some embodiments, n of Formula (I) is 1 or 2.

In some embodiments, R of Formula (I) is aryl or heteroaryl each of which is optionally substituted by one to three substituents selected from the group consisting of hydroxyl, alkoxy, -alkyl-hydroxyl, —O—R₄, —O-alkyl-R₄, -alkyl-O—R₄, —C(O)—R₄, —C(O)—NH—R₄, —C(O)—NR₄R₄, -alkyl-C(O)—R₄, -alkyl-C(O)—O—R₄, —C(O)—O—R₄, —O—C(O)—R₄, —S—R ₄, —C(O)—S—R ₄, —S—C(O)—R₄, —S(O)₂—R₄, —NH—S(O)₂—R₄, -alkyl-S—R ₄, -alkyl-S(O)₂—R₄, —NHR₄, —NR₄R_{4,}—NH-alkyl-R₄, halogen, —CN, and —SH, wherein R₄ is independently hydrogen, alkyl, alkenyl, alkoxy, -alkyl-hydroxyl, aryl, heteroaryl, heterocyclyl, or haloalkyl.

In some embodiments, the immunotherapeutic is a TLR7 and/or TLR8 agonist that is selected from Table 2. The compounds in Table 2 are described and characterized in more details in U.S. Pat. Nos. 4,689,338, 5,389,640, 5,226,575, 6,110,929, 6,194,425, 5,352,784, 6,331,539, 5,482,936, 6,451810, WO2002/46192, WO2002/46193, WO2002/46194, 2004/0014779 and US2004/0162309.

TABLE 2
Representative TLR7 and/or TLR8 Agonists

| Name | Structure |
|---|---|
| 2-propylthiazolo[4,5- c]quinolin-4-amine (CL075) | 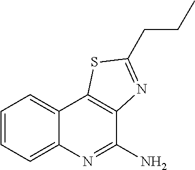 [SMILES: CCCc1nc2c(N)nc3ccccc3c2s1] |
| 1-(2-methylpropyl)-1H- imidazo[4,5-c]quinolin-4- amine (Imiquimod) | 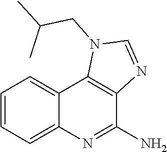 [SMILES: CC(C)Cn1cnc2c(N)nc3ccccc3c21] |
| 4-amino-2-(ethoxymethyl)- a,a-di-methyl-1H- imidazo[4,5-c]quinoline-1- ethanol (Resiquimod) | 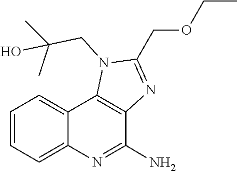 [SMILES: CCOCc1nc2c(N)nc3ccccc3c2n1CC(C)(C)O] |
| 1-(4-amino-2- ethylaminomethylimidazo- [4,5-c]quinolin-1-yl)-2- methylpropan-2-ol (Gardiquimod) | 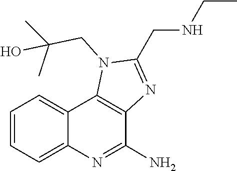 [SMILES: CCNCc1nc2c(N)nc3ccccc3c2n1CC(C)(C)O] |
| N-[4-(4-amino-2-ethyl-1H- imidazo[4,5-c]quinolin-1- yl)butyl-] methanesulfonamide (CM001) | 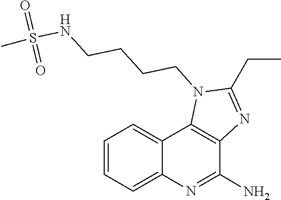 [SMILES: CCc1nc2c(N)nc3ccccc3c2n1CCCCNS(C)(=O)=O] |
| 7-allyl-7,8-dihydro-8-oxo- guanosine (Loxoribine) | 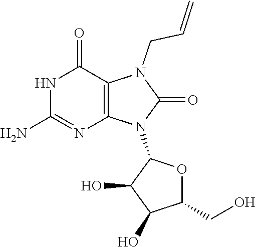 [SMILES: C=CCn1c(=O)n([C@@H]2O[C@H](CO)[C@@H](O)[C@H]2O)c2nc(N)[nH]c(=O)c21] |
| 4-amino-2-ethoxymethyl- aa-dimethyl-6,7,8,9- tetrahydro-1h-imidazo[4,5- c]quinoline-1-ethanol ol | 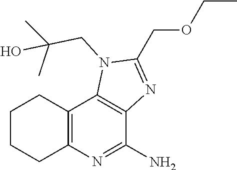 [SMILES: CCOCc1nc2c(N)nc3c(c2n1CC(C)(C)O)CCCC3] |
| 4-amino-aa-dimethyl-2- methoxyethyl-1h- imidazo[4,5-c]quinoline-1- ethanol | 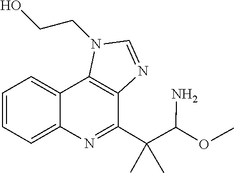 [SMILES: COC(N)C(C)(C)c1nc2ccccc2c2c1ncn2CCO] |
| 1-(2-(3- (benzyloxy)propoxy)ethyl)- 2-(ethoxymethyl)-1H- imidazo[4,5-c]quinolin-4- amine | 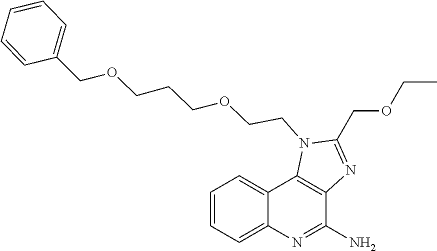 [SMILES: CCOCc1nc2c(N)nc3ccccc3c2n1CCOCCCOCc1ccccc1] |
| N-[4-(4-amino-2-butyl-1H- imidazo[4,5- c][1,5]naphthyridin-1- yl)butyl]-n′-butylurea | 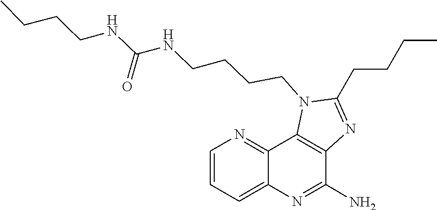 [SMILES: CCCCNC(=O)NCCCCn1c(CCCC)nc2c(N)nc3cccnc3c21] |
| N1-[2-(4-amino-2-butyl- 1H-imidazo[4,5-c][1,5] naphthyridin-1-yl)ethyl]-2- amino-4- methylpentanamide | 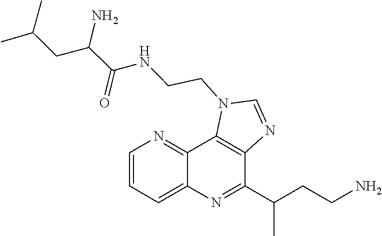 [SMILES: CC(C)CC(N)C(=O)NCCn1cnc2c(C(C)CCN)nc3cccnc3c21] |
| N-(2-{2-[4-amino-2-(2- methoxyethyl)-1H- imidazo[4,5-c]quinolin-1- yl]ethoxy}ethyl)-n′- phenylurea | 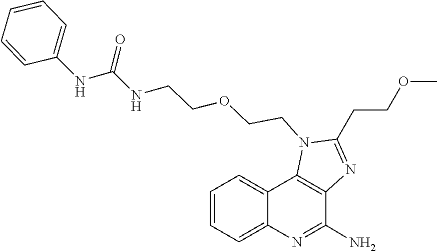 [SMILES: COCCc1nc2c(N)nc3ccccc3c2n1CCOCCNC(=O)Nc1ccccc1] |
| 1-(2-amino-2- methylpropyl)-2- (ethoxymethyl)-1H- imidazo[4,5-c]quinolin-4- amine | 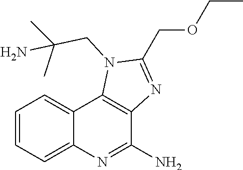 [SMILES: CCOCc1nc2c(N)nc3ccccc3c2n1CC(C)(C)N] |
| 1-{4-[(3,5- dichlorophenyl)sulfonyl] butyl}-2-ethyl- 1H-imidazo[4,5- c]quinolin-4-amine | 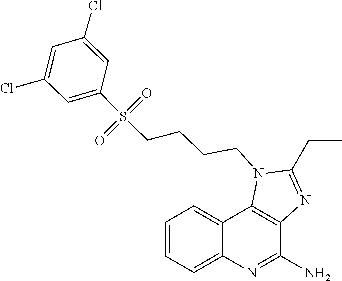 [SMILES: CCc1nc2c(N)nc3ccccc3c2n1CCCCS(=O)(=O)c1cc(Cl)cc(Cl)c1] |
| N-(2-{2-[4-amino-2- (ethoxymethyl)-1H- imidazo[4,5- c]quinolin-1- yl]ethoxy}ethyl)-n′- cyclohexylurea | 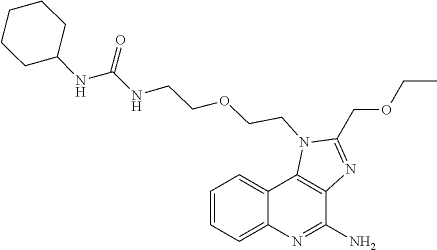 [SMILES: CCOCc1nc2c(N)nc3ccccc3c2n1CCOCCNC(=O)NC1CCCCC1] |
| N-{3-[4-amino-2- (ethoxymethyl)-1H- imidazo[4,5- c]quinolin-1-yl]propyl}-n′- (3-cyanophenyl)thiourea | 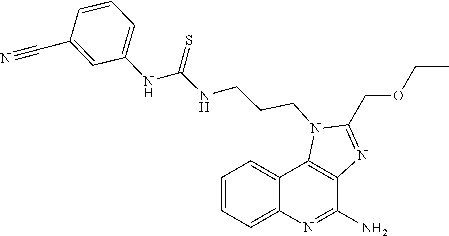 [SMILES: CCOCc1nc2c(N)nc3ccccc3c2n1CCCNC(=S)Nc1cccc(C#N)c1] |
| N-[3-(4-amino-2-butyl-1H- imidazo[4,5-c]quinolin-1- yl)-2,2- dimethylpropyl]benzamide | 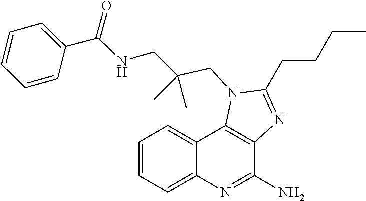 [SMILES: CCCCc1nc2c(N)nc3ccccc3c2n1CC(C)(C)CNC(=O)c1ccccc1] |
| 2-butyl-1-[3- (methylsulfonyl)propyl]- 1H- imidazo[4,5-c]quinolin-4- amine | 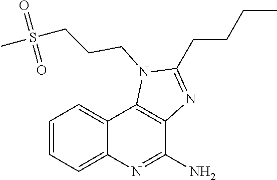 [SMILES: CCCCc1nc2c(N)nc3ccccc3c2n1CCCS(C)(=O)=O] |
| N-{2-[4-amino-2- (ethoxymethyl)-1H- imidazo[4,5- c]quinolin-1-yl]-1,1- dimethylethyl}-2- ethoxyacetamide | 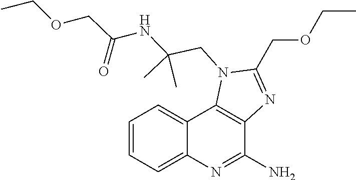 [SMILES: CCOCC(=O)NC(C)(C)Cn1c(COCC)nc2c(N)nc3ccccc3c21] |
| 1-[4-amino-2- ethoxymethyl-7-(pyridin-4- yl)-1H- imidazo[4,5-c]quinolin-1- yl]-2-methylpropan-2-ol | 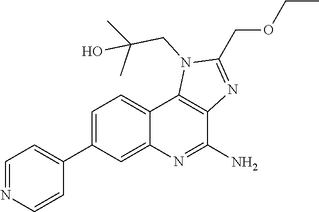 [SMILES: CCOCc1nc2c(N)nc3cc(-c4ccncc4)ccc3c2n1CC(C)(C)O] |
| 1-[4-amino-2- (ethoxymethyl)-7-(pyridin- 3-yl)-1H- imidazo[4,5-c]quinolin-1- yl]-2-methylpropan-2-ol | 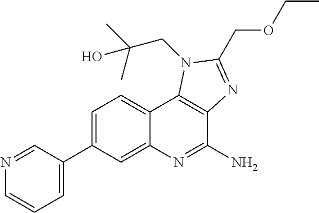 [SMILES: CCOCc1nc2c(N)nc3cc(-c4cccnc4)ccc3c2n1CC(C)(C)O] |
| N-{3-[4-amino-1-(2- hydroxy-2-methylpropyl)-2- (methoxyethyl)-1H- imidazo[4,5-c]quinolin-7- yl]phenyl}methanesulfonamide | 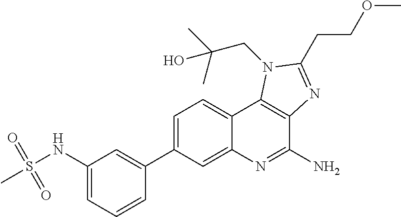 |
| 1-[4-amino-7-(5- hydroxymethylpyridin-3- yl)-2-(2- methoxyethyl)-1H- imidazo[4,5-c]quinolin-1- yl]-2- methylpropan-2-ol | 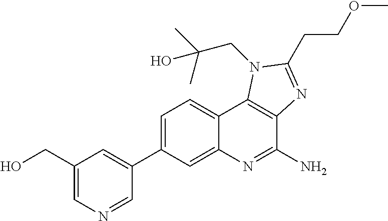 [SMILES: COCCc1nc2c(N)nc3cc(-c4cncc(CO)c4)ccc3c2n1CC(C)(C)O] |
| 3-[4-amino-2- (ethoxymethyl)-7-(pyridin- 3-yl)-1H- imidazo[4,5-c]quinolin-1- yl]propane-1,2-diol | 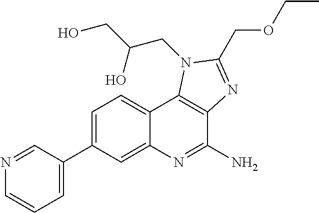 [SMILES: CCOCc1nc2c(N)nc3cc(-c4cccnc4)ccc3c2n1CC(O)CO] |
| 1-[2-(4-amino-2- ethoxymethyl-1H- imidazo[4,5- c]quinolin-1-yl)-1,1- dimethylethyl]-3-propylurea | 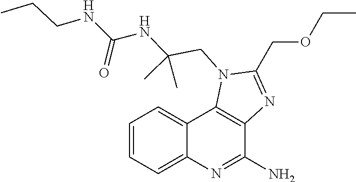 [SMILES: CCCNC(=O)NC(C)(C)Cn1c(COCC)nc2c(N)nc3ccccc3c21] |
| 1-[2-(4-amino-2- ethoxymethyl-1H- imidazo[4,5- c]quinolin-1-yl)-1,1- dimethylethyl]-3- cyclopentylurea | 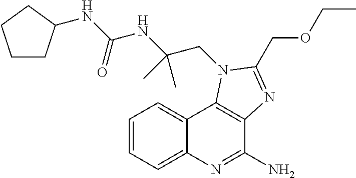 [SMILES: CCOCc1nc2c(N)nc3ccccc3c2n1CC(C)(C)NC(=O)NC1CCCC1] |
| 1-[(2,2-dimethyl-1,3- dioxolan-4-yl)methyl]-2- (ethoxymethyl)-7-(4- hydroxymethylphenyl)-1H- imidazo[4,5-c]quinolin-4- amine | 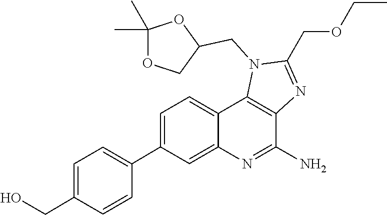 [SMILES: CCOCc1nc2c(N)nc3cc(-c4ccc(CO)cc4)ccc3c2n1CC1COC(C)(C)O1] |
| 4-[4-amino-2- ethoxymethyl-1-(2- hydroxy-2- methylpropyl)-1H- imidazo[4,5-c]quinolin-7- yl]-N- methoxy-N- methylbenzamide | 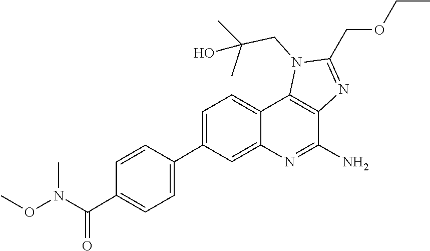 [SMILES: CCOCc1nc2c(N)nc3cc(-c4ccc(C(=O)N(C)OC)cc4)ccc3c2n1CC(C)(C)O] |
| 2-ethoxymethyl-N1- isopropyl-6,7,8,9- tetrahydro-1H- imidazo[4,5-c]quinoline- 1,4-diamine | 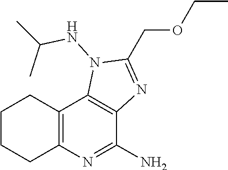 [SMILES: CCOCc1nc2c(N)nc3c(c2n1NC(C)C)CCCC3] |
| 1-[4-amino-2-ethyl-7- (pyridin-4-yl)-1H- imidazo[4,5- c]quinolin-1-yl]-2- methylpropan-2-ol | 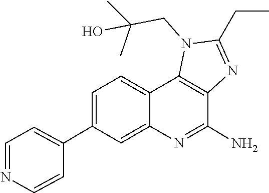 [SMILES: CCc1nc2c(N)nc3cc(-c4ccncc4)ccc3c2n1CC(C)(C)O] |
| N-[4-(4-amino-2-ethyl-1H- imidazo[4,5-c]quinolin-1- yl)butyl]methanesulfonamide | 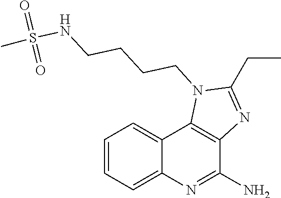 [SMILES: CCc1nc2c(N)nc3ccccc3c2n1CCCCNS(C)(=O)=O] |
| N-[4-(4-amino-2-butyl-1H- imidazo[4,5- c][1,5]naphthyridin-1- yl)butyl]-n′-cyclohexylurea | 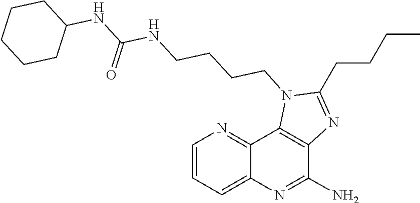 [SMILES: CCCCc1nc2c(N)nc3cccnc3c2n1CCCCNC(=O)NC1CCCCC1] |
| 3M-34240 | 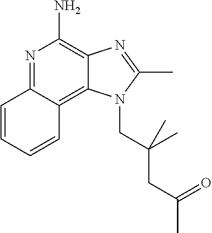 [SMILES: CC(=O)CC(C)(C)Cn1c(C)nc2c(N)nc3ccccc3c21] |
| 3M-052 | 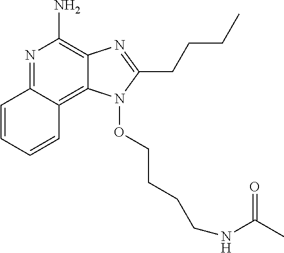 [SMILES: CCCCc1nc2c(N)nc3ccccc3c2n1OCCCCNC(C)=O] |
| 3M-854A | 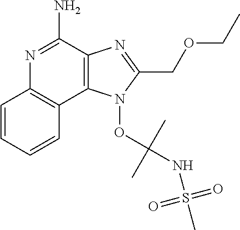 [SMILES: CCOCc1nc2c(N)nc3ccccc3c2n1OC(C)(C)NS(C)(=O)=O] |

Preferably in some embodiments, the immunotherapeutic is Resiquimod or Imiquimod.

In some embodiments, the immunotherapeutic is a TLR modulator (e.g., TLR7 and/or TLR8 agonist) that is represented by structure of Formula (II):

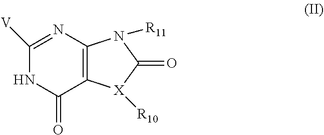

wherein V is —NR₆R₇, wherein each of R₆ and R₇ is independently hydrogen, alkyl, alkenyl, alkoxy, alkylamino, dialkylamino, alkylthio, arylthio, -alkyl-hydroxyl, -alkyl-C(O)—O—R₉, -alkyl-C(O)—R₉, or alkyl-O—C(O)—R₉, wherein R₉ is hydrogen, alkyl, alkenyl, hydrogen, or haloalkyl;
R₁₀ and R₁₁ are independently hydrogen, alkyl, alkenyl, aryl, haloalkyl, heteroaryl, heterocyclyl, or cycloalkyl, each of which is optionally substituted by one or more substituents selected from the group consisting of hydroxyl, alkoxy, alkyl, alkenyl, alkynyl, halogen, —N(R₅)₂, -alkoxy-alkyl, -alkoxy-alkenyl, —C(O)-alkyl, —C(O)—O-alkyl, —C(O)—N(R₅)2, aryl, heteroaryl, —CO-aryl, and —CO-heteroaryl, wherein each R₅ is independently hydrogen, alkyl, haloalkyl, -alkyl-aryl, or alkyl-heteroaryl,
or a pharmaceutically acceptable salt or solvate thereof.

In some embodiments, the immunotherapeutic is a TLR modulator (e.g., TLR7 and/or TLR8 agonist) that is represented by structure of Formula (III):

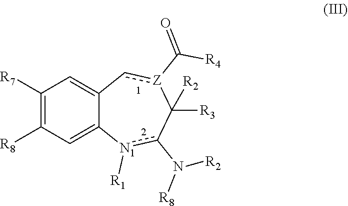

wherein [symbol] is a double bond or a single bond; R₂ and R₃ are independently selected from H and lower alkyl, or R₂ and R₃ are connected to form a saturated carbocycle having from 3 to 7 ring members; one of R₇ and R₈ is

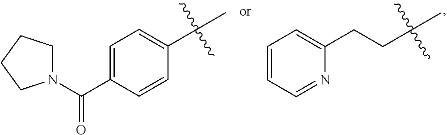 [SMILES: *c1ccc(C(=O)N2CCCC2)cc1 ; *CCc1ccccn1]

and the other is hydrogen; R₄ is —NR_{c}R_{d} or —OR₁₀; R_{c} and R_{d} are lower alkyl, where the alkyl is optionally substituted with one or more —OH; R₁₀ is alkyl, where the alkyl is optionally substituted with one or more —OH; Z is C and [symbol] is a double bond, or Z is N and [symbol] is a single bond; Rₐ and R_{b} are independently selected from H, alkyl, alkenyl, alkynyl, and R_{c}, wherein the alkyl is optionally substituted with one or more —OR₁₀, or Rₑ, Rₑ is selected from —NH₂, —NH(alkyl), and —N(alkyl)₂; R₁is absent when [symbol] is a double bond, or when [symbol] is a single bond, N₁—R₁ and one of Rₐ or R_{b} are connected to form a saturated, partially unsaturated, or unsaturated heterocycle having 5-7 ring members and the other of Rₐ or R_{b} may be hydrogen or absent as necessary to accommodate ring unsaturation; and at least one of the following A-D applies: A) R₇ is not hydrogen B) R₈ is not hydrogen and at least one of Rₐ, and R_{b} is not hydrogen; C) Z is N; or D) N₁—R₁ and one of Rₐ or R_{b} are connected to form a saturated, partially unsaturated, or unsaturated heterocycle having 5-7 ring members. US 20140088085A1, the disclosure of which is incorporated by references in its entirety.

In some embodiments, R₇ of the compound of Formula (III) is

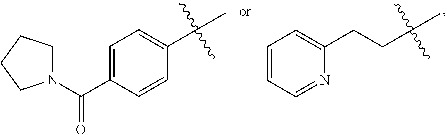 [SMILES: *c1ccc(C(=O)N2CCCC2)cc1 ; *CCc1ccccn1]

Additionally, at least one of Rₐ and R_{b} is not hydrogen in the compound of Formula (III), or, for example, one of Rₐ and R_{b} is alkyl and the other of Rₐ and R_{b} is hydrogen. Further, the alkyl of Formula (III) is substituted with R_{c}. in a different embodiment, both Rₐ and R_{b} are alkyl or, one of Rₐ and R_{b} is R_{c} and the other Rₐand R_{b} is hydrogen. For example, R₈of formula (III) is not hydrogen.

In some alternative embodiments, N₁ and one of Rₐ or R_{b} of Formula (III) are connected to form a saturated, partially unsaturated, or unsaturated heterocycle having 5-7 ring members and the other of Rₐ or R_{b} is hydrogen, or absent as necessary to accommodate ring unsaturation, where the ring is a 5 membered ring, or, for example, the ring is:

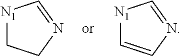

In some embodiments, at least one of R₂ and R₃ in the compound of Formula (III) is not hydrogen, or, for example, R₂ and R₃ are connected to form a saturated carbocycle, where the saturated carbocycle is cyclopropyl. Alternatively, Z is N in the compound of Formula (III).

In some embodiments, the TLR agonist or modulator has the structure of Formula (IV):

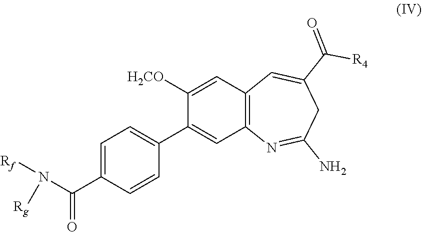

wherein R₄ is selected from —NR_{c}R_{d} and OR₁₀; R_{c} and R_{d} are lower alkyl, where the alkyl is optionally substituted with one or more OH; R₁₀is alkyl, where the alkyl is optionally substituted with one or more —O; R_{f}and R_{g} are lower alkyl or R_{f} and R_{g} together with the nitrogen atom to which they are attached form a saturated heterocyclic ring having 4-6 ring members. For example, R_{f} and R_{g} in the compound of Formula (IV), together with the nitrogen atom to which they are attached form a saturated heterocyclic ring, where the heterocyclic ring is pyrrolidine.

In some alternative embodiments, R₄ of either Formula (III) or Formula (IV) is —OR₁₀, where R₁₀ is alkyl or is ethyl. In another embodiment, R₄ of either Formula (III) or Formula (IV) is —NR_{c}R_{d}, where both are alkyl or both are propyl. Moreover, in certain embodiments, at least one of R_{c} or R_{d} is alkyl substituted with one —OH or at least one of R_{c} and R_{d} is

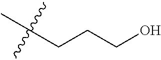 [SMILES: *CCCO]

and the remaining R_{c} or R_{d} is propyl.

In some alternative embodiments, the TLR agonist is a compound selected from

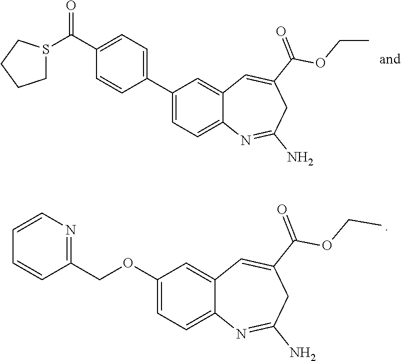

Alternatively, the compound is selected from

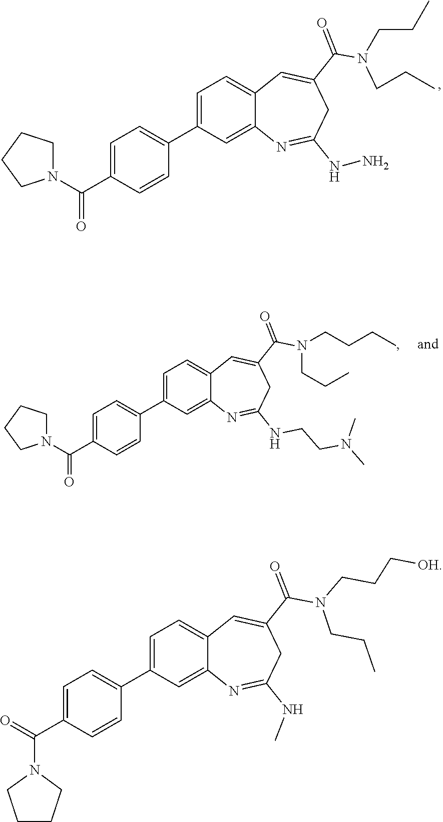 [SMILES: CCCN(CCC)C(=O)C1=Cc2ccc(-c3ccc(C(=O)N4CCCC4)cc3)cc2N=C(NN)C1 ; CCCCN(CCC)C(=O)C1=Cc2ccc(-c3ccc(C(=O)N4CCCC4)cc3)cc2N=C(NCCN(C)C)C1 ; CCCN(CCCO)C(=O)C1=Cc2ccc(-c3ccc(C(=O)N4CCCC4)cc3)cc2N=C(NC)C1]

In some alternative embodiments, the TLR agonist compound is either

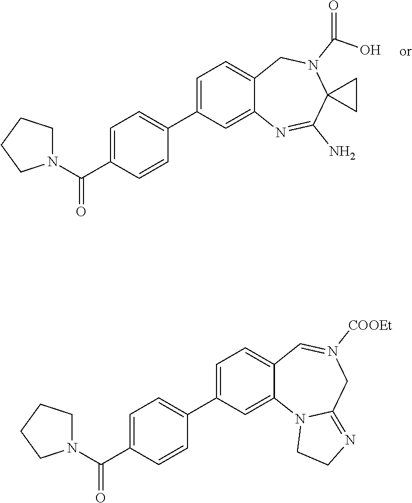

In some alternative embodiments, the TLR agonist is a compound selected from

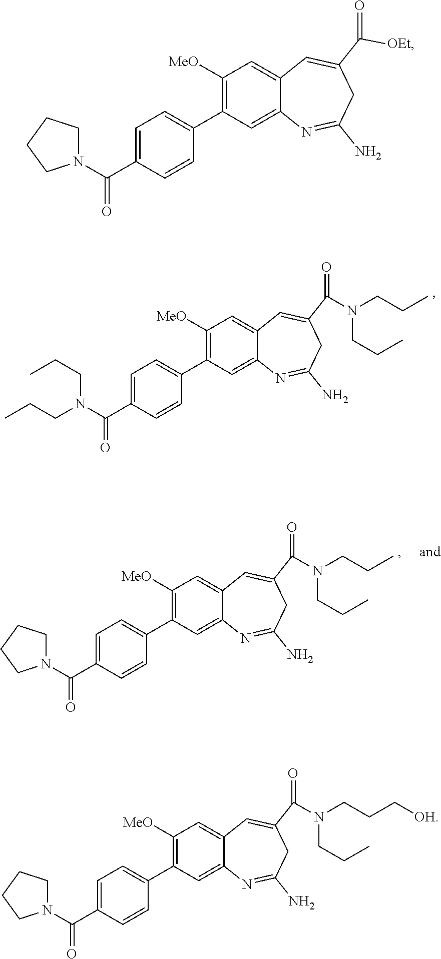 [SMILES: CCOC(=O)C1=Cc2cc(OC)c(-c3ccc(C(=O)N4CCCC4)cc3)cc2N=C(N)C1 ; CCCN(CCC)C(=O)C1=Cc2cc(OC)c(-c3ccc(C(=O)N(CCC)CCC)cc3)cc2N=C(N)C1 ; CCCN(CCC)C(=O)C1=Cc2cc(OC)c(-c3ccc(C(=O)N4CCCC4)cc3)cc2N=C(N)C1 ; CCCN(CCCO)C(=O)C1=Cc2cc(OC)c(-c3ccc(C(=O)N4CCCC4)cc3)cc2N=C(N)C1]

In some alternative embodiments, the TLR agonist is

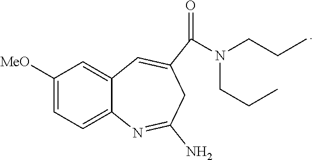 [SMILES: CCCN(CCC)C(=O)C1=Cc2cc(OC)ccc2N=C(N)C1]

In some alternative embodiments, the TLR agonist is a compound selected from:

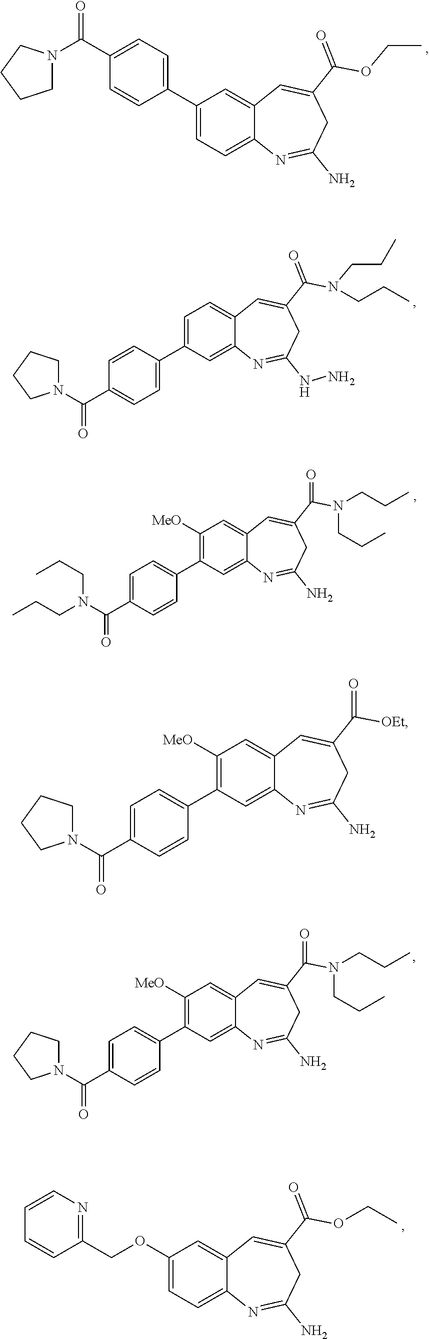

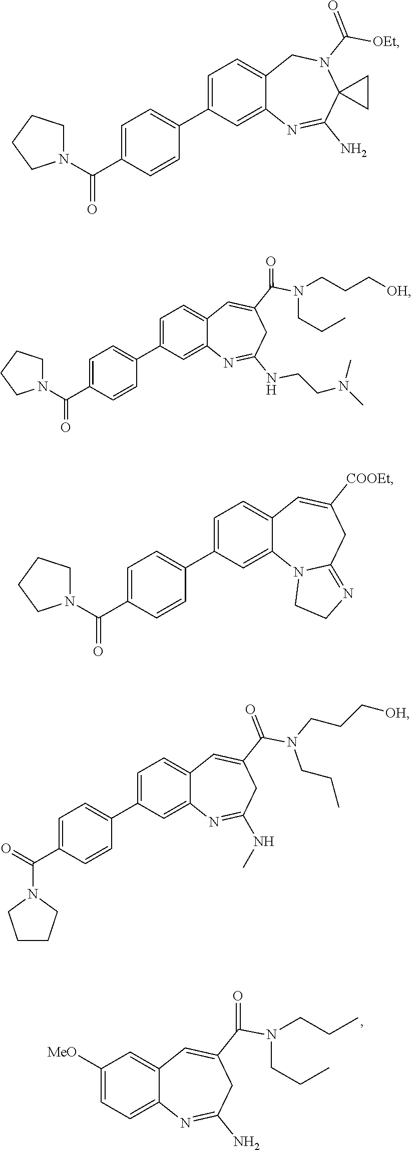 [SMILES: CCOC(=O)N1Cc2ccc(-c3ccc(C(=O)N4CCCC4)cc3)cc2N=C(N)C12CC2 ; CCCN(CCCO)C(=O)C1=Cc2ccc(-c3ccc(C(=O)N4CCCC4)cc3)cc2N=C(NCCN(C)C)C1 ; CCOC(=O)C1=Cc2ccc(-c3ccc(C(=O)N4CCCC4)cc3)cc2N2CCN=C2C1 ; CCCN(CCCO)C(=O)C1=Cc2ccc(-c3ccc(C(=O)N4CCCC4)cc3)cc2N=C(NC)C1 ; CCCN(CCC)C(=O)C1=Cc2cc(OC)ccc2N=C(N)C1]

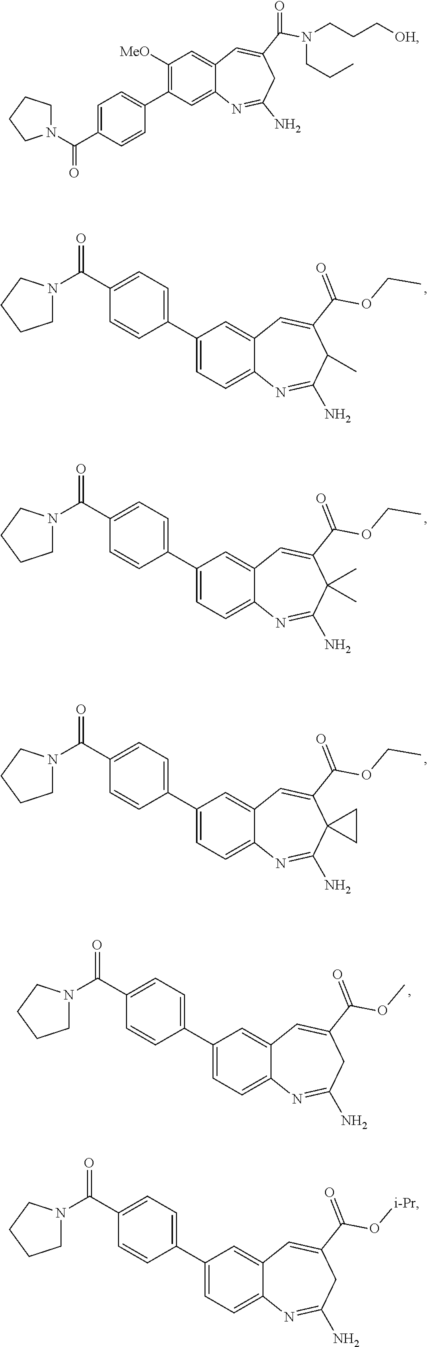 [SMILES: CCCN(CCCO)C(=O)C1=Cc2cc(OC)c(-c3ccc(C(=O)N4CCCC4)cc3)cc2N=C(N)C1 ; CCOC(=O)C1=Cc2cc(-c3ccc(C(=O)N4CCCC4)cc3)ccc2N=C(N)C1C ; CCOC(=O)C1=Cc2cc(-c3ccc(C(=O)N4CCCC4)cc3)ccc2N=C(N)C1(C)C ; CCOC(=O)C1=Cc2cc(-c3ccc(C(=O)N4CCCC4)cc3)ccc2N=C(N)C12CC2 ; COC(=O)C1=Cc2cc(-c3ccc(C(=O)N4CCCC4)cc3)ccc2N=C(N)C1 ; CC(C)OC(=O)C1=Cc2cc(-c3ccc(C(=O)N4CCCC4)cc3)ccc2N=C(N)C1]

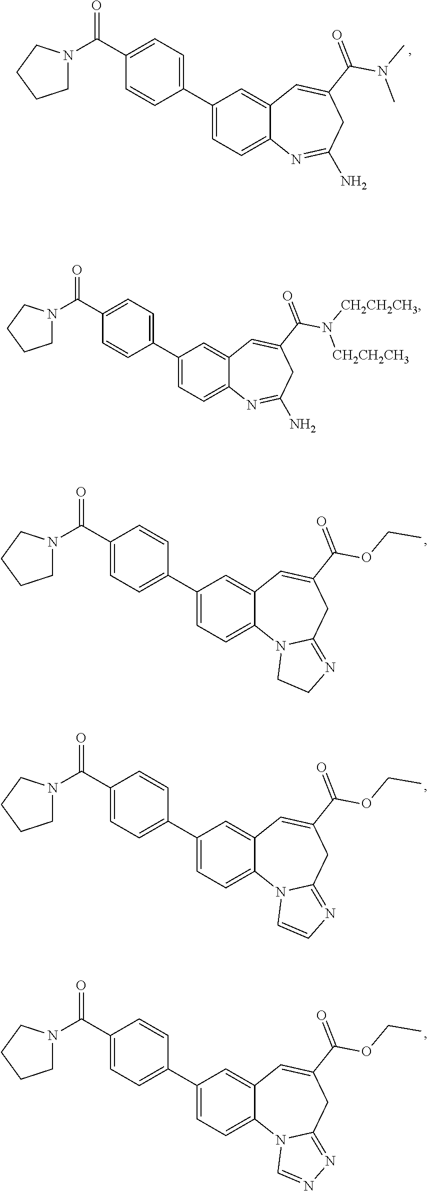

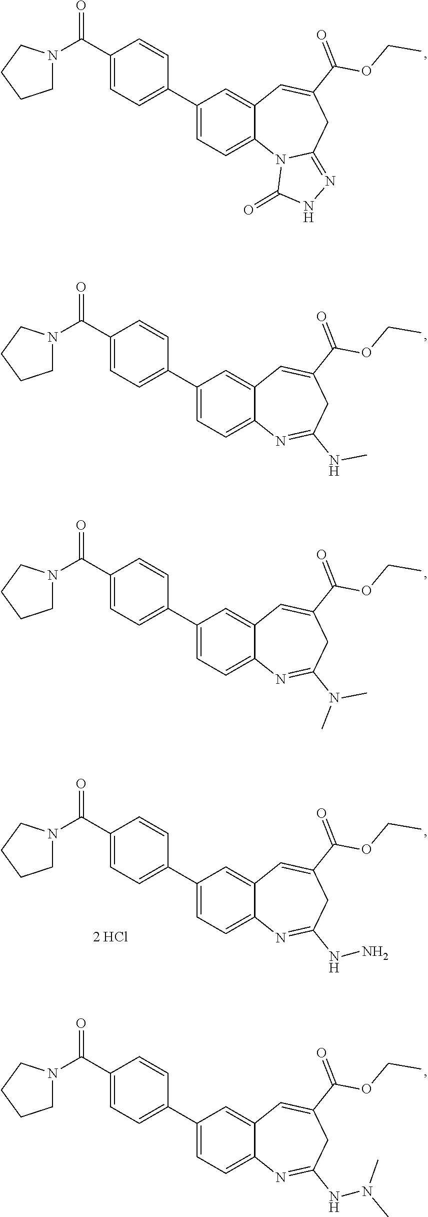 [SMILES: CCOC(=O)C1=Cc2cc(-c3ccc(C(=O)N4CCCC4)cc3)ccc2-n2c(n[nH]c2=O)C1 ; CCOC(=O)C1=Cc2cc(-c3ccc(C(=O)N4CCCC4)cc3)ccc2N=C(NC)C1 ; CCOC(=O)C1=Cc2cc(-c3ccc(C(=O)N4CCCC4)cc3)ccc2N=C(N(C)C)C1 ; CCOC(=O)C1=Cc2cc(-c3ccc(C(=O)N4CCCC4)cc3)ccc2N=C(NN)C1 ; CCOC(=O)C1=Cc2cc(-c3ccc(C(=O)N4CCCC4)cc3)ccc2N=C(NN(C)C)C1]

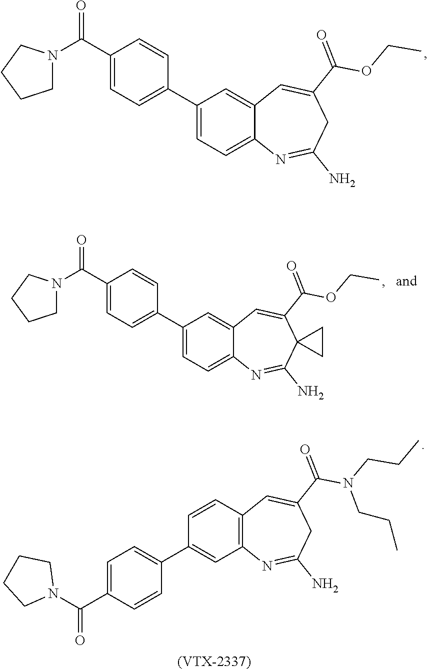

In some embodiments, the immunotherapeutic is a TLR modulator (e.g., TLR7 and/or TLR8 agonist) that is represented by structure of Formula (V):

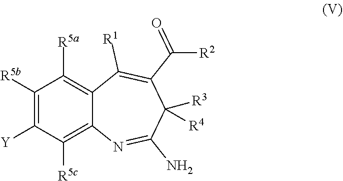

and metabolites, solvates, tautomers, and prodrugs thereof, wherein:

Y is CF₂CF₃, CF₂CF₂R⁶, or an aryl or heteroaryl ring, wherein said acyl and heteroaryl rings are substituted with one or more groups independently selected from alkenyl, alkynyl, Br, CN, OH, NR⁶R⁷, C(═O)R⁸, NR⁶SO₂R⁷, (C₁-C₆alkyl)amino, R⁶OC(═O)CH═CH₂—, SR⁶ and SO₂R⁶, and wherein the aryl and heteroaryl rings arc optionally further substituted with one or more groups independently selected from F, Cl, CF₃, HCF₂O—, alkyl, heteroalkyl and ArO—;

R¹, R³ and R⁴ are independently selected from H, alkyl, alkenyl, alkynyl, heteroalkyl, cycloalkyl, cycloalkenyl, heterocycloalkyl, aryl and heteroaryl, wherein the alkyl, alkenyl, alkynyl, heteroalkyl, cycloalkyl, cycloalkenyl, heterocycloalkyl, acyl and heteroaryl are optionally substituted with one or more groups independently selected from alkyl, alkenyl, alkynyl, F, Cl; Br, I, CN, OR⁶, NR⁶R⁷, C(═O)R⁶, C(═O)OR⁶, OC(═O)R⁶, C(═O)NR⁶R⁷, (C₁-C₆alkyl)amino, R⁶OC(^O)CH═CH₂—, NR⁶SO₂R⁷, SR⁶ and SO₂R⁶,

or R³ and R⁴ together with the atom to which they are attached form a saturated or partially unsaturated carbocyclic ring, wherein the carbocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆alkyl)amino, CH₃OCH₂O—,R⁶OC(═O)CH═CH₂—, NR⁶SO₂R⁷, SR⁶ and SO₂R⁶;

R² and R⁸ are independently selected from H, OR⁶, NR⁶R⁷, alkyl, alkenyl, alkynyl, heteroalkyl, cycloalkyl, cycloalkenyl, heterocycloalkyl, aryl and heteroaryl, wherein the alkyl, alkenyl, alkynyl, heteroalkyl, cycloalkyl, cycloalkenyl, heterocycloalkyl, aryl and heteroaryl are optionally substituted with one or more groups independently selected from alkyl, alkenyl, alkynyl, F, Cl, Br5 CN, OR⁶, NR⁶R⁷, C(═O)R⁶, C(═O)OR⁶, OC(═O)R⁶, C(^O)NR⁶R⁷, (C₁-C₆alkyl)amino, CH₃OCH₂O—, R⁶OC(═O)CH═CH₂—, NR⁶SO₂R⁷, SR⁶ and SO₂R⁶;

R^{5a}, R^{5b}, and R^{5c} are independently H, F, Cl, Br, I₅OMe₅CH₃, CH₂F₅CHF₂or CF₃; and

R⁶ and R⁷ are independently selected from H₅ alkyl, alkenyl, alkynyl, heteroalkyl, cycloalkyl, cycloalkenyl, heterocycloalkyl, aryl and lieteroaryl, wherein said alkyl, alkenyl, alkynyl, heteroalkyl, cycloalkyl, cycloalkenyl, heterocycloalkyl, aryl and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆alkyl)amino, CH₃OCH₂O—, R⁶OC(^O)CH═CH₂—, NR⁶SO₂R⁷, SR⁶ and SO₂R⁶,

or R⁶ and R⁷ together with the atom to which they are attached form a saturated or partially unsaturated heterocyclic ring, wherein said heterocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆alkyl)amino, CH₃OCH₂O—, R⁶OC(═O)CH═CH₂—, NR⁶SO₂R⁷, SR⁶ and SO₂R⁶. in certain embodiments, R¹, R³ and R⁴ are each hydrogen. In certain embodiments, R^{5a}, R^{5b} and R^{5c} are each hydrogen. WO 2007024612 A2, the disclosure of which is incorporated by reference in its entirety.

In some embodiments of the compound of Formula (V), R² is OR⁶. In some embodiments, R⁶ is alkyl, such. as (C₁₋₄)alkyl. In particular embodiments, R⁶ is ethyl.

In some embodiments of the compound of Formula (V), R² is NR⁶R⁷. In some embodiments, R⁶ and R⁷ are independently H, alkyl, such as (1-6C)alkyl, or heteroalkyl, such as (C₁₋₄alkoxy C₂₋₄)alkyl. In particular embodiments, R⁶ and R⁷ are independently H, ethyl, propyl, or CH₂CH₂OCH₃. In some embodiments of the compound of Formula V, Y is amyl, such as phenyl. In some embodiments, the awl is substituted with C(═O)R⁸, such as in para-R⁸C(═O)phenyl. In some embodiments, R⁸ is OR⁶, NR⁶R⁷ or heterocycloalkyl. In some embodiments, R⁶ and R⁷ are independently H or alkyl, such as (C₁₋₆)alkyl. In some other embodiments, R⁶ and R⁷ together with the nitrogen atom to which they are attached form a 4-6 membered azacycloalkyl ring, such as pyrrolidinyl. In some embodiments, Y is

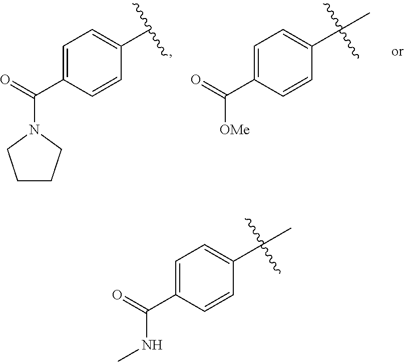 [SMILES: *c1ccc(C(=O)N2CCCC2)cc1 ; *c1ccc(C(=O)OC)cc1 ; *c1ccc(C(=O)NC)cc1]

In some embodiments of the compound of Formula (V), Y is CF₂CF₃.

In some embodiments, the immunotherapeutic is a TLR modulator (e.g., TLR8 agonist) that is represented by structure of formula (VI):

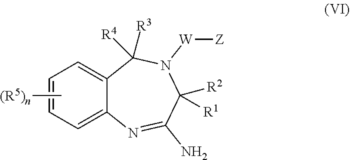

and metabolites, solvates, tautomers, and pharmaceutically acceptable prodrugs and salts thereof, wherein:

Z is H, alkyl, alkenyl, alkynyl, heteroalkyl, cycloalkyl, heterocycloalkyl, aryl, heteroaryl, OR⁶ or NR⁶R⁷, wherein said alkyl, alkenyl, alkynyl; heteroalkyl, cycloalkyl, heterocycloalkyl, aryl and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆alkyl)amino, CH₃OCH₂O—, R⁶OCC═O)CH═CH₂—, NR⁶SO₂R⁷, SR⁶ and SO₂R⁶;

R², R³ and R³ are independently selected from H, alkyl, alkenyl, alkynyl, heteroalkyl, cycloalkyl, cycloalkenyl, heterocycloalkyl, aryl and heteroaryl, wherein said alkyl, alkenyl, alkynyl, heteroalkyl, cycloalkyl, cycloalkenyl, heterocycloalkyl, aryl, and heteroaryl are optionally substituted with one or more groups independently selected from alkyl, alkenyl, alkynyl, F, Cl, Br, I, CN, OR⁶, NR⁶R⁷, CC═O)R⁶, C(═O)OR⁶, OC(═O)R⁶, CC═O)NR⁶R⁷, (C₁-C₆alkyl)amino, CH₃OCH₂O—, R⁶OCC═O)CH═CH₂—, NR⁶SO₂R⁷, SR⁶ and SO₂R⁶,

or R¹ and R² together with the atom to which they are attached form a saturated or partially unsaturated carbocyclic ring, wherein said carbocyclic ring is optionally substituted with one or more groups independently selected from alkyl, alkenyl, alkynyl, F, Cl, Br, I, CN, OR⁶, NR⁶R⁷, C(═O)R⁶, CC═O)OR⁶, OC(═O)R⁶, CC═O)NR⁶R⁷, CCi-C₆alkyl)amino, CH₃OCH₂O—, R⁶OCC═O)CH═CH₂—, NR⁶SO₂R⁷, SR⁶ and SO₂R⁶;

or R³ and R⁴ together are oxo;

each R³ is independently selected from H, F, CI, Br, I, OMe, CH₃, CH₂F, CHF₂, CF₃ and CF₂CF₃;

R⁶ and R⁷ are independently selected from H, alkyl, alkenyl, alkynyl, heteroalkyl, cycloalkyl, cycloalkenyl, heterocycloalkyl, aryl, and heteroaryl, wherein said alkyl, alkenyl, alkynyl, heteroalkyl, cycloalkyl, cycloalkenyl, heterocycloalkyl, aryl, and heteroaryl are optionally substituted with one or more groups independently selected from alkyl, alkenyl, alkynyl, F, CL Br, I, CN, OR⁶, NR⁶R⁷, CC═O)R⁶, C(=0)0R⁶, 0C(=0)R⁶, CC═O)NR⁶R⁷, (C₁-C₆alkyl)amino, CH₃OCH₂O—, R⁶OC(═O)CH═CH₂—, NR⁶SO₂R⁷, SR⁶ and SO₂R⁶;

or R⁶ and R⁷ together with the atom to which they are attached form a saturated or partially unsaturated heterocyclic ring, wherein the heterocyclic ring is optionally substituted with one or more groups independently selected from alkyl, alkenyl, alkynyl, F, Cl, Br, I, CN, OR⁶, NR⁶R⁷, CC═O)R⁶, C(=0)0R⁶, 0C(═O)R⁶, C(═O)NR⁶R⁷, (C₁-C₆alkyl)amino, CH₃OCH₂O—, R⁶OC(═O)CH═CH₂—, NR⁶SO₂R⁷, SR⁶ and SO₂R⁶; and n is O, 1, 2, 3 or 4. WO2007040840A2, the disclosure of which is incorporated by reference in its entirety.

In some embodiments, the immunotherapeutic is a TLR modulator (e.g.,TLR7 and/or 8 agonist) that is represented by structure of Formula (VI):

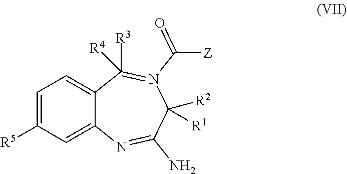

and metabolites, solvates, tautomers, and pharmaceutically acceptable salts and prodrugs thereof, wherein:

Z is H, alkyl, alkenyl, alkynyl, heteroalkyl, cycloalkyl, heterocycloalkyl, aryl, heteroaryl, OR⁶ or NR⁶R⁷, wherein the alkyl, alkenyl, alkynyl, heteroalkyl, cycloalkyl, heterocycloalkyl, aryl and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, C₆alkyl)amino, CH₃OCH₂O—, R⁶OCC═O)CH═CH₂—, NR⁶SO₂R⁷, SR⁶ and SO₂R⁶,

R¹, R², R³ and R⁴ are independently selected from H, alkyl, alkenyl, alkynyl, heteroalkyl, . cycloalkyl, cycloalkenyl, heterocycloalkyl, aryl and heteroaryl, wherein said alkyl, alkenyl, alkynyl, heteroalkyl, cycloalkyl, cycloalkenyl, heterocycloalkyl, aryl, and heteroaryl are optionally substituted with one or more groups independently selected from alkyl, alkenyl, alkynyl, F, Cl, Br, I₉CN, OR⁶, NR⁶R⁷, C(=0)R⁶, C(═O)OR⁶, OC(═O)R⁶, C(═O)NR⁶R⁷, (C₁-C₆alkylamino, CH₃OCH₂O—, R⁶OCC═O)CH═CH₂—, NR⁶SO₂R₇, SR⁶ and SO₂R⁶,

or R¹ and R² together with the atom to which they are attached form a saturated or partially unsaturated carbocyclic ring, wherein said carbocyclic ring is optionally substituted with one or more groups independently selected from alkyl, alkenyl, alkynyl, F, Cl, Br, I, CN, OR⁶, NR⁶R⁷, C(=0)R⁶, C(═O)OR⁶, OC(═O)R⁶, C(═O)NR⁶R⁷, alkyl)amino, CH₃OCH₂O—, R⁶OC(═O)CH═CH₂—, NR⁶SO₂R⁷, SR⁶ and SO₂R⁶,

or R³ and R⁴ together are oxo;

R³ is H, F, Cl, Br, I, OMe, CH₃, CH₂F, CHF₂, CF₃ or CF₂CF₃;

R⁶ and R⁷ are independently selected from I-I, alkyl, alkenyl, alkynyl, heteroalkyl, cycloalkyl, cycloalkenyl, heterocycloalkyl, aryl, and heteroaryl, wherein said alkyl, alkenyl, alkenyl, heteroalkyl, cycloalkyl, cycloalkenyl, heterocycloalkyl, aryl, and heteroaryl are optionally substituted with one or more groups independently selected from alkyl, alkenyl. alkynyl, F, Cl, Br, I, CN, OR⁶, NR⁶R⁷, C(═O)R⁶, C(═O)OR, OC(═O)R⁶, C(═O)NR⁶R⁷, (C₁-C₆alkyl)amino₅ CH₃OCH₂O—, R⁶OC(═O)CH═CH₂—, NR⁶SO₂R⁷, SR⁶ and SO₂R⁶; or R⁶ and R⁷ together with the atom to which they are attached form a saturated or partially unsaturated heterocyclic ring, wherein said heterocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆alkyl)ammo, CH₃OCH₂O—, R⁶OC(═O)CH═CH₂—, NR⁶SO₂R⁷, SR⁶ and SO₂R⁶; and

n is 0, 1, 2, 3 or 4.

In some embodiments, Z is OR⁶. In some embodiments, R⁶ is alkyl, such as (1-6C)alkyl. In particular embodiments, R⁶ is ethyl, propyl, isopropyl or isobutyl.

In some embodiments, Z is NR⁶R⁷. In some embodiments, R⁶ and R⁷ are independently H or alkyl, such as (1-6C)alkyl. In some embodiments, R₆ and R⁷ are ethyl. In some embodiments, n is O or 1.

In some embodiments, R⁵ is C F₂CF₃. In certain embodiments, R³ is H or alkyl, such as (1-4C)alkyl, and R⁴ is H. In certain embodiments, R is alkyl, such as (1-4C)alkyl. In some embodiments, R is methyl. In other particular embodiments, R³ is H. In some embodiments, R is H or alkyl, such as (1-4C)alkyl and R is H. In some embodiments, R¹ is alkyl. In some embodiments, R¹ is methyl. In some particular embodiments, R¹ is H.

In some embodiments, the TLR7 and/or TLR8 agonist that is represented by structure of Formula (XV):

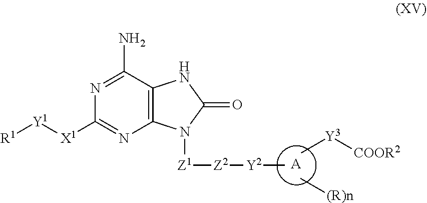

wherein ring A represents a 6-10 membered aromatic carbocyclic ring or a 5-10 membered heteroaromatic ring;
R represents a halogen atom, an alkyl group, a hydroxyalkyl group, a haloalkyl group, an alkoxy group, a hydroxyalkoxy group, a haloalkoxy group, amino group, an alkylamino group, a dialkylamino group, or a 4-7 membered cyclic group containing in the ring 1-2 hetero atoms selected from 1-2 nitrogen atoms and optionally 0-1 oxygen atom or 0-1 sulfur atom;
n represents an integer of 0-2, and when n is 2, the Rs may be the same or different;
Z¹ represents a substituted or unsubstituted alkylene group or a substituted or unsubstituted cycloalkylene group;
X² represents oxygen atom, sulfur atom, SO₂, NR⁵, CO, CONR⁵, NR⁵CO, SO₂NR⁵, NR⁵SO₂, NR⁵CONR⁶ or NR⁵CSNR⁶ (in which R⁵ and R⁶ are each independently hydrogen atom, a substituted or unsubstituted alkyl group, or a substituted or unsubstituted cycloalkyl group);
Y¹, Y² and Y³ represent each independently a single bond or an alkylene group;
X¹ represents oxygen atom, sulfur atom, SO₂, NR⁴ (wherein R⁴ is hydrogen atom or an alkyl group) or a single bond;
R² represents hydrogen atom, a substituted or unsubstituted alkyl group, a substituted or unsubstituted alkenyl group, a substituted or unsubstituted alkynyl group or a substituted or unsubstituted cycloalkyl group; and
R¹ represents hydrogen atom, hydroxy group, an alkoxy group, an alkoxycarbonyl group, a haloalkyl group, a haloalkoxy group, a substituted or unsubstituted aryl group, a substituted or unsubstituted heteroaryl group or a substituted or unsubstituted cycloalkyl group. The linker is linked to one of the possible linking site of the angonist, such as to —NH₂.
In some embodiments, R¹ represents hydrogen, hydroxyl, or a C₁-C₆alkoxy, C₂-C₅alkoxycarbonyl, C₁-C₆haloalkyl, C₁-C₆ haloalkoxy, C₆-C₁₀ aryl, C₅-C₁₀heteroaryl or C₃-C₈cycloalkyl group, each group being optionally substituted by one or more substituents independently selected from halogen, hydroxyl, a C₁-C₆alkyl, C₁-C₆haloalkyl, C₁-C₆alkoxy, C₁-C₆haloalkoxy, C₂-C₅ alkoxycarbonyl, amino (NH₂), (mono)-C₁-C₆alkylamino and (di)-C₁-C₆alkylamino group;
Y¹ represents a single bond or C₁-C₆alkylene;
X¹ represents a single bond, an oxygen, sulphur atom, sulphonyl (SO₂) or NR³;
Z¹ represents a C₂-C₆alkylene or C₃-C₈ cycloalkylene group, each group being optionally substituted by at least one hydroxyl;
X² represents NR⁴;
Y² represents a single bond or C₁-C₆alkylene;
Y³ represents a single bond or C₁-C₆alkylene;
n is an integer 0, 1 or 2;
R represents halogen or a C₁-C₆alkyl, C₁-C₆ hydroxyalkyl, C₁-C₆haloalkyl, C₁-C₆alkoxy, C₁-C₆hydroxyalkoxy, C₁-C₆haloalkoxy, amino (NH₂), (mono)-C₁-C₆alkylamino, (di)-C₁-C₆alkylamino group or a C₃-C₈saturated heterocyclic ring containing a ring nitrogen atom and optionally one or more further heteroatoms independently selected from nitrogen, oxygen and sulphur, the heterocyclic ring being optionally substituted by one or more substituents independently selected from halogen, hydroxyl, oxo, C₁-C₆alkyl, C₁-C₆alkoxy, C₂-C₅ alkylcarbonyl and C₂-C₅alkoxycarbonyl;
R² represents hydrogen or a C₁-C₆alkyl, C₂-C₆alkenyl, C₂-C₆alkynyl or C₃-C₈cycloalkyl group, each group being optionally substituted by one or more substituents independently selected from halogen, hydroxyl or a C₁-C₆alkoxy, a C₂-C₁₀ acyloxy, group selected from a C₂₋₅alkylcarbonyloxy group, a C₂-C₅ alkenylcarbonyloxy group, a C₂-C₅alkynylcarbonyloxy group, a C₆-C₉arylcarbonyloxy group and a C₅-C₉heteroarylcarbonyloxy group, each of which acyloxy groups may be optionally substituted by one or more substituents independently selected from halogen, hydroxyl, C₁-C₃ alkoxy and phenyl providing that the total number of carbon atoms in the acyloxy group does not exceed 10, amino (NH₂), (mono)-C₁-C₆alkylamino, (di)-C₁-C₆alkylamino group and a C₃-C₈saturated heterocyclic ring containing a ring nitrogen atom and optionally one or more further heteroatoms independently selected from nitrogen, oxygen and sulphur, the heterocyclic ring in turn being optionally substituted by one or more substituents independently selected from halogen, hydroxyl, oxo, C₁-C₆alkyl, C₁-C₆alkoxy, C₂-C₅alkylcarbonyl and C₂-C₅ alkoxycarbonyl group;
R³ represents hydrogen or C₁-C₆alkyl;
R⁴ represents CO₂R⁵, SO₂R⁵, COR^{S}, SO₂NR⁶R⁷ and CONR⁶R⁷;
R⁵ independently represents
(i) 3- to 8-membered heterocyclic ring containing 1 or 2 heteroatoms selected from a ring group NR⁸, S(O)ₘ or oxygen, the 3- to 8-membered heterocyclic ring being optionally substituted by one or more substituents independently selected from halogen, hydroxyl or a C₁-C₆alkyl and C₁-C₆alkoxy group, or
(ii) a C₆-C₁₀aryl or C₅-C₁₀heteroaryl group, each of which may be optionally substituted by one or more substituents independently selected from halogen, cyano, C₁-C₆alkyl, C₁-C₃haloalkyl, carboxyl, S(O)ₘR⁹, OR¹⁰, CO₂R¹⁰, SO₂NR¹⁰R¹¹, CONR¹⁰R¹¹, NR¹⁰R¹¹, NR¹⁰SO₂R⁹, NR¹⁰CO₂R⁹, NR¹⁰COR⁹, or
(iii) a C₁-C₆alkyl, C₂-C₆alkenyl, C₂-C₆alkynyl or C₃-C₈cycloalkyl group, each of which may be optionally substituted by one or more substituents independently selected from halogen, CN, C₃-C₈cycloalkyl, S(O)ₚR¹², OR¹³, COR¹³, CO₂R¹³, SO₂NR¹³R¹⁴, CONR¹³R¹⁴, NR¹³R¹⁴, NR¹³SO₂R¹², NR¹³CO₂R¹², NR¹³COR¹², NR¹³SO₂R¹² or a C₆-C₁₀aryl or C₅-C₁₀heteroaryl group or a heterocyclic ring, the latter three groups may be optionally substituted by one or more substituents independently selected from C₁-C₆alkyl (optionally substituted by hydroxy, C₁-C₆alkoxy, C₁-C₆alkoxycarbonyl, amino, C₁-C₆alkylamino, di-C₁-C₆alkylamino, NH₂C(O)—, C₁-C₆alkyl NHC(O), di-C₁-C₆alkyl NC(O), —OCH₂CH₂OH, pyrrolidinyl, pyrrolidinylcarbonyl, furanyl, piperidyl, methylpiperidyl or phenyl), C₂-C₆alkenyl (optionally substituted by phenyl), halogen, hydroxy, cyano, carboxy, amino, C₁-C₆alkylamino, di-C₁-C₆alkylamino, NH₂C(O)—, C₁-C₆alkyl NHC(O)—, di-C₁-C₆alkyl NC(O), C₁-C₆alkoxycarbonyl, C₁-C₆alkylsulphonyl, C₁-C₆alkylcarbonylamino, C₁-C₆alkylcarbonylmethylamino, phenyl (optionally substituted by hydroxy, fluoro or methyl), pyrrolidinyl, pyridyl, piperidinyl, benzothiazolyl or pyrimidinyl;
R⁶ represents hydrogen or a C₁-C₆alkyl, C₂-C₆alkenyl, C₂-C₆alkynyl, C₃-C₈cycloalkyl group or heterocyclic ring, each of which may be optionally substituted by one or more substituents independently selected from halogen, hydroxyl, oxo, cyano, C₁-C₆alkyl, C₂-C₆alkenyl, C₂-C₆alkynyl, C₃-C₈cycloalkyl, OR¹⁵, S(O)_{q}R¹⁵, CO₂R¹⁶, COR¹⁶, NR¹⁶R¹⁷, CONR¹⁶R¹⁷, NR¹⁶COR¹⁷, NR¹⁶CO₂R¹⁵, SO₂NR¹⁶R¹⁷, NR¹⁶SO₂R¹⁵, or a C₆-C₁₀aryl or C₅-C₁₀heteroaryl group or heterocyclic ring, the latter three groups being optionally substituted by one or more substituents independently selected from, C₁-C₆alkyl, C₃-C₈cycloalkyl, halogen, S(O)_{q}R¹⁵, CO₂R¹⁶, COR¹⁶, hydroxy or cyano; and
R⁷ represents hydrogen, a C₁-C₆alkyl, C₂-C₆alkenyl, C₂-C₆alkynyl, or C₃-C₈cycloalkyl group, each group may be optionally substituted by one or more substituents independently selected from halogen, C₃-C₈cycloalkyl, a C₆-C₁₀aryl or C₅-C₁₀heteroaryl group, carboxy, cyano, OR¹⁵, hydroxy or NR¹⁸R¹⁹, or

R⁶ and R⁷ together with the nitrogen atom to which they are attached fowl a 3- to 8-membered saturated or partially saturated heterocyclic ring, optionally containing further heteroatoms or heterogroups selected from nitrogen, S(O). or oxygen, the heterocyclic ring, may be optionally substituted by one or more substituents independently selected from halogen, hydroxyl, carboxyl, cyano, OR²⁰, N²¹R₂₂, S(O)_{q}R²³, COR²⁴, CO₂R²⁴, NR²⁴R₂₅, CONR²⁴R²⁵, NR²⁴COR²⁵, NR²⁴CO₂R²³, SO₂NR²⁴R²⁵, NR²⁴SO₂R²³, C₆-C₁₀aryl , C₅-C₁₀heteroaryl group, heterocyclic ring, C₁-C₆alkyl, C₂-C₆alkenyl, C₂-C₆alkynyl or C₃-C₈cycloalkyl group, the latter seven groups being optionally substituted by one or more substituents independently selected from halogen, hydroxyl, oxo, cyano, OR²⁰, S(O)_{q}R²³, COR²⁴, CO₂R²⁴, NR²⁴R²⁵, CONR²⁴R²⁵, NR²⁴CO₂R²³, NR²⁴COR²⁵, SO₂NR²⁴R²⁵, NR²⁴SO₂R²³, a heterocyclic ring or a C₆-C₁₀aryl or C₅-C₁₀heteroaryl group, the latter three groups being optionally substituted by one or more substituents independently selected from C₁-C₆alkyl, halogen, hydroxy or cyano;
R⁸ represents hydrogen, CO₂R²⁶, COR²⁶, SO₂R²⁶, C₁-C₆alkyl or C₃-C₆cycloalkyl group, each group may be optionally substituted by one or more substituents independently selected from halogen, hydroxyl, and NR²⁷R²⁸;
R¹⁰, R¹¹, R¹⁶, R¹⁷, R¹⁸, R¹⁹, R²¹, R²², R²⁶, R²⁷ or R²⁸ each independently represents hydrogen, and a C₁-C₆alkyl or C₃-C₆cycloalkyl group;
R²⁴and R²⁵ each independently represents hydrogen, and a C₁-C₆alkyl or C₃-C₆ cycloalkyl group; or
R²⁴ and R²⁵ together with the nitrogen atom to which they are attached form a 3- to 8-membered saturated or partially saturated heterocyclic ring, optionally containing further heteroatoms or heterogroups selected from nitrogen, S(O)ₘ or oxygen;
R⁹, R¹², R¹⁵ and R²³ represent C₁-C₆alkyl or C₃-C₆ cycloalkyl;
R¹³ and R¹⁴ are defined as for R⁶ and R⁷ respectively;
R²⁰ represents a C₁-C₆alkyl optionally substituted by one or more substituents independently selected from halogen, hydroxyl or OR²³;
m, p, q and r each independently represent an integer 0, 1 or 2; and
A represents a C₆-C₁₀ aryl or C₅-C₁₂ heteroaryl group. See WO2008004948A1, U.S. Pat. Nos. 8,138,172, and 8,575,180, the disclosures of which are incorporated by reference in their entirties.

In some embodiments, the TLR7 and/or TLR8 agonist having the structure of:

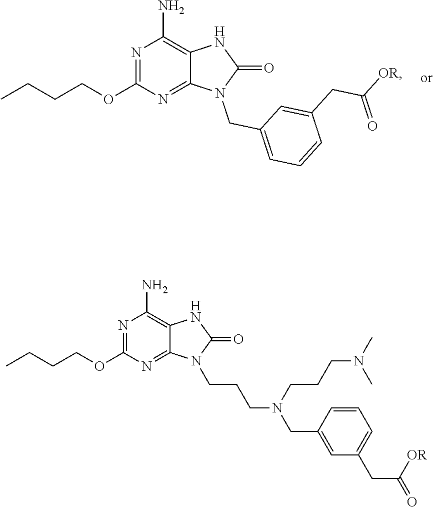

wherein R is Me or H.

In some embodiments, the TLR7 and/or TLR8 agonist having the structure of:

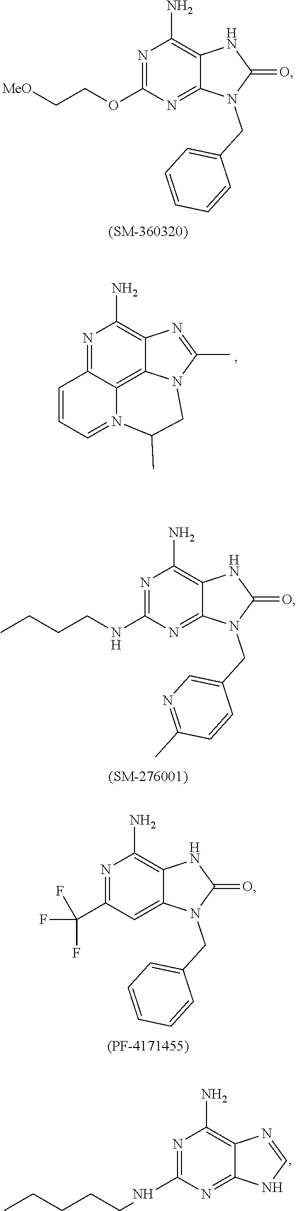

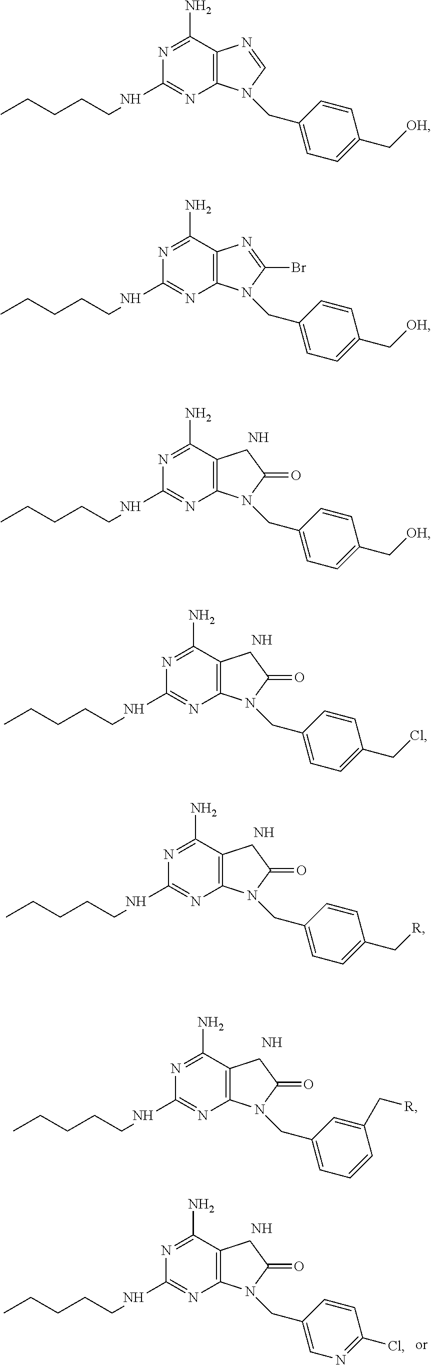

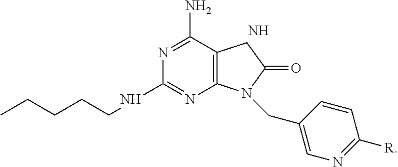

In some embodiments, the TLR7 and/or TLR8 agonist having the structure of Formula (XVI):

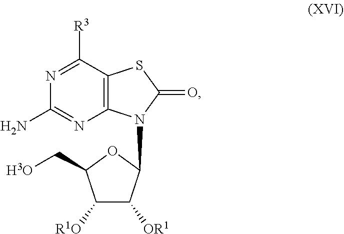

wherein: R¹ is independently H, —C(O)R³, or a racemic, L-, or D-amino acid group
—C(O)CHNH₂R⁴, wherein R³ is a substituted or unsubstituted alkyl, and R⁴ is H, or a substituted or unsubstituted alkyl;
R² is H, O, OR⁵, or N(R⁶)₂, wherein R⁵ is independently H or alkyl, and wherein R⁶ is independently H, substituted or unsubstituted alkyl, cycloalkyl, or together with nitrogen forms a substituted or unsubstituted heterocycloalkyl ring; and wherein if R is -OH, at least one of the R groups is a racemic, L-, or D-amino acid group —C(O)CHNH₂R⁴. See U.S. Pat. No. 6,924,271, the disclosure of which is incorporated by reference in its entirety.

In some embodiments, at least one of the R¹ groups is a racemic, L-, or D-amino acid group —C(O)CHNH₂R⁴, wherein R⁴ is a substituted or unsubstituted alkyl, and wherein the remaining R¹ groups are H; R² is OR⁵ or N(R⁶)₂, wherein R⁵is independently selected from H or alkyl, and wherein R is independently H, substituted or unsubstituted alkyl, cycloalkyl, or together with nitrogen forms a substituted or unsubstituted heterocycloalkyl ring.

In some embodiments, at least one of the R¹ groups is a L-amino acid group —C(O)CHNH₂R⁴, wherein R⁴ is a substituted or unsubstituted alkyl, and wherein the remaining R¹ groups are H; R² is OR⁵ or N(R⁶)₂, wherein R⁴ is a substituted alkyl, and wherein R⁶ is independently H or substituted or unsubstituted alkyl.

In some embodiments, at least one of the R¹ groups is a L-amino acid group —C(O)CHNH₂R , wherein R⁴ is —CH(CH₃)₂, and wherein the remaining R¹ groups are H; and R² is OH.

In some embodiments, the TLR7 and/or agonist is selected from the group consisting of:

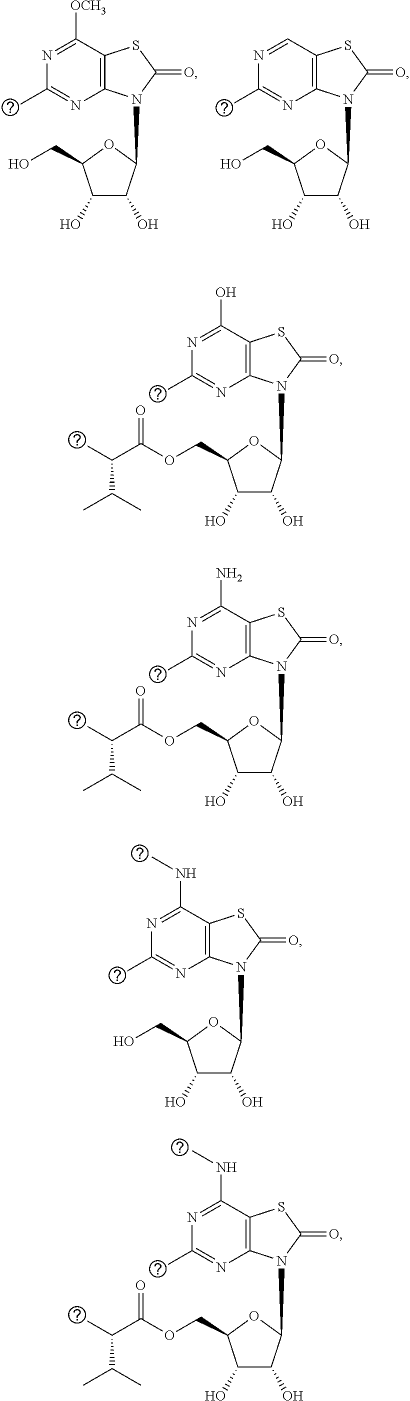 [SMILES: COc1nc(C)nc2c1sc(=O)n2[C@@H]1O[C@H](CO)[C@@H](O)[C@H]1O ; Cc1ncc2sc(=O)n([C@@H]3O[C@H](CO)[C@@H](O)[C@H]3O)c2n1 ; Cc1nc(O)c2sc(=O)n([C@@H]3O[C@H](COC(=O)[C@@H](C)C(C)C)[C@@H](O)[C@H]3O)c2n1 ; Cc1nc(N)c2sc(=O)n([C@@H]3O[C@H](COC(=O)[C@@H](C)C(C)C)[C@@H](O)[C@H]3O)c2n1 ; CNc1nc(C)nc2c1sc(=O)n2[C@@H]1O[C@H](CO)[C@@H](O)[C@H]1O ; CNc1nc(C)nc2c1sc(=O)n2[C@@H]1O[C@H](COC(=O)[C@@H](C)C(C)C)[C@@H](O)[C@H]1O]

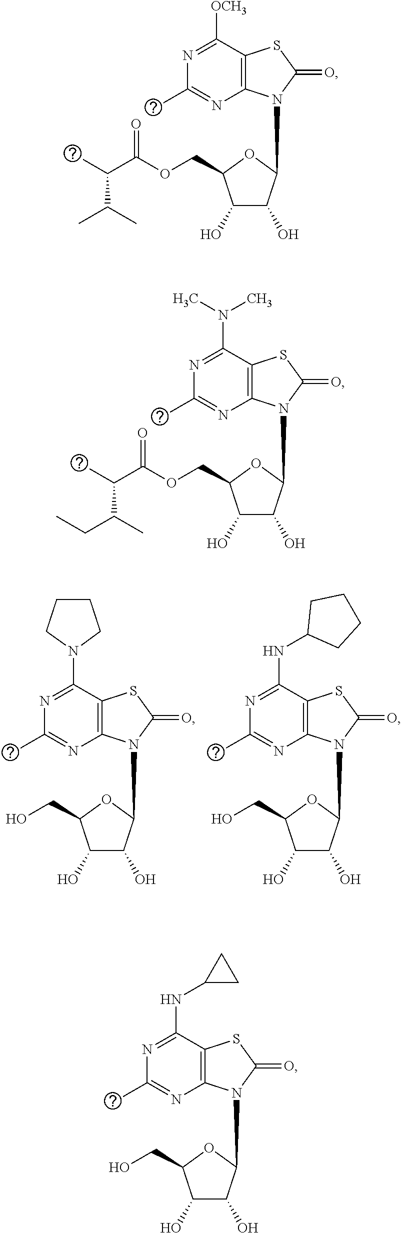 [SMILES: COc1nc(C)nc2c1sc(=O)n2[C@@H]1O[C@H](COC(=O)[C@@H](C)C(C)C)[C@@H](O)[C@H]1O ; CCC(C)[C@H](C)C(=O)OC[C@H]1O[C@@H](n2c(=O)sc3c(N(C)C)nc(C)nc32)[C@H](O)[C@@H]1O ; Cc1nc(N2CCCC2)c2sc(=O)n([C@@H]3O[C@H](CO)[C@@H](O)[C@H]3O)c2n1 ; Cc1nc(NC2CCCC2)c2sc(=O)n([C@@H]3O[C@H](CO)[C@@H](O)[C@H]3O)c2n1 ; Cc1nc(NC2CC2)c2sc(=O)n([C@@H]3O[C@H](CO)[C@@H](O)[C@H]3O)c2n1]

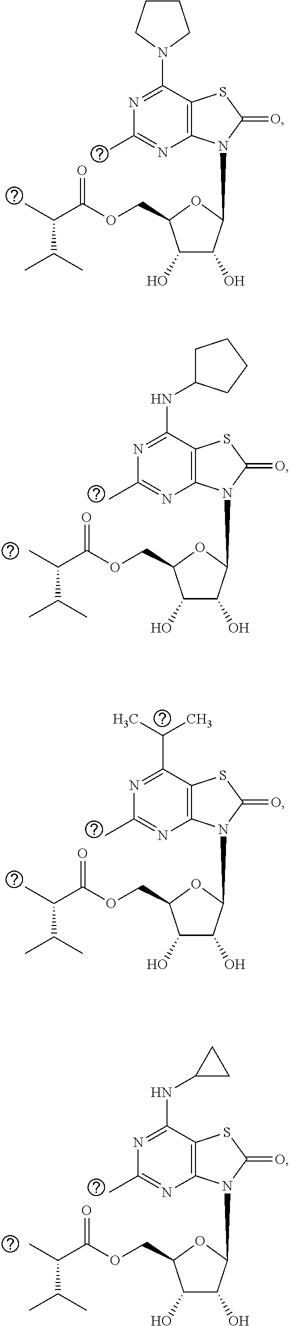 [SMILES: Cc1nc(N2CCCC2)c2sc(=O)n([C@@H]3O[C@H](COC(=O)[C@@H](C)C(C)C)[C@@H](O)[C@H]3O)c2n1 ; Cc1nc(NC2CCCC2)c2sc(=O)n([C@@H]3O[C@H](COC(=O)[C@@H](C)C(C)C)[C@@H](O)[C@H]3O)c2n1 ; Cc1nc(C(C)C)c2sc(=O)n([C@@H]3O[C@H](COC(=O)[C@@H](C)C(C)C)[C@@H](O)[C@H]3O)c2n1 ; Cc1nc(NC2CC2)c2sc(=O)n([C@@H]3O[C@H](COC(=O)[C@@H](C)C(C)C)[C@@H](O)[C@H]3O)c2n1]

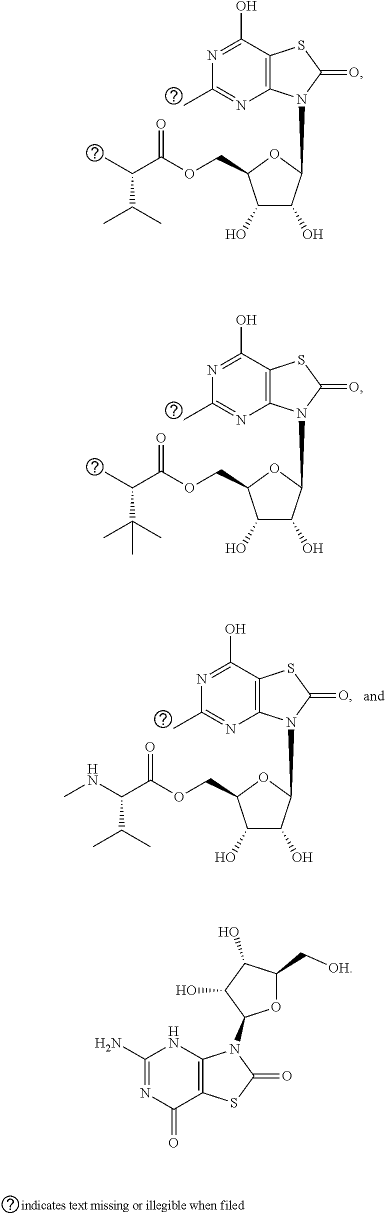 [SMILES: Cc1nc(O)c2sc(=O)n([C@@H]3O[C@H](COC(=O)[C@@H](C)C(C)C)[C@@H](O)[C@H]3O)c2n1 ; Cc1nc(O)c2sc(=O)n([C@@H]3O[C@H](COC(=O)[C@@H](C)C(C)(C)C)[C@@H](O)[C@H]3O)c2n1 ; CN[C@H](C(=O)OC[C@H]1O[C@@H](n2c(=O)sc3c(O)nc(C)nc32)[C@H](O)[C@@H]1O)C(C)C ; "and" ; Nc1nc(=O)c2sc(=O)n([C@@H]3O[C@H](CO)[C@@H](O)[C@H]3O)c2[nH]1]

In some embodiments, the TLR7 and/or TLR8 agonist having the structure of:

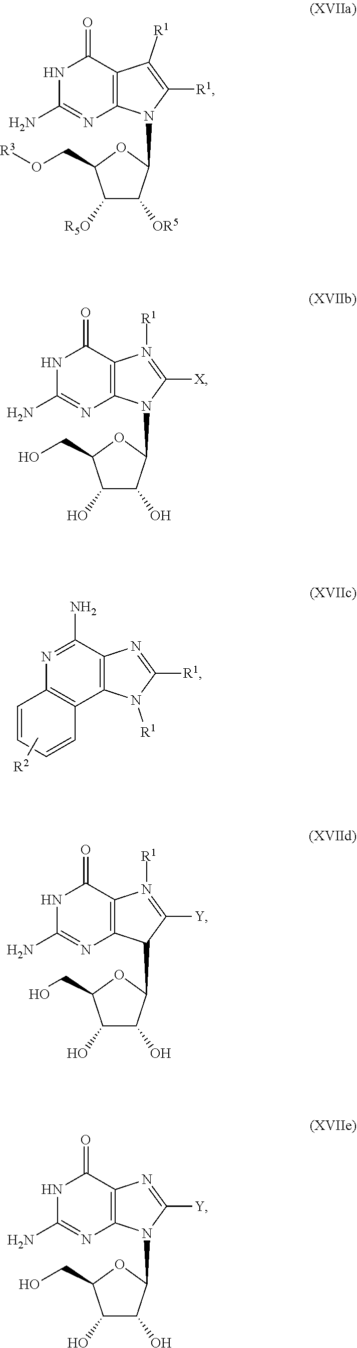

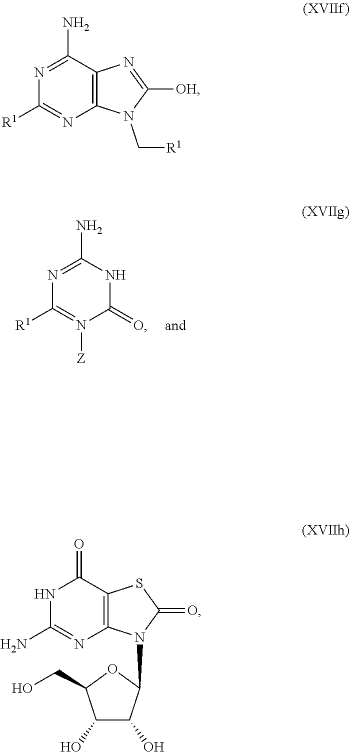

wherein:

each R¹ is H, or a substituted or unsubstituted alkyl, alkenyl, or alkynyl, which may be interrupted by one or more O, S, or N heteroatoms, or a substituted or unsubstituted aryl or heteroaryl;

R² is H, OH, SH, halo, or a substituted or unsubstituted alkyl, alkenyl, or alkynyl, which may be interrupted by one or more O, S, or N heteroatoms, or a substituted or unsubstituted —O-(alkyl), —O-(aryl), —O-(heteroaryl), —S-(alkyl), —S-(aryl), S-(heteroaryl), aryl, or heteroaryl;

R³ is H, OH, or SH, or a substituted or unsubstituted alkyl, alkenyl, alkynyl, aryl, heteroaryl, (alkyl), —O-(aryl), —O-(heteroaryl), —S-(alkyl), —S-(aryl), —S-(heteroaryl), —NH(alkyl), —NH(aryl), —NH(heteroaryl), —NH(R⁴)(alkyl), —NH(R⁴)(aryl), or —NH(R⁴)(heteroaryl), wherein R⁴ is a substituted or unsubstituted alkyl;

X is O or S;

Y is H, halo, OH, OR⁴, SH, SR⁴, or a substituted or unsubstituted alkyl or aryl; and

Z is H, halo, OH, OR⁴, SH, or SR⁴. See U.S. Pat. No. 7,576,068, the disclosure of which is incorporated by reference in its entirety.

In some embodiments, the TLR7 and/or TLR8 agonist having the structure of Formula (XVIII):

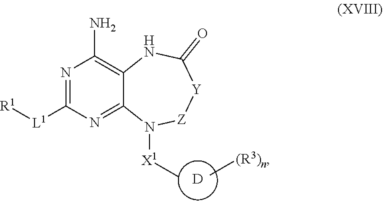

wherein:
Y—Z is CR⁴R⁵, —CR⁴R⁵—CR⁴R⁵—, —C(O)CR⁴R⁵—, —CR⁴R⁵C(O)—, —NR⁸C(O)—, —C(O)NR⁸—, —CR⁴R⁵S(O)₂—or —CR⁵═CR⁵—;
L¹ is —NR⁸—, —O—, —S—, —N(R⁸)C(O)—, —S(O)₂—, —S(O)—C(O)N(R⁸)—, —N(R⁸)S(O)₂—, —S(O)₂N(R⁸)— or a covalent bond;
R¹ is alkyl, substituted alkyl, haloalkyl, alkenyl, substituted alkenyl, alkynyl., substituted alkynyl, heteroalkyl, substituted heteroalkyl, carbocyclyl, substituted carbocyclyl, carbocyclylalkyl, substituted carbocyclylalkyl, heterocyclyl, substituted heterocyclyl, heterocyclylalkyl, or substituted heterocyclylalkyl, arylalkyl, substituted arylalkyl, heteroarylalkyl, substituted heteroarylalkyl, carbocyclylheteroalkyl, substituted carbocyclylheteroalkyl, heterocyclylheteroalkyl, substituted heterocyclylheteroalkyl, arylheteroalkyl, substituted arylheteroalkyl, heteroarylheteroalkyl, or substituted heteroarytheteroalkyl;

X¹ is alkylene, substituted alkylene, heteroalkylene, substituted heteroalkylene, alkenylene, substituted alkenylene, alkynylene, substituted alkynylene, carbocyclylene, substituted carbocyclylene, heteroclyclylene, substituted heterocyclylene, —NR⁸—, —O—, —C(O)—, —S(O)—, —S(O)₂—, or a bond;

D is carbocyclyl, substituted carbocyclyl, heterocyclyl or substituted heterocyclyl wherein said carboeyclyl, substituted carbocyclyl, heterocyclyl or substituted heterocyclyl is substituted with one or two -L²-NR⁶R⁷; or

D is a heterocyclyl, substituted heterocyclyl, heteroaryl or substituted heteroaryl wherein said heterocyclyl, substituted heterocyclyl, heteroaryl or substituted heteroaryl comprises one to four nitrogen atoms;

each L² is independently alkylene, substituted alkylene, heteroalkylene, substituted heteroalkylene, or a covalent bond: each R³ is independently halogen, cyano, azido, nitro, alkyl, substituted alkyl, hydroxyl, amino, heteroalkyl, substituted heteroalkyl, alkoxy, haloalkyl, haloalkoxy, —CHO, —C(O)OR⁸, —S(O)R⁸, —S(O)₂R⁸; —C(O)NR⁹R¹⁶, N(R⁹)C(O)R⁸, carbocyclyl, substituted carbocyclyl, carbocyclylakyl, substituted carbocyclylalkyl, alkenyl, substituted alkenyl, alkynyl, substituted alkynyl, S(O)₂NR⁹R¹⁰, —N(R⁹)S(O)₂R⁸, —N(R⁹)S(O)₂OR¹⁰, —OS(O)₂NR⁹R¹⁰;
n is 0, 1, 2, 3, 4 or 5;
R⁴ and R⁵ are each independently H, alkyl, substituted alkyl, haloalkyl, heteroalkyl, substituted heteroalkyl, carbocyclyl, substituted carbocyclyl, carbocyclylalkyl, substituted carbocyclylalkyl, heterocyclyl, substituted heterocyclyl, heterocyclylalkyl, substituted heterocyclylalkyl, arylalkyl, substituted arylalkyl, heteroarylalkyl, substituted heteroarylalkyl, carbocyclylheteroalkyl, substituted carbocyclylheteroalkyl, heterocyclylheteroalkyl, substituted heterocyclylheteroalkyl, arylheteroalkyl, substituted arylheteroalkyl, heteroarylheteroalkyl, or substituted heteroarylheteroalkyl, cyano, azido, OR⁸, —C(O)H, —C(O)R⁸, —S(O)R⁸, —S(O)₂R⁸, —C(O)OR⁸, or —C(O)NR⁹R¹⁰; or
R⁴ and R⁵, taken together with the carbon to which they are both attached, form a carbocycle, substituted carbocycle, heterocycle or substituted heterocycle; or
R⁴ and R⁵, when on the same carbon atom, taken together with the carbon to which they are attached are —C(O)— or —C(NR⁸)—; or
two R⁴ or two R⁵ on adjacent carbon atoms when taken together with the carbons to which they are attached form a 3 to 6 membered carbocycle, substituted carbocycle, heterocycle or substituted heterocycle;

R⁶ and R⁷ are each independently H, alkyl, substituted alkyl, alkenyl, substituted alkenyl, alkynyl, substituted alkynyl, haloalkyl, heteroalkyl, substituted heteroalkyl, carbocyclyl, substituted carbocyclyl, carbocyclylalkyl, substituted carbocyclylalkyl, heterocyclyl, substituted heterocyclyl, heterocyclylalkyl, substituted heterocyclylalkyl, arylalkyl, substituted arylalkyl, heteroarylalkyl, substituted heteroaralkyl, carbocyclylheteroalkyl, substituted carbocyclylheteroalkyl, heterocyclylheteroalkyl, substituted heterocyclylheteroalkyl, arylheteroalkyl, substituted arylheteroalkyl, heteroarytheteroalkyl, or substituted heteroarylheteroalkyl, —C(O)H, —C(O)R⁸, —S(O)R⁸, —S(O)₂R⁸, —C(O)OR⁸, or C(O)NR⁹R¹⁰, S(O)₂NR⁹R¹⁰; or
R⁶ and R⁷, taken together with the nitrogen to which they are both attached, form a substituted or unsubstituted heterocycle, which may contain one or more additional heteroatoms selected from N, O, P, or S; or
R⁷ taken together with L², and the N to which they are both attached, forms a substituted or unsubstituted 3 to 8 membered heterocycle which may contain one or more additional heteroatoms selected from N, O, S, or P;
R⁸ is H, alkyl, substituted alkyl, haloalkyl, alkenyl, substituted alkenyl, alkynyl, substituted alkynyl, heteroalkyl, substituted heteroalkyl, carbocyclyl, substituted carbocyclyl, carbocyclylalkyl, substituted carbocyclylalkyl, heterocyclyl, substituted heterocyclyl, heterocyclylalkyl, substituted heterocyclylalkyl, arylalkyl, substituted arylalkyl, heteroarylalkyl,substituted heteroarylalkyl, carbocyclylheteroalkyl, substituted carbocyclytheteroalkyl, heterocyclylheteroalkyl, substituted heterocyclylheteroalkyl, arylheteroalkyl, substituted arylheteroalkyl, heteroarylheteroalkyl, or substituted heteroarylheteroalkyl; and
R⁹ and R¹⁰ are each independently H, alkyl, substituted alkyl, alkenyl, substituted alkenyl, alkynyl, substituted alkynyl, haloalkyl, heteroalkyl, substituted heteroalkyl, carbocyclyl, substituted carbocyclyl, carbocyclylalkyl, substituted carbocyclylalkyl, heterocyclyl, substituted heterocyclyl, heterocyclylalkyl, substituted heterocyclylalkyl, arylalkyl, substituted arylalkyl, heteroarylalkyl, substituted heteroarylalkyl, carbocyclylheteroalkyl, substituted carbocyclylheteroalkyl, heterocyclylheteroalkyl, substituted heterocyclylheteroalkyl, arylheteroalkyl, substituted arylheteroalkyl, heteroarylheteroalkyl, or substituted heteroarylheteroalkyl; or
R⁹ and R¹⁰, taken together with the nitrogen to which they are both bonded, form a substituted or unsubstituted heterocycle,
wherein each substituted alkyl, substituted alkenyl, substituted alkynyl, substituted heteroalkyl, substituted carbocyclyl, substituted carbocyclylalkyl, substituted heterocyclyl, substituted heterocyclylalkyl, substituted arylalkyl, substituted heteroarylalkyl, substituted carbocyclylheteroalkyl, substituted heterocyclylheteroalkyl, substituted arylheteroalkyl, substituted heteroarylheteroalkyl, substituted alkylene, substituted heteroalkylene, substituted alkenylene, substituted alkynylene, substituted carbocyclylene, or substituted heterocyclylene is independently substituted with one to four substituents selected from the group consisting of -halogen, —R, —O, ═O, —OR, —SR, —S, —NR₂, —N(+)R₃, ═NR, —C(halogen)₃, —CR(halogen)₂, 13 CR₂(halogen), —CN, —OCN, —SCN, —N═C═O, —NCS, —NO, —NO₂, ═N₂, —N₃, —NRC(═O)R, —NRC(═O)OR, —NRC(═O)NRR, —C(═O)NRR, —C(═O)OR, —OC(═O)NRR, —OC(═O)OR, —C(═O)R, —S(═O)₂OR, —S(═O)₂R, —OS(═O)₂OR, —S(═O)₂NR, —S(═O)R, —NRS(═O)₂R, —NRS(═O)₂NRR, —NRS(═O)₂OR, —OP(═O)(OR)₂, —P(═O)(OR)₂, —P(O)(OR)(O)R, —C(═O)R, —C(═S)R, —C(═O)OR, —C(═S)OR, —C(═O)SR, —C(═S)SR, —C(═O)NRR, —C(═S)NRR, —C(═NR)NRR, and —NRC(═NR)NRR; wherein each R is independently H, alkyl, cycloalkyl, aryl, arylalkyl, or heterocyclyl. See US 20100143301 A1, the disclosure of which is incorporated by reference in its entirety.

In some embodiments, the TLR7 and/or TLR8 agonist having the structure of:

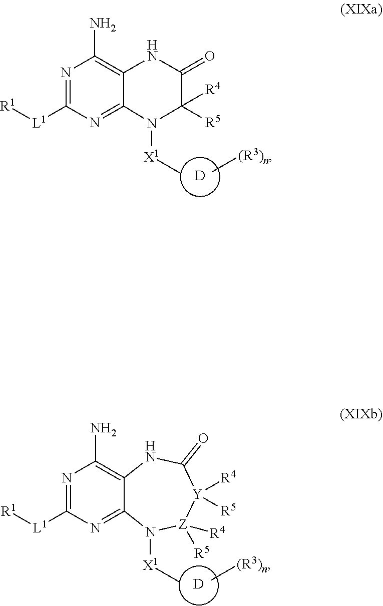

wherein:
is NH or O;
R¹ is alkyl, substituted alkyl, heteroalkyl, substituted heteroalkyl, heterocyclylalkyl, substituted heterocyclylalkyl, carbocyclylalkyl or substituted carbocyclylalkyl;
each of R⁴ and R⁵ independently is H or C₁-C₆alkyl or R⁴ and R⁵taken together with the carbon to which they are attached is —C(O)—;
X¹ is C₁-C₆alkylene, C₁-C₆ heteroalkylene or C₁-C₆ substituted heteroalkylene;
D is phenyl, biphenyl or pyridinyl, wherein said phenyl, biphenyl or pyridirtyl is substituted with -L²-NR⁶R⁷; or
D is pyridinyl, piperidinyl, piperazinyl or 1,2,3,4-tetrahydroisoquinolinyl n is 0 or 1;
R³ is halogen, cyano, alkyl, carbocyclyl, carbocyclylalkyl, haloalkyl, —C(O)OR⁶, —C(O)NR⁹R¹⁰ or —CHO;
L² is C₁-C₆alkylene or a covalent bond;
each of R⁶ and R⁷ independently is H, alkyl, or heteroaryl; or
R⁶ and R⁷ taken together with the nitrogen to which they are attached form a substituted or unsubstituted 4-6 membered heterocycle comprising 0 to 2 heteroatoms selected from N, O or S.
In some embodiments, the TLR7 and/or TLR8 agonist having the structure of:

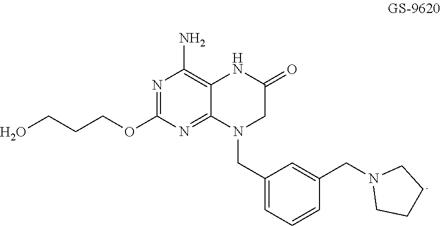 [SMILES: Nc1nc(OCCCO)nc2c1NC(=O)CN2Cc1cccc(CN2CCCC2)c1]

### C. Amount of Immunotherapeutics in the Therapeutic Combinations

In another aspect, the presently disclosed embodiments provide a therapeutic combination comprising a chemotherapeutic and an immunotherapeutic in an amount that is suitable for the combination therapy treatment of diseases such as tumors and cancers.

In some embodiments, the immunotherapeutic is of an amount that is capable of: (1) inducing IFN-α in an enriched human blood DCs; (2) inducing TNF-α in an enriched human blood DCs; and/or (3) inducing IL-12-α in an enriched human blood DCs.

Methods for measuring the activity of the immunotherapeutics include: 1) an assay to measure cytokines released from human dendritic cells stimulated by the immunotherapeutic; and 2) an efficacy study in a tumor model treated by the immunotherapeutic.

In some embodiments, the immunotherapeutic (e.g. resiquimod or its analogues) is administered, either orally or intravenously using oral formulation or intravenous formulation, respectively, in an amount so that the local concentration of the immunotherapeutic (e.g. near or at the tumor site of a solid tumor) is from about 0.005 μg/ml to about 1, 2, 3, 4, 5, 6, 7, 8, 9, 10, 11, or 12 μg/ml (all inclusive).

The local concentration of the immunotherapeutic (e.g. near or at the tumor site of a solid tumor) can measured using methods known in the art, such as measuring the tissue or serum concentration. Local effective concentration of therapeutic agent depends on its absorption from various routes, tissue distribution, and metabolism process, and plasma pharmacokinetics of the agent and tissue concentration could be measured routinely using methods known in the art.

In some embodiments, the immunotherapeutic is administered in an amount so that the local concentration of the immunotherapeutic (e.g., near or at the tumor site of a solid tumor) is from about 0.05 μg/ml, 0.1 μg/ml, 0.15 μg/ml, 0.2 μg/ml, 0.3 μg/ml, or 0.4 μg/ml, to about 0.5 μg/ml (all inclusive).

In some embodiments, the subject is administer an oral formulation comprising the immunotherapeutic (e.g. resiquimod or its analogues) in a dose of from about 0.001 mg/kg to about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, or 0.015 mg/kg, to about 0.02 mg/kg (all inclusive), two times per week. In some embodiments, the subject is administer an oral formulation comprising the immunotherapeutic (e.g. resiquimod or its analogues) in a dose of from about 0.0001mg/kg to about 0.0005 mg/kg, to about 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.015 mg/kg, or 0.02 mg/kg (all inclusive), two times per week.

In some embodiments, the subject is administer an oral formulation comprising the immunotherapeutic (e.g. resiquimod or its analogues) in a dose of from about 0.0001mg/kg to about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg , 0.008 mg/kg, 0.009 mg/kg, or 0.01 mg/kg, two times per week.

In some embodiments, the subject is administer an intravenous formulation comprising the immunotherapeutic (e.g. resiquimod or its analogues) in a dose of from about 0.0005mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, or about 0.015 mg/kg, to about 0.02 mg/kg (inclusive), weekly. In some embodiments, the subject is administer an intravenous formulation comprising the immunotherapeutic (e.g. resiquimod or its analogues) in a dose of from about 0.0005 mg/kg, to about 0.0006 mg/kg, 0.0007 mg/kg, 0.0008mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.015 mg/kg, or 0.02 mg/kg (inclusive), weekly.

In some embodiments, the method comprises administering to said subject an intravenous formulation comprising said immunotherapeutic (e.g. resiquimod or its analogues) in a dose of between about from 0.0008 mg/kg to about 0.0133 mg/kg, weekly.

In some embodiments, the subject is administer an intravenous formulation comprising the immunotherapeutic (e.g. resiquimod or its analogues) in a dose of less than or about 0.003 mg/kg, 0.004 mg/kg, 0.005 mg/kg, or 0.006 mg/kg to about 0.01 mg/kg, weekly. For references regarding safe dosage of immunotherapeutics, see Jurk et al., Nature Immunology, Vol. 4, No. 6:499 (2002), and Pockros et al., J. Hepatology, 47:174-182 (2007), the disclosures of which are incorporated by reference in their entirety.

### D. Targeted Therapeutics

In some embodiments, the immunotherapeutic is administrated in combination with a targeted therapeutic, either in a same formulation, or separately.

By “targeted therapeutic” herein is meant a therapeutic agent that binds specifically or selectively to a target molecule, cell, particle, tissue or aggregate, which generally is referred to as a “target” or a “marker,” and these are discussed in further detail herein.

By “therapeutic agent” herein is meant an agent that has therapeutic effect, such as in ameliorating or treating cancers.

In some embodiments, the targeted therapeutic comprises an immunoglobulin, a protein, a peptide, a small molecule, a nanoparticle, or a nucleic acid. In other embodiments one or more of these are specifically excluded.

In some embodiments, the targeted therapeutic comprises an antibody drug conjugate (ADC), as provided herein.

In some embodiments, the targeting therapeutic comprises an immunoglobulin, a protein, or a peptide, but does not contain a small molecule.

In some embodiments, the targeted therapeutic is not an ADC.

Exemplary targeted therapeutic such as antibodies (e.g., chimeric, humanized, and human) are recognized in the art and are useful without limitation in practicing the presently disclosed embodiments.

In some embodiments, a targeted therapeutic agent is an antibody, antibody fragment, bispecific antibody or other antibody-based molecule or compound.

In some embodiments, a targeted therapeutic agent is an aptamers, avimers, receptor-binding ligands, nucleic acids, biotin-avidin binding pairs, binding peptides or proteins, etc. that both binds specifically or preferably to a target molecule and has therapeutic effect such as against cancers or tumors.

By “target” or “marker” herein is meant any entity, such as Her2/Neu, that is capable of specifically binding to a particular targeted therapeutic. In some embodiments, targets are specifically associated with one or more particular cell or tissue types. In some embodiments, targets are specifically associated with one or more particular disease states. In some embodiments, targets are specifically associated with one or more particular developmental stages. For example, a cell type specific marker is typically expressed at levels at least 2 fold greater in that cell type than in a reference population of cells. In some embodiments, the cell type specific marker is present at levels at least 3 fold, at least 4 fold, at least 5 fold, at least 6 fold, at least 7 fold, at least 8 fold, at least 9 fold, at least 10 fold, at least 50 fold, at least 100 fold, or at least 1,000 fold greater than its average expression in a reference population. Detection or measurement of a cell type specific marker may make it possible to distinguish the cell type or types of interest from cells of many, most, or all other types. In some embodiments, a target can comprise a protein, a carbohydrate, a lipid, and/or a nucleic acid, as described herein.

By “specifically binds” or “preferably binds” herein is meant that the binding between two binding partners (e.g., between a targeting moiety and its binding partner) is selective for the two binding partners and can be discriminated from unwanted or non-specific interactions. For example, the ability of an antigen-binding moiety to bind to a specific antigenic determinant can be measured either through an enzyme-linked immunosorbent assay (ELISA) or other techniques familiar to one of skill in the art, e.g. surface plasmon resonance technique (analyzed on a BIAcore instrument) (Liljeblad et al., Glyco J 17, 323-329 (2000), and traditional binding assays (Heeley, Endocr Res 28, 217-229 (2002). The terms “anti-[antigen] antibody” and “an antibody that binds to [antigen]” refer to an antibody that is capable of binding the respective antigen with sufficient affinity such that the antibody is useful as a diagnostic and/or therapeutic agent in targeting the antigen. In some embodiments, the extent of binding of an anti-[antigen] antibody to an unrelated protein is less than about 10% of the binding of the antibody to the antigen as measured, e.g., by a radioimmunoassay (RIA). In some embodiments, an antibody that binds to antigen has a dissociation constant (KD) of <1 μM, <100 nM, <10 nM, <1 nM, <0.1 nM, <0.01 nM, or <0.001 nM (e.g., 10⁻⁸ M or less, from 10⁻⁸ M to 10⁻¹³ M, or from 10⁻⁹ M to 10⁻¹³ M). It is understood that the above definition is also applicable to non-antibody antigen-binding moieties that bind to an antigen.

In certain specific embodiments, a target is a tumor marker. In some embodiments, a tumor marker is an antigen that is present in a tumor that is not present in normal organs, tissues, and/or cells. In some embodiments, a tumor marker is an antigen that is more prevalent in a tumor than in normal organs, tissues, and/or cells. In some embodiments, a tumor marker is an antigen that is more prevalent in malignant cancer cells than in normal cells.

By “tumor antigen” herein is meant an antigenic substance produced in tumor cells that can also trigger an immune response in the host. Normal proteins in the body are not immunogenic because of self-tolerance, a process in which self-reacting cytotoxic T lymphocytes (CTLs) and autoantibody-producing B lymphocytes are culled “centrally” in primary lymphatic tissue (BM) and “peripherally” in secondary lymphatic tissue (mostly thymus for T-cells and spleen/lymph nodes for B cells). Thus any protein that is not exposed to the immune system during its maturation may trigger an immune response. This may include normal proteins that are well sequestered from the immune system, proteins that are normally produced in extremely small quantities, proteins that are normally produced only in certain stages of development, or proteins whose structure is modified due to mutation.

In some embodiments, a target is preferentially expressed in tumor tissues and/or cells versus normal tissues and/or cells.

In some embodiments a marker is a tumor marker. The marker may be a polypeptide that is expressed at higher levels by dividing than by non-dividing cells. For example, Her-2/neu (also known as ErbB-2) is a member of the EGF receptor family and is expressed on the cell surface of tumors associated with breast cancer. Another example is a peptide known as F3 that is a suitable targeting agent for directing a nanoparticle to nucleolin (Porkka et al., 2002, Proc. Natl. Acad. Sci., USA, 99:7444; and Christian et al., 2003, J. Cell Biol., 163:871). It has been shown that targeted particles comprising a nanoparticle and the Al0 aptamer (which specifically binds to PSMA) were able to specifically and effectively deliver docetaxel to prostate cancer tumors.

Antibodies or other drugs that specifically target these tumor targets can specifically interfere with and regulate signaling pathways of the biological behavior of tumor cells, regulate directly, or block signaling pathways, to inhibit tumor cell growth or induce apoptosis. To date, there are dozens of targeted drugs have been approved for solid tumors or hematological malignancies.

In some embodiments, the tumor antigen (or tumor target or tumor marker) is selected from the group consisting of: CD2, CD19, CD20, CD22, CD27, CD33, CD37, CD38, CD40, CD44, CD47, CD52, CD56, CD70, CD79, and CD137.

In some embodiments, the tumor antigen (or tumor target or tumor marker) is selected from the group consisting of: 4-1BB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin αν, Integrin ανβ, KIR, LAG-3, Lewis Y antigen, Mesothelin, c-MET, MN Carbonic anhydrase IX, MUC1, MUC16, Nectin-4, NKGD2, NOTCH, OX40, OX4OL, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, and variants thereof. The variants of these tumor antigens encompass various mutants or polymorphisms known in the art and/or that naturally occurred.

In some embodiments, the targeted therapeutic comprises an antibody, or a functional fragment thereof.

By immunoglobulin” or “antibody” herein is meant a full-length (e.g., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment. An antibody or antibody fragment may be conjugated or otherwise derivatized within the scope of the claimed subject matter. Such antibodies include IgG1, lgG2a, IgG3, IgG4 (and IgG4 subforms), as well as IgA isotypes.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity The terms “full-length antibody”, “intact antibody”, and “whole antibody” are used herein interchangeably to refer to an antibody having a structure substantially similar to a native antibody structure or having heavy chains that contain an Fc region. Antibody also encompasses immunoglobulins that have been engineered to possess additional domains, for example a whole antibody or antibody fragment fused with an additional variable domain, as might be done to create a bispecific antibody.

By “native antibodies” herein is meant naturally occurring immunoglobulin molecules (that is, immunoglobulin molecules possessing a structure that could arise from immunization of an animal) with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CHI, CH2, and CH3), also called a heavy chain constant region Similarly, from N- to C-terminus, each light chain has a variable region (VL), also called a variable light domain or a light chain variable domain, followed by a constant light (CL) domain, also called a light chain constant region. The light chain of an antibody may be assigned to one of two types, called kappa (κ) and lambda (λ), based on the amino acid sequence of its constant domain.

By “antibody fragment” herein is meant a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g. scFv), single-domain antibodies, and multispecific antibodies formed from antibody fragments. For a review of certain antibody fragments, see Hudson et al., Nat Med 9, 129-134 (2003). For a review of scFv fragments, see e.g. Pliickthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B 1). Antibody fragments can be made by various techniques, including but not limited to proteolytic digestion of an intact antibody as well as production by recombinant host cells (e.g. E. coli or phage), as described herein.

By “antigen binding domain” herein is meant the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable region (VL) and an antibody heavy chain variable region (VH).

By “variable region” or “variable domain” herein is meant the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

By “hypervariable region” or “HVR” herein is meant each of the regions of an antibody variable domain which are hypervariable in sequence and/or form structurally defined loops “hypervariable loops”). Generally, native four-chain antibodies comprise six HVRs; three in the VH (HI, H2, H3), and three in the VL (LI, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The antibody of the presently disclosed embodiments can be a chimeric antibody, a humanized antibody, a human antibody, or an antibody fusion protein.

By “chimeric antibody” herein is meant a recombinant protein that contains the variable domains of both the heavy and light antibody chains, including the complementarity determining regions (CDRs) of an antibody derived from one species, preferably a rodent antibody, more preferably a murine antibody, while the constant domains of the antibody molecule are derived from those of a human antibody. For veterinary applications, the constant domains of the chimeric antibody may be derived from that of other species, such as a subhuman primate, cat or dog.

By “humanized antibody” herein is meant a recombinant protein in which the CDRs from an antibody from one species; e.g., a rodent antibody, are transferred from the heavy and light variable chains of the rodent antibody into human heavy and light variable domains. The constant domains of the antibody molecule are derived from those of a human antibody. In some embodiments, specific residues of the framework region of the humanized antibody, particularly those that are touching or close to the CDR sequences, may be modified, for example replaced with the corresponding residues from the original rodent, subhuman primate, or other antibody.

By “human antibody” herein is meant an antibody obtained, for example, from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al, Nature Genet. 7: 13 (1994), Lonberg et al, Nature 368:856 (1994), and Taylor et al, Int. Immun 6:579 (1994). A fully human antibody also can be constructed by genetic or chromosomal transfection methods, as well as phage display technology, all of which are known in the art. See for example, McCafferty et al, Nature 348:552-553 (1990) for the production of human antibodies and fragments thereof in vitro, from immunoglobulin variable domain gene repertoires from unimmunized donors. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see e.g. Johnson and Chiswell, Current Opinion in Structural Biology 3:5564-571 (1993). Human antibodies may also be generated by in vitro activated B cells. See U.S. Pat. Nos. 5,567,610 and 5,229,275, which are incorporated herein by reference in their entirety.

By “antibody fusion protein” herein is meant a recombinantly-produced antigen-binding molecule in which two or more of the same or different natural antibody, single-chain antibody or antibody fragment segments with the same or different specificities are linked. A fusion protein comprises at least one specific binding site. Valency of the fusion protein indicates the total number of binding arms or sites the fusion protein has to antigen(s) or epitope(s); i.e., monovalent, bivalent, trivalent or multivalent. The multivalency of the antibody fusion protein means that it can take advantage of multiple interactions in binding to an antigen, thus increasing the avidity of binding to the antigen, or to different antigens. Specificity indicates how many different types of antigen or epitope an antibody fusion protein is able to bind; i.e., monospecific, bispecific, trispecific, multispecific. Using these definitions, a natural antibody, e.g., an IgG, is bivalent because it has two binding arms but is monospecific because it binds to one type of antigen or epitope. A monospecific, multivalent fusion protein has more than one binding site for the same antigen or epitope. For example, a monospecific diabody is a fusion protein with two binding sites reactive with the same antigen. The fusion protein may comprise a multivalent or multispecific combination of different antibody components or multiple copies of the same antibody component. The fusion protein may additionally comprise a therapeutic agent.

In some embodiments, the targeting moiety comprises a probody, such as those disclosed in U.S. Pat. Nos. 8,518,404; 8,513,390; and US Pat. Appl. Pub. Nos.; 20120237977A1, 20120149061A1, 20130150558A1, the disclosures of which are incorporated by reference in their entireties.

Probodies are monoclonal antibodies that are selectively activated within the cancer microenvironment, focusing the activity of therapeutic antibodies to tumors and sparing healthy tissue.

In general, the probody comprises at least an antibody or antibody fragment thereof (collectively referred to as “AB”), capable of specifically binding a target, wherein the AB is modified by a masking moiety (MM). When the AB is modified with a MM and is in the presence of the target, specific binding of the AB to its target is reduced or inhibited, as compared to the specific binding of the AB not modified with an MM or the specific binding of the parental AB to the target. The dissociation constant (Kd) of the MM towards the AB is generally greater than the Kd of the AB towards the target. When the AB is modified with a MM and is in the presence of the target, specific binding of the AB to its target can be reduced or inhibited, as compared to the specific binding of the AB not modified with an MM or the specific binding of the parental AB to the target. When an AB is coupled to or modified by a MM, the MM can ‘mask’ or reduce, or inhibit the specific binding of the AB to its target. When an AB is coupled to or modified by a MM, such coupling or modification can effect a structural change which reduces or inhibits the ability of the AB to specifically bind its target.

In some embodiments, the probody is an activatable antibodies (AAs) where the AB modified by an MM can further include one or more cleavable moieties (CM). Such AAs exhibit activatable/switchable binding, to the AB's target. AAs generally include an antibody or antibody fragment (AB), modified by or coupled to a masking moiety (MM) and a modifiable or cleavable moiety (CM). In some embodiments, the CM contains an amino acid sequence that serves as a substrate for a protease of interest. In other embodiments, the CM provides a cysteine-cysteine disulfide bond that is cleavable by reduction. In yet other embodiments the CM provides a photolytic substrate that is activatable by photolysis.

The CM and AB of the AA may be selected so that the AB represents a binding moiety for a target of interest, and the CM represents a substrate for a protease that is co-localized with the target at a treatment site in a subject. Alternatively or in addition, the CM is a cysteine-cysteine disulfide bond that is cleavable as a result of reduction of this disulfide bond. AAs contain at least one of a protease-cleavable CM or a cysteine-cysteine disulfide bond, and in some embodiments include both kinds of CMs. The AAs can alternatively or further include a photolabile substrate, activatable by a light source. The AAs disclosed herein find particular use where, for example, a protease capable of cleaving a site in the CM is present at relatively higher levels in target-containing tissue of a treatment site (for example diseased tissue; for example for therapeutic treatment or diagnostic treatment) than in tissue of non-treatment sites (for example in healthy tissue). The AAs disclosed herein also find particular use where, for example, a reducing agent capable of reducing a site in the CM is present at relatively higher levels in target-containing tissue of a treatment or diagnostic site than in tissue of non-treatment non-diagnostic sites. The AAs disclosed herein also find particular use where, for example, a light source, for example, by way of laser, capable of photolysing a site in the CM is introduced to a target-containing tissue of a treatment or diagnostic site.

In some embodiments, AAs can provide for reduced toxicity and/or adverse side effects that could otherwise result from binding of the AB at non-treatment sites if the AB were not masked or otherwise inhibited from binding its target. Where the AA contains a CM that is cleavable by a reducing agent that facilitates reduction of a disulfide bond, the ABs of such AAs may selected to exploit activation of an AB where a target of interest is present at a desired treatment site characterized by elevated levels of a reducing agent, such that the environment is of a higher reduction potential than, for example, an environment of a non-treatment site.

In general, an AA can be designed by selecting an AB of interest and constructing the remainder of the AA so that, when conformationally constrained, the MM provides for masking of the AB or reduction of binding of the AB to its target. Structural design criteria to be taken into account to provide for this functional feature.

In some embodiments, the targeted therapeutic is an antibody, or antibody fragment, that is selected based on its specificity for an antigen expressed on a target cell, or at a target site, of interest. A wide variety of tumor-specific or other disease-specific antigens have been identified and antibodies to those antigens have been used or proposed for use in the treatment of such tumors or other diseases. The antibodies that are known in the art can be used as a targeted therapeutic in a therapeutic combination, in particular for the treatment of the disease with which the target antigen is associated. Examples of target antigens (and their associated diseases) to which a target therapeutic can be targeted include: CD2, CD19, CD20, CD22, CD27, CD33, CD37, CD38, CD40, CD44, CD47, CD52, CD56, CD70, CD79, CD137, 4-1BB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin αν, Integrin ανβ, KIR, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2,VEGFR-1, VEGFR-2, VEGFR-3.

In some embodiments, the antibody is selected from the group consisting of: Rituxan (rituximab), Herceptin (trastuzumab), Erbitux (cetuximab), Vectibix (Panitumumab), Arzerra (Ofatumumab), Benlysta (belimumab), Yervoy (ipilimumab), Perjeta (Pertuzumab), Tremelimumab, Opdivo (Nivolumab, ONO-4538, BMS-936558, or MDX1106), Dacetuzumab, Urelumab, MPDL3280A, Lambrolizumab, and Blinatumomab, CT-011, Keytruda (pembrolizumab, MK-3475), BMS-936559, MPDL3280A, MED14736, or MSB0010718C.

Rituxan (Rituximab) is a chimeric antibody used for the treatment of B-cell non-Hodgkin's lymphoma. It acts on the surface of B cells expressing the CD20 antigen that is expressed on 90% of B-cell non-Hodgkin's lymphoma. Rituxan binds CD20 to induce B cell lysis through CDC and ADCC, as well as sensitize human lymphocytes that are drug resistance for some cytotoxic chemotherapeutics.

Herceptin (Trastuzumab) is a humanized monoclonal antibody that acts on human epidermal growth factor receptor extracellular domain of Her2, which is expressed in 25% -30% of breast cancer. It is believed that Trastuzumab has anti-tumor effect through (1) down-regulation Her2 receptor, inhibition of Her2 intracellular signaling transduction pathways and induction of apoptosis; (2) immune mechanisms related antibody dependent ADCC and CDC to kill tumor cells; (3) enhance the effects of chemotherapy.

Erbitux (Cetuximab) is a chimeric antibody that acts on epidermal growth factor receptor (EGFR). Erbitux binds EGFR to inhibit its signal transduction pathway, affecting cell proliferation, invasion and metastasis, and angiogenesis. Inhibition of EGFR signal transduction pathway can enhance chemotherapy drugs and radiation therapy efficacy.

Avastin (Bevacizumab) is a humanized monoclonal antibody that targets vascular endothelial growth factor (VEGF). Its binding of VEGFR inhibits VEGF and signal transduction, resulting in inhibition of tumor angiogenesis.

Other antibodies that currently under development can also be used as targeted therapeutic. For example, therapeutic monoclonal antibodies against the following targets are under development for treatment of tumors: CD2, CD19, CD20, CD22, CD27, CD33, CD37, CD38, CD40, CD44, CD47, CD52, CD56, CD70, CD79, and CD137 and the following targets for treatment of tumors: 4-1BB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin αν, Integrin ανβ, KIR, LAG-3, Lewis, Mesothelin, c-MET, MN Carbonic anhydrase IX, MUC1, MUC16, Nectin-4, NKGD2, NOTCH, OX40, OX4OL, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, and VEGFR-3 and their variant. (Scott A M, Wolchok J D, Old L J, Antibody Therapy of Cancer, Nat Rev Cancer 2012 Mar. 22, 12(4):278-87).

In some embodiments, the targeted therapeutic comprises a Fab, Fab′, F(ab′)2, single domain antibody, T and Abs dimer, Fv, scFv, dsFv, ds-scFv, Fd, linear antibody, minibody, diabody, bispecific antibody fragment, bibody, tribody, sc-diabody, kappa (lamda) body, BiTE, DVD-Ig, SIP, SMIP, DART, or an antibody analogue comprising one or more CDRs.

Table 1 shows various antibody structures and the targets being studied.

TABLE 1

| Antibody Structure | Exemplary Target |
|---|---|
| scFv | CC49, ERBB2, Ley |
| Diabody | Ley and TAG-72 |
| Affibody | ERBB2 |
| Minibody | CEA, ERBB2 |
| Protein-Fc | Angiopoietin 1, angiopoietin 2, VEGFR1, |
| | VEGFR2 |
| Intact IgG | CD20, CD33, EGFR, ERBB2, VEGF |
| IgE and IgM | GM2 |
| Drug conjugates | CD30, CD33 and ERBB2 |
| Loaded nanoparticles | A33, EGFR and transferrin |
| Bispecifics | CD19-CD3, EPCAM-CD3, gp100-CD3 |

### Targeted Therapeutics Comprising a Targeting Moiety

In some aspects, the targeted therapeutic of the presently disclosed embodiments presently disclosed embodiments comprise a targeting moiety, such as an ADC.

By “targeting moiety (TM)” or “targeting agent” here in is meant a molecule, complex, or aggregate, that binds specifically or selectively to a target molecule, cell, particle, tissue or aggregate, which generally is referred to as a “target” or a “marker,” and these are discussed in further detail herein.

In some embodiments, the targeting moiety comprises an immunoglobulin, a protein, a peptide, a small molecule, a nanoparticle, or a nucleic acid.

Exemplary targeting agents such as antibodies (e.g., chimeric, humanized and human), ligands for receptors, lectins, and saccharides, and substrate for certain enzymes are recognized in the art and are useful without limitation in practicing the presently disclosed embodiments. Other targeting agents include a class of compounds that do not include specific molecular recognition motifs include nanoparticles, macromolecules such as poly(ethylene glycol), polysaccharide, and polyamino acids which add molecular mass to the activating moiety. The additional molecular mass affects the pharmacokinetics of the activating moiety, e.g., serum half-life.

In some embodiments, a targeting moiety is an antibody, antibody fragment, bispecific antibody or other antibody-based molecule or compound. However, other examples of targeting moieties are known in the art and may be used, such as aptamers, avimers, receptor-binding ligands, nucleic acids, biotin-avidin binding pairs, binding peptides or proteins, etc. The terms “targeting moiety” and “binding moiety” are used synonymously herein.

By “target” or “marker” herein is meant any entity that is capable of specifically binding to a particular targeting moiety. In some embodiments, targets are specifically associated with one or more particular cell or tissue types. In some embodiments, targets are specifically associated with one or more particular disease states. In some embodiments, targets are specifically associated with one or more particular developmental stages. For example, a cell type specific marker is typically expressed at levels at least 2 fold greater in that cell type than in a reference population of cells. In some embodiments, the cell type specific marker is present at levels at least 3 fold, at least 4 fold, at least 5 fold, at least 6 fold, at least 7 fold, at least 8 fold, at least 9 fold, at least 10 fold, at least 50 fold, at least 100 fold, or at least 1,000 fold greater than its average expression in a reference population. Detection or measurement of a cell type specific marker may make it possible to distinguish the cell type or types of interest from cells of many, most, or all other types. In some embodiments, a target can comprise a protein, a carbohydrate, a lipid, and/or a nucleic acid, as described herein.

A substance is considered to be “targeted” for the purposes described herein if it specifically binds to a nucleic acid targeting moiety. In some embodiments, a nucleic acid targeting moiety specifically binds to a target under stringent conditions. An inventive complex or compound comprising targeting moiety is considered to be “targeted” if the targeting moiety specifically binds to a target, thereby delivering the entire complex or compound composition to a specific organ, tissue, cell, extracellular matrix component, and/or intracellular compartment.

In certain embodiments, compound in accordance with the presently disclosed embodiments comprise a targeting moiety which specifically binds to one or more targets (e.g. antigens) associated with an organ, tissue, cell, extracellular matrix component, and/or intracellular compartment. In some embodiments, compounds comprise a targeting moiety which specifically binds to targets associated with a particular organ or organ system. In some embodiments, compounds in accordance with the presently disclosed embodiments comprise a nuclei targeting moiety which specifically binds to one or more intracellular targets (e.g. organelle, intracellular protein). In some embodiments, compounds comprise a targeting moiety which specifically binds to targets associated with diseased organs, tissues, cells, extracellular matrix components, and/or intracellular compartments. In some embodiments, compounds comprise a targeting moiety which specifically binds to targets associated with particular cell types (e.g. endothelial cells, cancer cells, malignant cells, prostate cancer cells, etc.).

In some embodiments, compounds in accordance with the presently disclosed embodiments presently disclosed embodiments comprise a targeting moiety which binds to a target that is specific for one or more particular tissue types (e.g. liver tissue vs. prostate tissue). In some embodiments, compounds in accordance with the presently disclosed embodiments presently disclosed embodiments comprise a targeting moiety which binds to a target that is specific for one or more particular cell types (e.g. T cells vs. B cells). In some embodiments, compounds in accordance with the presently disclosed embodiments presently disclosed embodiments comprise a targeting moiety which binds to a target that is specific for one or more particular disease states (e.g. tumor cells vs. healthy cells). In some embodiments, compounds in accordance with the presently disclosed embodiments presently disclosed embodiments comprise a targeting moiety which binds to a target that is specific for one or more particular developmental stages (e.g. stem cells vs. differentiated cells).

In some embodiments, a target may be a marker that is exclusively or primarily associated with one or a few cell types, with one or a few diseases, and/or with one or a few developmental stages. A cell type specific marker is typically expressed at levels at least 2 fold greater in that cell type than in a reference population of cells which may consist, for example, of a mixture containing cells from a plurality (e.g., 5-10 or more) of different tissues or organs in approximately equal amounts. In some embodiments, the cell type specific marker is present at levels at least 3 fold, at least 4 fold, at least 5 fold, at least 6 fold, at least 7 fold, at least 8 fold, at least 9 fold, at least 10 fold, at least 50 fold, at least 100 fold, or at least 1000 fold greater than its average expression in a reference population. Detection or measurement of a cell type specific marker may make it possible to distinguish the cell type or types of interest from cells of many, most, or all other types.

In some embodiments, a target comprises a protein, a carbohydrate, a lipid, and/or a nucleic acid. In some embodiments, a target comprises a protein and/or characteristic portion thereof, such as a tumor-marker, integrin, cell surface receptor, transmembrane protein, intercellular protein, ion channel, membrane transporter protein, enzyme, antibody, chimeric protein, glycoprotein, etc. In some embodiments, a target comprises a carbohydrate and/or characteristic portion thereof, such as a glycoprotein, sugar (e.g., monosaccharide, disaccharide, polysaccharide), glycocalyx (i.e., the carbohydrate-rich peripheral zone on the outside surface of most eukaryotic cells) etc. In some embodiments, a target comprises a lipid and/or characteristic portion thereof, such as an oil, fatty acid, glyceride, hormone, steroid (e.g., cholesterol, bile acid), vitamin (e.g. vitamin E), phospholipid, sphingolipid, lipoprotein, etc. In some embodiments, a target comprises a nucleic acid and/or characteristic portion thereof, such as a DNA nucleic acid; RNA nucleic acid; modified DNA nucleic acid; modified RNA nucleic acid; nucleic acid that includes any combination of DNA, RNA, modified DNA, and modified RNA.

Numerous markers are known in the art. Typical markers include cell surface proteins, e.g., receptors. Exemplary receptors include, but are not limited to, the transferrin receptor; LDL receptor; growth factor receptors such as epidermal growth factor receptor family members (e.g., EGFR, Her2, Her3, Her4) or vascular endothelial growth factor receptors, cytokine receptors, cell adhesion molecules, integrins, selectins, and CD molecules. The marker can be a molecule that is present exclusively or in higher amounts on a malignant cell, e.g., a tumor antigen.

In some embodiments, the targeting moiety binds to a tumor cell specifically or preferably in comparison to a non-tumor cell.

The binding of target moiety to tumor cell can be measured using assays known in the art.

In some embodiments, the tumor cell is of a carcinoma, a sarcoma, a lymphoma, a myeloma, or a central nervous system cancer.

In some embodiments, the targeting moiety is capable of binding to a tumor antigen specifically or preferably in comparison to a non-tumor antigen.

In certain specific embodiments, a target is a tumor marker. In some embodiments, a tumor marker is an antigen that is present in a tumor that is not present in normal organs, tissues, and/or cells. In some embodiments, a tumor marker is an antigen that is more prevalent in a tumor than in normal organs, tissues, and/or cells. In some embodiments, a tumor marker is an antigen that is more prevalent in malignant cancer cells than in normal cells.

In some embodiments, the targeting moiety comprises folic acid or a derivative thereof.

In recent years, research on folic acid had made great progress. Folic acid is a small molecule vitamin that is necessary for cell division. Tumor cells divide abnormally and there is a high expression of folate receptor (FR) on tumor cell surface to capture enough folic acid to support cell division.

Data indicate FR expression in tumor cells is 20-200 times higher than normal cells. The expression rate of FR in various malignant tumors are: 82% in ovarian cancer, 66% in non-small cell lung cancer, 64% in kidney cancer, 34% in colon cancer, and 29% in breast cancer (Xia W, Low P S. Late-targeted therapies for cancer, J Med Chem. 2010; 53(19):6811-24). The expression rate of FA and the degree of malignancy of epithelial tumor invasion and metastasis is positively correlated. FA enters cell through FR mediated endocytosis, and FA through its carboxyl group forms FA complexes with drugs which enter the cells. Under acidic conditions (pH value of 5), FR separates from the FA, and FA releases drugs into the cytoplasm.

Clinically, the system can be used to deliver drugs selectively attack the tumor cells. Folic acid has small molecular weight, has non-immunogenicity and high stability, and is inexpensive to synthesis. More importantly, chemical coupling between the drug and the carrier is simple, and as such using FA as targeting molecule to construct drug delivery system has become a research hotspot for cancer treatment. Currently EC145 (FA chemotherapy drug conjugate compound) that is in clinical trials can effectively attack cancer cells (Pribble P and Edelman M J. EC145: a novel targeted agent for adenocarcinoma of the lung, Expert Opin. Investig. Drugs, 2012, 21:755-761).

In some embodiments, the targeting moiety comprises extracellular domains (ECD) or soluble form of PD-1, PDL-1, CTLA4, CD47, BTLA, KIR, TIM3, 4-1BB, and LAG3, full length of partial of a surface ligand Amphiregulin, Betacellulin, EGF, Ephrin, Epigen, Epiregulin, IGF, Neuregulin, TGF, TRAIL, or VEGF. In some embodiments these extracellular domains or soluble forms may be fused to an antibody Fc domain (sometimes called an immunoadhesin).

In some embodiments, the targeting moiety comprises a Fab, Fab′, F(ab′)2, single domain antibody, T and Abs dimer, Fv, scFv, dsFv, ds-scFv, Fd, linear antibody, minibody, diabody, bispecific antibody fragment, bibody, tribody, sc-diabody, kappa (lamda) body, BiTE, DVD-Ig, SIP, SMIP, DART, or an antibody analogue comprising one or more CDRs.

In some embodiments, the targeting moiety is an antibody, or antibody fragment, that is selected based on its specificity for an antigen expressed on a target cell, or at a target site, of interest. A wide variety of tumor-specific or other disease-specific antigens have been identified and antibodies to those antigens have been used or proposed for use in the treatment of such tumors or other diseases. The antibodies that are known in the art can be used as a targeted therapeutic in the herein disclosed combinations, in particular for the treatment of the disease with which the target antigen is associated. Examples of target antigens (and their associated diseases) include: CD2, CD19, CD20, CD22, CD27, CD33, CD37, CD38, CD40, CD44, CD47, CD52, CD56, CD70, CD79, CD137, 4-1BB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin αν, Integrin ανβ, KIR, LAG-3, Lewis Y, Mesothelin, c-MET, MN Carbonic anhydrase IX, MUC1, MUC16, Nectin-4, NKGD2, NOTCH, OX40, OX4OL, PD-1, PDL1, PSCA, PSMA, RANKL, ROR1, ROR2, SLC44A4, Syndecan-1, TACI, TAG-72, Tenascin, TIM3, TRAILR1, TRAILR2,VEGFR-1, VEGFR-2, VEGFR-3. In some embodiments the targeted therapeutic can comprise an antibody-linker-drug conjugate. In other embodiments antibody-linker-drug conjugates are specifically excluded.

In some embodiments, the targeting moiety comprises an nucleic acid targeting moiety.

In general, a nucleic acid targeting moiety is any polynucleotide that binds to a component associated with an organ, tissue, cell, extracellular matrix component, and/or intracellular compartment (the target).

In some embodiments, the nucleic acid targeting moieties are aptamers.

An aptamer is typically a polynucleotide that binds to a specific target structure that is associated with a particular organ, tissue, cell, extracellular matrix component, and/or intracellular compartment. In general, the targeting function of the aptamer is based on the three-dimensional structure of the aptamer. In some embodiments, binding of an aptamer to a target is typically mediated by the interaction between the two- and/or three-dimensional structures of both the aptamer and the target. In some embodiments, binding of an aptamer to a target is not solely based on the primary sequence of the aptamer, but depends on the three-dimensional structure(s) of the aptamer and/or target. In some embodiments, aptamers bind to their targets via complementary Watson-Crick base pairing which is interrupted by structures (e.g. hairpin loops) that disrupt base pairing.

In some embodiments, the nucleic acid targeting moieties are spiegelmers (PCT Publications WO 98/08856, WO 02/100442, and WO 06/117217). In general, spiegelmers are synthetic, mirror-image nucleic acids that can specifically bind to a target (i.e. mirror image aptamers). Spiegelmers are characterized by structural features which make them not susceptible to exo- and endo-nucleases.

One of ordinary skill in the art will recognize that any nucleic acid targeting moiety (e.g. aptamer or spiegelmer) that is capable of specifically binding to a target can be used in accordance with the presently disclosed embodiments. In some embodiments, nucleic acid targeting moieties to be used in accordance with the presently disclosed embodiments may target a marker associated with a disease, disorder, and/or condition. In some embodiments, nucleic acid targeting moieties to be used in accordance with the presently disclosed embodiments may target cancer-associated targets. In some embodiments, nucleic acid targeting moieties to be used in accordance with the presently disclosed embodiments may target tumor markers. Any type of cancer and/or any tumor marker may be targeted using nucleic acid targeting moieties in accordance with the presently disclosed embodiments. To give but a few examples, nucleic acid targeting moieties may target markers associated with prostate cancer, lung cancer, breast cancer, colorectal cancer, bladder cancer, pancreatic cancer, endometrial cancer, ovarian cancer, bone cancer, esophageal cancer, liver cancer, stomach cancer, brain tumors, cutaneous melanoma, and/or leukemia.

Nucleic acids of the presently disclosed embodiments (including nucleic acid nucleic acid targeting moieties and/or functional RNAs to be delivered, e.g., RNAi-inducing entities, ribozymes, tRNAs, etc., described in further detail below) may be prepared according to any available technique including, but not limited to chemical synthesis, enzymatic synthesis, enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in molecular biology, v. 288 (Clifton, N.J.) Totowa, N.J.: Humana Press, 2005).

The nucleic acid that forms the nucleic acid targeting moiety may comprise naturally occurring nucleosides, modified nucleosides, naturally occurring nucleosides with hydrocarbon linkers (e.g., an alkylene) or a polyether linker (e.g., a PEG linker) inserted between one or more nucleosides, modified nucleosides with hydrocarbon or PEG linkers inserted between one or more nucleosides, or a combination of thereof. In some embodiments, nucleotides or modified nucleotides of the nucleic acid nucleic acid targeting moiety can be replaced with a hydrocarbon linker or a polyether linker provided that the binding affinity and selectivity of the nucleic acid nucleic acid targeting moiety is not substantially reduced by the substitution (e.g., the dissociation constant of the nucleic acid nucleic acid targeting moiety for the target should not be greater than about 1×10⁻³ M).

It will be appreciated by those of ordinary skill in the art that nucleic acids in accordance with the presently disclosed embodiments may comprise nucleotides entirely of the types found in naturally occurring nucleic acids, or may instead include one or more nucleotide analogs or have a structure that otherwise differs from that of a naturally occurring nucleic acid. U.S. Pat. Nos. 6,403,779; 6,399,754; 6,225,460; 6,127,533; 6,031,086; 6,005,087; 5,977,089; and references therein disclose a wide variety of specific nucleotide analogs and modifications that may be used. See Crooke, S. (ed.) Antisense Drug Technology: Principles, Strategies, and Applications, Marcel Dekker; ISBN: 0824705661; 1st edition (2001) and references therein. For example, 2′-modifications include halo, alkoxy and allyloxy groups. In some embodiments, the T-OH group is replaced by a group selected from H, OR, R, halo, SH, SR, NH2, NHR, NR2 or CN, wherein R is C1-C6alkyl, alkenyl, or alkynyl, and halo is F, Cl, Br, or I. Examples of modified linkages include phosphorothioate and 5′-N-phosphoramidite linkages.

Nucleic acids comprising a variety of different nucleotide analogs, modified backbones, or non-naturally occurring internucleoside linkages can be utilized in accordance with the presently disclosed embodiments. Nucleic acids of the presently disclosed embodiments may include natural nucleosides (i.e., adenosine, thymidine, guanosine, cytidine, uridine, deoxy adenosine, deoxythymidine, deoxyguanosine, and deoxycytidine) or modified nucleosides. Examples of modified nucleotides include base modified nucleosides (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadeosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, O(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display improved properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Modified nucleic acids need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially affected. To give but one example, modifications may be located at any position of a nucleic acid targeting moiety such that the ability of the nucleic acid targeting moiety to specifically bind to the target is not substantially affected. The modified region may be at the 5′-end and/or the 3′-end of one or both strands. For example, modified nucleic acid targeting moieties in which approximately 1-5 residues at the 5′ and/or 3′ end of either of both strands are nucleotide analogs and/or have a backbone modification have been employed. The modification may be a 5′ or 3′ terminal modification. One or both nucleic acid strands may comprise at least 50% unmodified nucleotides, at least 80% unmodified nucleotides, at least 90% unmodified nucleotides, or 100% unmodified nucleotides.

Nucleic acids in accordance with the presently disclosed embodiments may, for example, comprise a modification to a sugar, nucleoside, or internucleoside linkage such as those described in U.S. Patent Application Publications 2003/0175950, 2004/0192626, 2004/0092470, 2005/0020525, and 2005/0032733. The presently disclosed embodiments encompass the use of any nucleic acid having any one or more of the modification described therein. For example, a number of terminal conjugates, e.g., lipids such as cholesterol, lithocholic acid, aluric acid, or long alkyl branched chains have been reported to improve cellular uptake. Analogs and modifications may be tested using, e.g., using any appropriate assay known in the art, for example, to select those that result in improved delivery of a therapeutic or diagnostic agent, improved specific binding of an nucleic acid targeting moiety to a target, etc. In some embodiments, nucleic acids in accordance with the presently disclosed embodiments may comprise one or more non-natural nucleoside linkages. In some embodiments, one or more internal nucleotides at the 3′-end, 5′-end, or both 3′- and 5′-ends of the nucleic acid targeting moiety are inverted to yield a linkage such as a 3′-3′ linkage or a 5′-5′ linkage.

In some embodiments, nucleic acids in accordance with the presently disclosed embodiments are not synthetic, but are naturally-occurring entities that have been isolated from their natural environments.

Any method can be used to design novel nucleic acid targeting moieties (see, e.g., U.S. Pat. Nos. 6,716,583; 6,465,189; 6,482,594; 6,458,543; 6,458,539; 6,376,190; 6,344,318; 6,242,246; 6,184,364; 6,001,577; 5,958,691; 5,874,218; 5,853,984; 5,843,732; 5,843,653; 5,817,785; 5,789,163; 5,763,177; 5,696,249; 5,660,985; 5,595,877; 5,567,588; and 5,270,163; and U.S. Patent Application Publications 2005/0069910, 2004/0072234, 2004/0043923, 2003/0087301, 2003/0054360, and 2002/0064780). The presently disclosed embodiments provide methods for designing novel nucleic acid targeting moieties. The presently disclosed embodiments further provides methods for isolating or identifying novel nucleic acid targeting moieties from a mixture of candidate nucleic acid targeting moieties.

Nucleic acid targeting moieties that bind to a protein, a carbohydrate, a lipid, and/or a nucleic acid can be designed and/or identified. In some embodiments, nucleic acid targeting moieties can be designed and/or identified for use in targeted therapeutics to enable binding to proteins and/or characteristic portions thereof, such as tumor-markers, integrins, cell surface receptors, transmembrane proteins, intercellular proteins, ion channels, membrane transporter proteins, enzymes, antibodies, chimeric proteins etc. In some embodiments, nucleic acid targeting moieties can be designed and/or identified for use in targeted therapeutics to enable binding to carbohydrates and/or characteristic portions thereof, such as glycoproteins, sugars (e.g., monosaccharides, disaccharides and polysaccharides), glycocalyx (i.e., the carbohydrate-rich peripheral zone on the outside surface of most eukaryotic cells) etc. In some embodiments, nucleic acid targeting moieties can be designed and/or identified for use in targeted therapeutics to enable binding to lipids and/or characteristic portions thereof, such as oils, saturated fatty acids, unsaturated fatty acids, glycerides, hormones, steroids (e.g., cholesterol, bile acids), vitamins (e.g. vitamin E), phospholipids, sphingolipids, lipoproteins etc. In some embodiments, nucleic acid targeting moieties can be designed and/or identified for use in targeted therapeutics to enable binding nucleic acids and/or characteristic portions thereof, such as DNA nucleic acids; RNA nucleic acids; modified DNA nucleic acids; modified RNA nucleic acids; and nucleic acids that include any combination of DNA, RNA, modified DNA, and modified RNA; etc.

Nucleic acid targeting moieties (e.g. aptamers or spiegelmers) may be designed and/or identified using any available method. In some embodiments, nucleic acid targeting moieties are designed and/or identified by identifying nucleic acid targeting moieties from a candidate mixture of nucleic acids. Systemic Evolution of Ligands by Exponential Enrichment (SELEX), or a variation thereof, is a commonly used method of identifying nucleic acid targeting moieties that bind to a target from a candidate mixture of nucleic acids.

Nucleic acid targeting moieties that bind selectively to any target can be isolated by the SELEX process, or a variation thereof, provided that the target can be used as a target in the SELEX process.

### III. Pharmaceutical Formulations and Administration

The presently disclosed embodiments further relate to a pharmaceutical formulation comprising a compound of the combination (that is, a chemotherapeutic, an immunotherapeutic, or a targeted therapeutic) or a pharmaceutically acceptable salt thereof, and one or more pharmaceutically acceptable carriers.

The pharmaceutical formulations including pharmaceutically acceptable carriers described herein can be delivered to a patient using a wide variety of routes or modes of administration. Suitable routes of administration include, inhalation, transdermal, oral, rectal, transmucosal, intestinal and parenteral administration, including intramuscular, subcutaneous and intravenous injections. Preferably, the compounds of the combination comprising an immune modulator (that is, an immunotherapeutic) are administered parenterally, more preferably intravenously.

As used herein, the terms “administering” or “administration” are intended to encompass all means for directly and indirectly delivering a compound to its intended site of action.

The compounds described herein, or pharmaceutically acceptable salts and/or hydrates thereof, may be administered singly, in combination with other compounds of the combination, and/or in cocktails combined with other therapeutic agents. Of course, the choice of therapeutic agents that can be co-administered with the compounds of the combination will depend, in part, on the condition being treated. In other embodiments use of additional therapeutic agents to those of the herein disclosed combinations is specifically excluded.

For example, when administered to patients suffering from a disease state caused by an organism that relies on an autoinducer, the compounds of the combination can be administered in cocktails containing agents used to treat the pain, infection and other symptoms and side effects commonly associated with the disease. Such agents include, e.g., analgesics, antibiotics, etc.

When administered to a patient undergoing cancer treatment, the compounds may be administered in cocktails containing anti-cancer agents and/or supplementary potentiating agents. The compounds may also be administered in cocktails containing agents that treat the side-effects of radiation therapy, such as anti-emetics, radiation protectants, etc.

Supplementary potentiating agents that can be co-administered with the compounds of the invention include, e.g., tricyclic anti-depressant drugs (e.g., imipramine, desipramine, amitriptyline, clomipramine, trimipramine, doxepin, nortriptyline, protriptyline, amoxapine and maprotiline); non-tricyclic and anti-depressant drugs (e.g., sertraline, trazodone and citalopram); Ca+2 antagonists (e.g., verapamil, nifedipine, nitrendipine and caroverine); amphotericin; triparanol analogues (e.g., tamoxifen); antiarrhythmic drugs (e.g., quinidine); antihypertensive drugs (e.g., reserpine); thiol depleters (e.g., buthionine and sulfoximine); and calcium leucovorin.

The active compound(s) of the combination are administered per se or in the form of a pharmaceutical composition wherein the active compound(s) is in admixture with one or more pharmaceutically acceptable carriers, excipients or diluents. Pharmaceutical compositions for use in accordance with the presently disclosed embodiments are typically formulated in a conventional manner using one or more physiologically acceptable carriers comprising excipients and auxiliaries, which facilitate processing of the active compounds into preparations which, can be used pharmaceutically. Proper formulation is dependent upon the route of administration chosen.

For transmucosal administration, penetrants appropriate to the barrier to be permeated are used in the formulation. Such penetrants are generally known in the art.

For oral administration, the compounds can be formulated readily by combining the active compound(s) with pharmaceutically acceptable carriers well known in the art. Such carriers enable the compounds of the combination to be formulated as tablets, pills, dragees, capsules, liquids, gels, syrups, slurries, and suspensions for oral ingestion by a patient to be treated. Pharmaceutical preparations for oral use can be obtained solid excipient, optionally grinding a resulting mixture, and processing the mixture of granules, after adding suitable auxiliaries, if desired to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner

For administration by inhalation, the compounds for use according to the presently disclosed embodiments are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

One or more of the components of the herein disclosed combinations may be formulated for parenteral administration by injection, e.g., by bolus injection or continuous infusion. Injection is a preferred method of administration. Formulations for injection may be presented in unit dosage form, e.g., in ampoules or in multi-dose containers, with an added preservative. The formulations may take such forms as suspensions, solutions or emulsions in oily or aqueous vehicles, and may contain formulatory agents such as suspending, stabilizing and/or dispersing agents may be added, such as the cross-linked polyvinyl pyrrolidone, agar, or alginic acid or a salt thereof such as sodium alginate.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions. For injection, the components of the herein disclosed combinations may be formulated in aqueous solutions, preferably in physiologically compatible buffers such as Hanks's solution, Ringer's solution, or physiological saline buffer.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation or transcutaneous delivery (e.g., subcutaneously or intramuscularly), intramuscular injection or a transdermal patch. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include calcium carbonate, calcium phosate, various sugars, starches, cellulose derivatives, gelatin, and polymers such as polyethylene glycols.

In some embodiments, the pharmaceutical composition of the presently disclosed embodiments further comprises an additional therapeutic agent.

In some embodiments, the additional therapeutic agent is an anticancer agent.

In some embodiments, the additional anticancer agent is selected from an antimetabolite, an inhibitor of topoisomerase I and II, an alkylating agent, a microtubule inhibitor, an antiandrogen agent, a GNRh modulator or mixtures thereof.

In some embodiments, the additional therapeutic agent is a chemotherapeutic agent.

By “chemotherapeutic agent” herein is meant a chemical compound useful in the treatment of cancer. Examples are but not limited to: Gemcitabine, Irinotecan, Doxorubicin, 5-Fluorouracil, Cytosine arabinoside (“Ara-C”), Cyclophosphamide, Thiotepa, Busulfan, Cytoxin, TAXOL, Methotrexate, Cisplatin, Melphalan, Vinblastine and Carboplatin.

In some embodiments, the second chemotherapeutic agent is selected from the group consisting of tamoxifen, raloxifene, anastrozole, exemestane, letrozole, imatanib, paclitaxel, cyclophosphamide, lovastatin, minosine, gemcitabine, cytarabine, 5-fluorouracil, methotrexate, docetaxel, goserelin, vincristine, vinblastine, nocodazole, teniposide etoposide, gemcitabine, epothilone, vinorelbine, camptothecin, daunorubicin, actinomycin D, mitoxantrone, acridine, doxorubicin, epirubicin, or idarubicin.

### IV. Kits

In another aspect, the presently disclosed embodiments provide kits containing the therapeutic combinations provided herein and directions for using the therapeutic combinations. The kit may also include a container and optionally one or more vial, test tube, flask, bottle, or syringe. Other formats for kits will be apparent to those of skill in the art and are within the scope of the presently disclosed embodiments.

### V. Medical Use

In another aspect, the presently disclosed embodiments provides a method for treating a disease condition in a subject that is in need of such treatment, comprising: administering to the subject a therapeutic combination or pharmaceutical composition comprising a therapeutically effective amount of the compound of the presently disclosed embodiments or a pharmaceutically acceptable salt thereof, and a pharmaceutical acceptable carrier.

In addition to the compositions and constructs described above, the presently disclosed embodiments also provide a number of uses of the therapeutic combinations. The presently disclosed combinations comprise a chemotherapeutic that has one or more of the functions of inhibitors of myeloid-derived suppressor cells. These uses comprise administering to an animal such as a mammal or a human in need thereof an effective amount of a compound of the presently disclosed embodiments.

The presently disclosed combination is useful for treating diseases such as cancer in a subject, such as a human being. Combinations and uses for treating tumors by providing a subject the compounds of the combination in a pharmaceutically acceptable composition and manner, with a pharmaceutically effective amount of a composition of the presently disclosed embodiments are provided.

By “cancer” herein is meant the pathological condition in humans that is characterized by unregulated cell proliferation. Examples include but are not limited to: carcinoma, sarcoma, lymphoma, blastoma, and leukemia. More particular examples of cancers include but are not limited to: Acute myeloid leukemia (AML), Breast cancer, Chronic lymphocytic leukemia (CLL), Chronic myelogenous leukemia (CML), Hodgkin lymphoma, Multiple myeloma, Mycosis fungoides, Neuroblastoma, Non-Hodgkin lymphoma (NHL), Ovarian cancer, and Retinoblastoma.

By “inhibiting” or “treating” or “treatment” herein is meant to reduction, therapeutic treatment and prophylactic or preventative treatment, wherein the objective is to reduce or prevent the aimed pathologic disorder or condition. In one example, following administering of a compound of the presently disclosed embodiments, a cancer patient may experience a reduction in tumor size. “Treatment” or “treating” includes (1) inhibiting a disease in a subject experiencing or displaying the pathology or symptoms of the disease, (2) ameliorating a disease in a subject that is experiencing or displaying the pathology or symptoms of the disease, and/or (3) affecting any measurable decrease in a disease in a subject or patient that is experiencing or displaying the pathology or symptoms of the disease. To the extent a compound of the presently disclosed embodiments may prevent growth and/or kill cancer cells, it may be cytostatic and/or cytotoxic.

The term “treating” or “treatment” broadly includes any kind of treatment activity, including the mitigation, or prevention of disease in man or other animals, or any activity that otherwise affects the structure or any function of the body of man or other animals Treatment activity includes the administration of the medicaments, dosage forms, and pharmaceutical compositions described herein to a patient, especially according to the various methods of treatment disclosed herein, whether by a healthcare professional, the patient him/herself, or any other person. Treatment activities include the orders, instructions, and advice of healthcare professionals such as physicians, physician's assistants, nurse practitioners, and the like, that are then acted upon by any other person including other healthcare professionals or the patient him/herself. With respect to the presently disclosed combinations, different components may be administered by different persons; however, this activity should be seen as being undertaken on the orders of the prescribing or overseeing healthcare professional, such as but not limited to an oncologist or hospitalist, or alternatively, at the behest of the patient, and for the purpose of patient obtaining benefit of the combination. In some embodiments, treatment activity can also include encouraging, inducing, or mandating that a particular medicament, or combination thereof, be chosen for treatment of a condition—and the medicament is actually used—by approving insurance coverage for the medicament, denying coverage for an alternative medicament, including the medicament or combination on, or excluding an alternative medicament or combination, from a drug formulary, or offering a financial incentive to use the medicament or combination, as might be done by an insurance company or a pharmacy benefits management company, and the like. In some embodiments, treatment activity can also include encouraging, inducing, or mandating that a particular medicament of combination be chosen for treatment of a condition—and the medicament or combination is actually used—by a policy or practice standard as might be established by a hospital, clinic, health maintenance organization, medical practice or physicians group, and the like. In addition to the therapeutic benefit to the patients, the treatments herein disclosed may be engaged in for benefits related to cost and cost-efficiency for all parties, and benefits related to overall improved outcomes in a population of patients accruing the healthcare professionals, institutions and insurers.

Cancer treatment is often conducted in cycles. As used herein a cycle of treatment at its most basic refers to a time interval during which drug is administered followed by a time interval when it is not administered. A new cycle begins when administration of the drug recommences. The interval in which the drug is not administered provides an opportunity for the body to recover from an adverse side effect of the treatment and/or for medical personnel to evaluate the effectiveness of the treatment. Additionally, when two active agents are administered in combination, as in presently disclosed embodiments, the two active agents may be given on different schedules, with the schedule of one of the agents defining a beginning of a cycle. In some embodiments an active agent may be administered a fixed number of times, with a fixed periodicity, and/or over a fixed time interval. In other embodiments any one or more of these parameters may be variable. In particular embodiments as described herein, a cycle begins with administration of the chemotherapeutic component followed, for example, 1-7 days later, by a first administration of the immunotherapeutic component. Initiation of administration of the immunotherapeutic may also be based on achieving a threshold degree of reduction of MDCS. In some embodiments the chemotherapeutic is administered a single time per cycle. In some embodiments administration of the chemotherapeutic is continued past the point at which administration of the immunotherapeutic commences. In some embodiments, administration of the chemotherapeutic ceases after a certain time interval. In some embodiment administration of the chemotherapeutic is repeated at certain time intervals within the cycle or when MDCS numbers rise to more than a threshold proportion of their pretreatment level, for example, >30, >40, >50, >60, >70, or >80% of their pretreatment level. In some embodiments administration of the immunotherapeutic continues for an interval within the cycle after administration of the chemotherapeutic ceases. In some embodiments a cycle is defined by the cessation and re-initiation of administration of the chemotherapeutic component. In alternative embodiments administration of both the chemotherapeutic and immunotherapeutic agents ceases for a period of time prior to a new cycle being initiated.

By “therapeutically effective amount” herein is meant an amount of a compound provided herein effective to “treat” a disorder in a subject or mammal In the case of cancer, the therapeutically effective amount of the drug may either reduce the number of cancer cells, reduce the tumor size, inhibit cancer cell infiltration into peripheral organs, inhibit tumor metastasis, inhibit tumor growth to certain extent, and/or relieve one or more of the symptoms associated with the cancer to some extent.

The effectiveness of cancer therapy is typically measured in terms of “response.” The techniques to monitor responses can be similar to the tests used to diagnose cancer such as, but not limited to:
A lump or tumor involving some lymph nodes can be felt and measured externally by physical examination.
Some internal cancer tumors will show up on an x-ray or CT scan and can be measured with a ruler.
Blood tests, including those that measure organ function can be performed.
A tumor marker test can be done for certain cancers.
Regardless of the test used, whether blood test, cell count, or tumor marker test, it is repeated at specific intervals so that the results can be compared to earlier tests of the same type.
Response to cancer treatment is defined several ways:
Complete response—all of the cancer or tumor disappears; there is no evidence of disease. Expression level of tumor marker (if applicable) may fall within the normal range.
Partial response—the cancer has shrunk by a percentage but disease remains. Levels of a tumor marker (if applicable) may have fallen (or increased, based on the tumor marker, as an indication of decreased tumor burden) but evidence of disease remains.
Stable disease—the cancer has neither grown nor shrunk; the amount of disease has not changed. A tumor marker (if applicable) has not changed significantly.
Disease progression—the cancer has grown; there is more disease now than before treatment. A tumor marker test (if applicable) shows that a tumor marker has risen.

Other measures of the efficacy of cancer treatment include intervals of overall survival (that is time to death from any cause, measured from diagnosis or from initiation of the treatment being evaluated)), cancer-free survival (that is, the length of time after a complete response cancer remains undetectable), and progression-free survival (that is, the length of time after disease stabilization or partial response that resumed tumor growth is not detectable).

There are two standard methods for the evaluation of solid cancer treatment response with regard to tumor size (tumor burden), the WHO and RECIST standards. These methods measure a solid tumor to compare a current tumor with past measurements or to compare changes with future measurements and to make changes in a treatment regimen. In the WHO method, the solid tumor's long and short axes are measured with the product of these two measurements is then calculated; if there are multiple solid tumors, the sum of all the products is calculated. In the RECIST method, only the long axis is measured. If there are multiple solid tumors, the sum of all the long axes measurements is calculated. However, with lymph nodes, the short axis is measured instead of the long axis.

Administration “in combination with” one or more further therapeutic agents includes simultaneous (concurrent) and consecutive administration in any order. In some embodiments “pharmaceutical composition” refers to a product obtained from mixing or combining active ingredients, and includes both fixed and non-fixed combinations of the active ingredients. The term “fixed combination” means that the active ingredients, are administered to a patient simultaneously in the form of a single entity or dosage. The term “non-fixed combination” means that the active ingredients are administered to a patient as separate entities either simultaneously, concurrently or sequentially with no specific time limits, wherein such administration provides therapeutically effective levels of the active ingredients in the body of the patient. The latter also applies to cocktail therapy, e.g. the administration of three or more active ingredients.

In some embodiments, the diseases condition is tumor or cancer. In some embodiments, the cancer or tumor is selected from stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer or lymphoma.

In some embodiments, the disease condition comprises abnormal cell proliferation, such as a pre-cancerous lesion.

The currently disclosed combinations are particularly useful for the treatment of cancer and for the inhibition of the multiplication of a tumor cell or cancer cell in an animal Cancer, or a precancerous condition, includes a tumor, metastasis, or any disease or disorder characterized by uncontrolled cell growth, can be treated or prevented by administration the chemotherapeutic-immunotherapeutic combination. In some embodiments comprising a targeted therapeutic, the targeting moiety specifically binds to or associates with a cancer-cell or a tumor-cell-associated antigen. In some embodiments comprising an immunotherapeutic and a targeted therapeutic the two can be conjugated, that is, they are provided as an antibody-drug conjugate (ADC). In some embodiments the ADC is internalized after binding to a cell, for example, by receptor-mediated endocytosis (TLR7 and TLR8 are endosomal receptors). The antigen can be attached to a tumor cell or cancer cell or can be an extracellular matrix protein associated with the tumor cell or cancer cell. Once inside the cell, the linker is hydrolytically or enzymatically cleaved by a tumor-cell or cancer-cell-associated protease, thereby releasing the immunotherapeutic. The released immunotherapeutic is then free to diffuse and induce or enhance immune activity of immune cells or tumor cells. In an alternative embodiment, the immunotherapeutic is cleaved in the tumor microenvironment, and the immunotherapeutic subsequently penetrates the cell.

Representative examples of precancerous conditions that may be targeted by the compounds of the presently disclosed embodiments, include: metaplasia, hyperplysia, dysplasia, colorectal polyps, actinic ketatosis, actinic cheilitis, human papillomaviruses, leukoplakia, lychen planus and Bowen's disease.

Representative examples of cancers or tumors that may be targeted by compounds of the presently disclosed embodiments include: lung cancer, colon cancer, prostate cancer, lymphoma, melanoma, breast cancer, ovarian cancer, testicular cancer, CNS cancer, renal cancer, kidney cancer, pancreatic cancer, stomach cancer, oral cancer, nasal cancer, cervical cancer and leukemia. It will be readily apparent to the ordinarily skilled artisan that the particular targeting moiety used in the compound can be chosen such that it targets the activating moiety to the tumor tissue to be treated with the drug

In some embodiments, the abnormal proliferation is of cancer cells.

In some embodiments, the cancer is selected from the group consisting of: breast cancer, colorectal cancer, diffuse large B-cell lymphoma, endometrial cancer, follicular lymphoma, gastric cancer, glioblastoma, head and neck cancer, hepatocellular cancer, lung cancer, melanoma, multiple myeloma, ovarian cancer, pancreatic cancer, prostate cancer, and renal cell carcinoma.

In some embodiments, the cancer that is treated is selected from the group consisting of: Acute myeloid leukemia (AML), Breast cancer, Chronic lymphocytic leukemia (CLL), Chronic myelogenous leukemia (CML), Hodgkin lymphoma, Multiple myeloma, Mycosis fungoides, Neuroblastoma, Non-Hodgkin lymphoma (NHL), and Ovarian cancer.

In some embodiments, the presently disclosed embodiments provide a compound for use in killing a cell. The compound is administered to the cell in an amount sufficient to kill said cell. In an exemplary embodiment, the compound is administered to a subject bearing the cell. In a further exemplary embodiment, the administration serves to retard or stop the growth of a tumor that includes the cell (e.g., the cell can be a tumor cell). For the administration to retard the growth, the rate of growth of the cell should be at least 10% less than the rate of growth before administration. Preferably, the rate of growth will be retarded at least 20%, 30%, 40%, 50%, 60%, 70%, 80%, 90%, or completely stopped.

Additionally, the presently disclosed embodiments provide a compound or a pharmaceutical composition of the presently disclosed embodiments for use as a medicament. The presently disclosed embodiments also provides a compound or a pharmaceutical composition for killing, inhibiting or delaying proliferation of a tumor or cancer cell, or for treating a disease wherein TLR7 and/or TLR8 are implicated.

### Dose and Administration

In some embodiments, the chemotherapeutic is provided in an amount that is capable of reducing the amount of MDCS in blood, spleen and/or tumor microenvironment by about 10, 20, 30, 40, 50, 60, 70, 80, 90, or 95% in the subject. Preferably is 30, 40, or 50% reduction.

In some embodiments, the chemotherapeutic is provided in an amount that is less than the amount when used as a monotherapy, such as about 10, 20, 30, 40, 50, 60, 70, 80, 90, or 95%, and preferably about 50%, of standard monotherapeutic dose.

In some embodiments, the chemotherapeutic is gemicitabine and the amount is 400-625 mg/m²

In some embodiments, the chemotherapeutics is administrated prior to the administration of the immunotherapy and is within 7 days prior to the administration of said immunotherapeutics.

In some embodiments, the chemotherapeutics is administrated prior to the administration of the immunotherapeutics.

In some embodiments, the chemotherapeutics is administrated at least one, two, three, four, five days, six day or seven days prior to the administration of said immunotherapeutics.

In some embodiments, the immunotherapeutics is administered after the amount of MDCS in blood, and/or tumor microenvironment is reduced by 10% to 95% in said subject after said administration of said chemotherapeutics.

In some embodiments, the immunotherapeutic is administrated in combination with an a targeted therapeutic against a cancer, either simultaneously, such as in a single formulation, or separately, such as in separate formulation.

In some embodiments “providing in an amount” comprises administering the indicated amount. In some embodiments “providing in an amount” comprises providing a dosage form, including but not limited to a unit dosage form, continuing the indicated amount.

### Effective Dosages

Pharmaceutical compositions suitable for use in the presently disclosed embodiments include compositions wherein the active ingredient is contained in a therapeutically effective amount, i.e., in an amount effective to achieve its intended purpose. The actual amount effective for a particular application will depend, inter alia, on the condition being treated.

For any compound described herein, the therapeutically effective amount can be initially determined from cell culture assays. Target plasma concentrations will be those concentrations of active compound(s) that are capable of inhibition cell growth or division. In preferred embodiments, the cellular activity is at least 25% inhibited. Target plasma concentrations of active compound(s) that are capable of inducing at least about 30%, 50%, 75%, or even 90% or higher inhibition of cellular activity are presently preferred. The percentage of inhibition of cellular activity in the patient can be monitored to assess the appropriateness of the plasma drug concentration achieved, and the dosage can be adjusted upwards or downwards to achieve the desired percentage of inhibition.

Therapeutically effective amounts for use in humans can also be determined from animal models. For example, a dose for humans can be formulated to achieve a circulating concentration that has been found to be effective in animals The dosage in humans can be adjusted by monitoring cellular inhibition and adjusting the dosage upwards or downwards, as described above.

A therapeutically effective dose can also be determined from human data for compounds which are known to exhibit similar pharmacological activities. The applied dose can be adjusted based on the relative bioavailability and potency of the administered compound as compared with the known compound.

Adjusting the dose to achieve maximal efficacy in humans can be accomplished based on the methods described above and other methods as are well-known in the art.

In some embodiments, a composition of the presently disclosed embodiments is delivered locally or regionally to a tumor located in the subject, delivered systemically, or delivered via intratumoral injection or by direct injection into tumor vasculature.

In some embodiments, the combination provided herein is formulated for systematic delivery. In some embodiments, a pharmaceutical composition is formulated for oral administration or parenteral injection. In some embodiments, a pharmaceutical composition is formulated for intravenous injection or intratumoral injection.

In another aspect, the presently disclosed embodiments provides a method for treating tumor or abnormal cell proliferation, in a subject that is in need of such treatment, comprising administering to the subject the combination provided herein.

In some embodiments, the method provided herein comprises administering to the subject an oral formulation comprising said immunotherapeutic in a dose of from about 0.0005 mg/kg, 0.0006 mg/mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, or 0.01 mg/kg, to about 0.02 mg/kg, all inclusive, twice per week.

In some embodiments, the method provided herein comprises administering to the subject an oral formulation comprising said immunotherapeutic in a dose of less than or about 0.0005mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, or 0.01 mg/kg, twice per week.

In some embodiments, the method provided herein comprises administering to the subject an intravenous formulation comprising said immunotherapeutic in a dose of from about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, or 0.006 mg/kg to about 0.015 mg/kg, all inclusive, weekly.

In some embodiments, the method provided herein comprises administering to the subject an intravenous formulation comprising said immunotherapeutic in a dose of about 40-50 mg/kg in divided dose over 2-5 days.

In some embodiments, the combination is administered repeatedly at intervals of 2, 3, or 4 weeks within each cycle.

In some embodiments, the method provided herein comprises administering to the subject an intravenous formulation comprising the chemotherapeutic in a dose of about 10 to 15 mg/kg, given every 7 to 10 days.

In some embodiments, the method provided herein comprises administering to the subject an intravenous formulation comprising the chemotherapeutic in a dose of about 3 to 5 mg/kg, twice weekly.

In some embodiments, the method provided herein comprises administering to the subject an intravenous formulation comprising the chemotherapeutic in a dose of about 60-120 mg/m²/day, continuous daily.

In some embodiments, the method provided herein comprises administering to the subject an oral formulation comprising the chemotherapeutics in a dose of about 400-1000 mg/m² divided over 4-5 days.

In some embodiments, the method provided herein comprises administering to the subject an intravenous formulation comprising the chemotherapeutic in a dose of about 50-100 mg/m²/day, or 1-5 mg/kg/day.

In some embodiments, the method provided herein comprises administering to the subject an intravenous formulation comprising the chemotherapeutic in the form of intermittent therapy, wherein 40-50 mg/kg is divided in doses administer over 2-5 days. The administration may be repeated at intervals of 2-4 week within each cycle. In some embodiments, the dose is 10 to 15 mg/kg given every 7 to 10 days; or 3 to 5 mg/kg twice weekly.

In some embodiments, the method provided herein comprises administering to the subject an intravenous formulation comprising the chemotherapeutic in the form of continuous therapy, with a dose of 1-2.5 mg/kg/day.

In some embodiments, the method provided herein comprises administering to the subject an oral formulation comprising the chemotherapeutic in the form of intermittent therapy, wherein a dose of 40-50 mg/kg is divided to be administer over 4-5 days.

In some embodiments, the method provided herein comprises administering to the subject an oral formulation comprising the chemotherapeutic with a dose of 1-5 mg/kg/day.

In the case of local administration, the systemic circulating concentration of administered compound will not be of particular importance. In such instances, the compound is administered so as to achieve a concentration at the local area effective to achieve the intended result.

Therapeutic amounts of specific antibodies disclosed herein can also be administered, as a component of the combination, with the immunotherapeutic, either in a single mixture form, or separately. In some embodiments, therapeutic amounts are amounts which eliminate or reduce the patient's tumor burden, or which prevent or reduce the proliferation of metastatic cells. The dosage will depend on many parameters, including the nature of the tumor, patient history, patient condition, the possible co-use of other oncolytic agents, and methods of administration. Methods of administration include injection (e.g., parenteral, subcutaneous, intravenous, intraperitoneal, etc.) for which the antibodies are provided in a nontoxic pharmaceutically acceptable carrier such as water, saline, Ringer's solution, dextrose solution, 5% human serum albumin, fixed oils, ethyl oleate, or liposomes. Typical dosages may range from about 0.01 to about 20 mg/kg, such as from about 0.1 to about 10 mg/kg. Other effective methods of administration and dosages may be determined by routine experimentation and are within the scope of this invention.

For other modes of administration, dosage amount and interval can be adjusted individually to provide plasma levels of the administered compound effective for the particular clinical indication being treated. For example, in one embodiment, a compound according to the presently disclosed embodiments can be administered in relatively high concentrations multiple times per day. Alternatively, it may be more desirable to administer a compound of the combination at minimal effective concentrations and to use a less frequent administration regimen. This will provide a therapeutic regimen that is commensurate with the severity of the individual's disease.

Utilizing the teachings provided herein, an effective therapeutic treatment regimen can be planned which does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

Toxicities and adverse events are sometimes graded according to a 5 point scale. A grade 1 or mild toxicity is asymptomatic or induces only mild symptoms; may be characterized by clinical or diagnostic observations only; and intervention is not indicated. A grade 2 or moderate toxicity may impair activities of daily living (such as preparing meals, shopping, managing money, using the telephone, etc.) but only minimal, local, or non-invasive interventions are indicated. Grade 3 toxicities are medically significant but not immediately life-threatening; hospitalization or prolongation of hospitalization is indicated; activities of daily living related to self-care (such as bathing, dressing and undressing, feeding oneself, using the toilet, taking medications, and not being bedridden) may be impaired. Grade 4 toxicities are life-threatening and urgent intervention is indicated. Grade 5 toxicity produces an adverse event-related death. Thus in various embodiments, use of a disclosed combination reduces the grade of a toxicity associated with treatment by at least one grade as compared to use of a similarly effective dosage the immunotherapeutic alone (if such effectiveness can be achieved at all). Alternatively the immunotherapeutic is has greater efficacy as part of the combination that can be achieved at the maximum tolerated dose of the immunotherapeutic used alone. In other embodiments use of a disclosed combination confines a toxicity to grade 2 or less, to grade 1 or less, or produces no observation of a particular toxicity.

While preferred embodiments of the presently disclosed embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention.

### EXAMPLES

The presently disclosed embodiments is further exemplified, but not limited, by the following and Examples that illustrate the preparation of compounds of the combination.

### Example 1

In vivo tumor cell killing using TLRL with PDL1 and Gemcitabine combination therapy in immune competent mice

Female 6-7-week-old C3H/HeN (C3H) mice were purchased from Japan SLC (Hamamatsu, Japan). All mice were maintained under specific pathogen-free conditions at Tokyo Medical and Dental University. All experimental procedures were reviewed and approved by the Animal Care and Use Committee of Tokyo Medical and Dental University (0170344A and A2018-262C). NR-S1 and SCC VII are murine squamous cell carcinoma (SCC) cell lines of C₃H origin. Cells were cultured for 5-7 days before using for tumor inoculation. NR-S1 (1×10⁶ cells) and SCCVII (2×10⁵ cells) were injected subcutaneously (s.c.) into the shaved right flank of syngeneic C3H mice, and tumor size was measured as described previously. Treatment was started when tumor volume in individual mice reached 30 mm³ (6-8 days after tumor inoculation). Resiquimod (1.7 μg/mouse) and/or anti-PD-L1 mAb (MIHS, rat IgG2a, 200 μg/mouse) were intraperitoneally administrated. Tumor diameters were measured along three orthogonal axes (x, y, and z) and tumor volume was calculated as (xyz)/2.

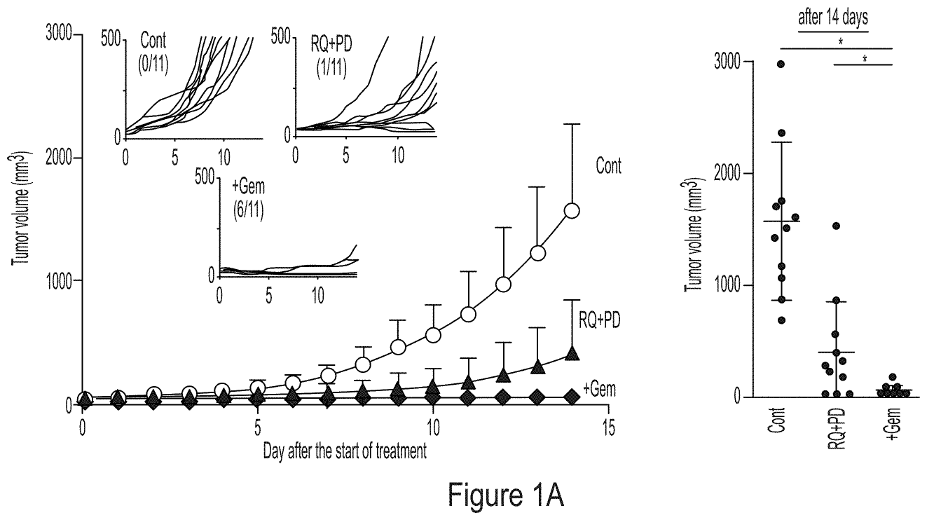

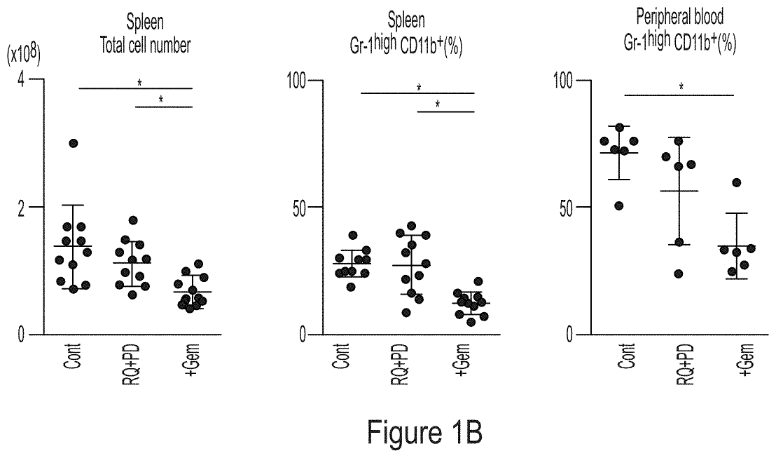

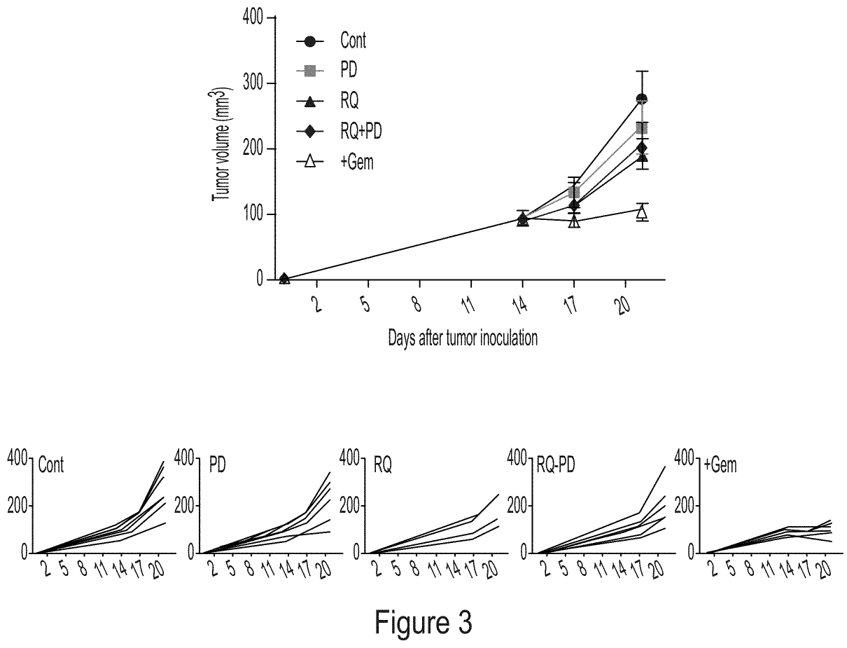

In two separate experiments, mice were treated with 1.7 μg of TLRL (resiquimod) or combination therapy with 200 μg of PDL1 antibody (clone: MIHS, rat IgG2Ga) every three days for 4 times in NR-S1 tumor model shown in FIG. 1 or weekly in 4T1 tumor model shown in FIG. 3. Gemcitabine (30 mg/kg) was intraperitoneally injected one day before the initial treatment.

### Example 2

Analysis of MSDCs in NR-S1 and SCCVII tumors

Tumor-infiltrating lymphocytes were isolated by enzyme digestion using collagenase I, hyaluronidase, and DNase and the density gradient centrifugation. Monoclonal antibodies (mAbs) against CD3 (17A2, rat IgG2b), CD4 (GK1.5, rat IgG2b), CD8 (53-6.72, rat IgG1), CD45 (30-F11, rat IgG2b), IFN-r (XMG1.2, rat IgG1), Foxp3 (FJK-165, rat IgG2a), CD11b (M1/70, rat IgG2b), Gr-1 (Ly6C/Ly6G, RB6-8C5, rat IgGa), Ly6C (HK1.4, rat IgG2a), Ly6G (1A8, rat IgG2a), F4/80 (BM8, rat IgG2a), CD86 (PO3.1, rat IgG2b), MHC class II (M5/114, rat IgG2b), and PD-L1 (MIHS, rat IgG2a) were used. All fluorochrome (FITC, PE, PE-Cyanine7, APC, APC-eFluor780, eFluor450, Brilliant Violet 510)-conjugated mAbs were obtained from Thermo Fisher Scientific (Carlsbad, Calif.), BD-Biosciences (San Jose, Calif.), or Biolegend (San Diego, Calif.). All stained cells were analyzed using a FACSVerse flow cytometer (BD Biosciences) and a FlowJo software (Tree Star, Ashland, Oreg.). Statistical analyses were performed using the Mann-Whitney U-test. A value of p <0.05 was considered to indicate significance.

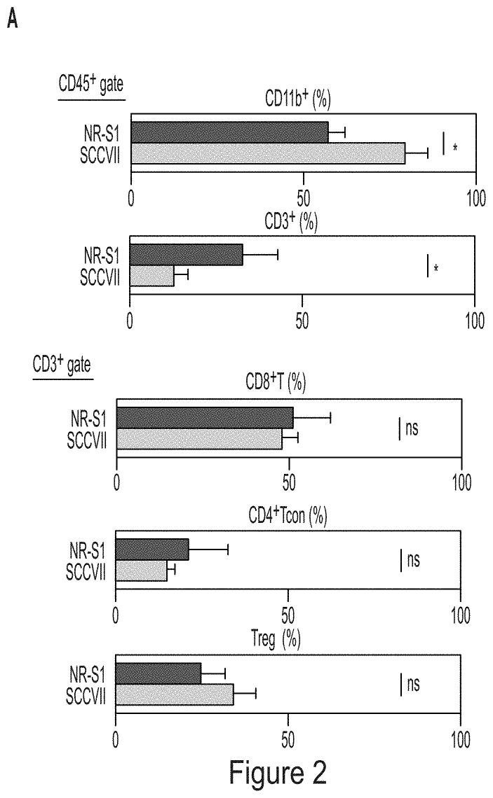

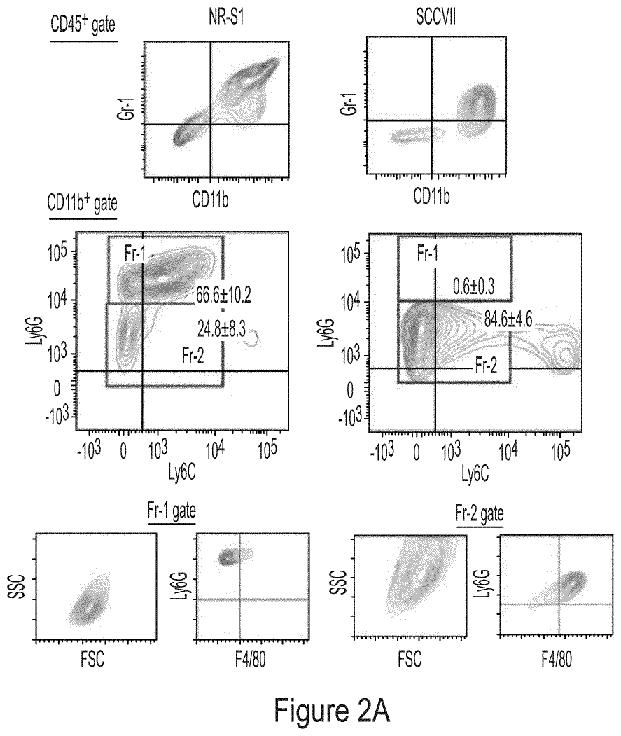

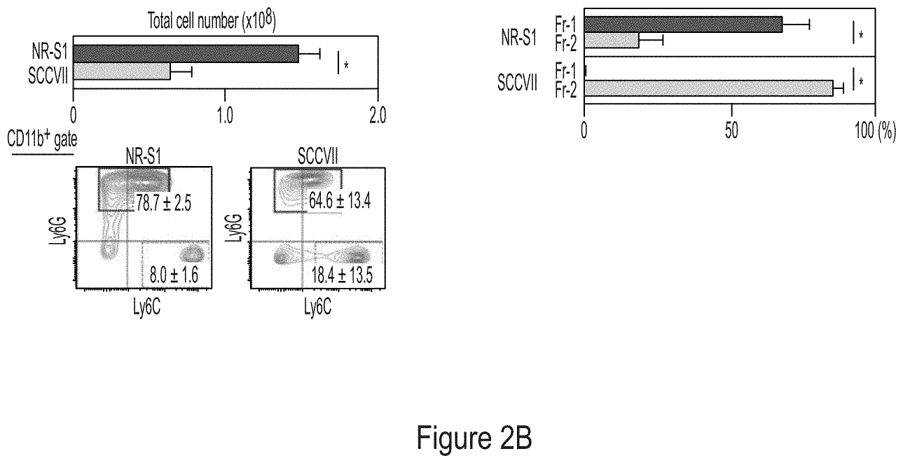

The results depicted in FIG. 2 demonstrated that the immune status of the tumor-associated macrophages of the two SCC tumors are different, particularly in terms of CD11b+myeloid cells. Ly6GhighLy6C-F4/80-tumor-associated neutrophils predominated in NR-S1 tumors, and Ly6GlowLy6Clow/-F4/80+ tumor-associated macrophages predominated in SCCVII tumors.

The proportion of CD11b+ cells in NR-S1 tumors was significantly lower than that in SCCVII tumors, and the proportion of CD3+ T cells was higher (FIG. 2A).

CD11b+ cells showed different levels of Gr-1 expression (FIG. 2B, upper right). A major fraction of the CD11b+ cells in NR-S1 tumors had a Ly6GhighLy6C-phenotype (Fr-1), whereas the majority of CD11b+ cells in SCCVII tumors had a Ly6GlowLy6C-phenotype (Fr-2). SCCVII Fr-2 cells were FSChigh, SSChigh, and F4/80+.

The expression of F4/80 and Ly6G, cell size by FSC, and intracellular granule status by SSC indicated that Fr-1 and Fr-2 are tumor-associated neutrophils and tumor-associated macrophage, respectively.

Spleens from NR-S1 tumor-bearing mice showed apparent splenomegaly and a twofold greater expression profiles of Ly6C and Ly6G (FIG. 2B).

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described

What is claimed is:

1. A method for treating tumor or abnormal cell proliferation, in a subject that is in need of such treatment, comprising administering to said subject:
a chemotherapeutic in an amount that is capable of reducing myeloid-derived suppressor cell (MDSC) population in blood, spleen, and/or tumor microenvironment in said subject; and
(ii) an effective amount of an immunotherapeutic.

2. The method of claim 1, wherein said MDSC inhibitor is selected from the group consisting of: Paclitaxel, Gemcitabine, 5-Fluorouracile, Oxaliplatin, Cisplatin, Carboplatin, Dasatinib, Sunitinib, and Doxorubicin.

3. The method of claim 1, wherein said chemotherapeutic is of an amount that is capable of reducing the amount of MDCS in blood, spleen and/or tumor microenvironment by 10% to 95% in said subject.

4. The method of claim 1, wherein said chemotherapeutic is of an amount that is less than the amount when used as a monotherapy.

5. The method of claim 1, where said chemotherapeutic is gemicitabine and the amount is 400-625 mg/m².

6. The method of claim 1, wherein said MDSC expresses CD11b, CD15, CD33 and CD66b.

7. The method of claim 1, wherein said MDSC expresses CD11b, CD14, and CD33.

8. The method of claim 1, wherein said chemotherapeutic is administrated prior to the administration of said immunotherapeutic and is within 7 days prior to the administration of said immunotherapeutic.

9. The method of claim 1, wherein said chemotherapeutic is administrated prior to the administration of said immunotherapeutic.

10. The method of claim 1, wherein said chemotherapeutic is administrated at least one, two, three, four, five days, six day or seven days prior to the administration of said immunotherapeutic.

11. The method of claim 1, wherein said immunotherapeutic is administrated after the amount of MDCS in blood, and/or tumor microenvironment is reduced by 10% to 95% in said subject after said administration of said chemotherapeutic.

12. The method of claim 1, wherein said immunotherapeutic comprising a TLR7 and/or TLR8 agonist.

13. The method of claim 1, wherein said immunotherapeutic that is an agonist for both TLR7 and TLR8.

14. The method of claim 1, where said immunotherapeutic has a structure of Formula (I): 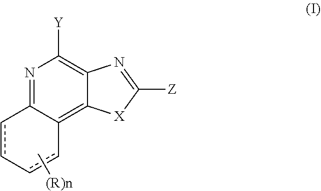
wherein dashed line represents bond or absence of bond;
X is S or —NR₁, R₁ is —W₀—W₁—W₂—W₃—W₄,
W₀ is a bond, alkyl, alkenyl, alkynyl, alkoxy, or alkyl-S-alkyl,
W₁ is a bond, —O—, or —NR₂, wherein R₂ is hydrogen, alkyl or alkenyl,
W₂ is a bond, —O—, —C(O)—, —C(S)—, or —S(O)₂—,
W₃ is a bond, —NR₃—, wherein R₃ is hydrogen, alkyl or alkenyl,
W₄ is hydrogen, alkyl, alkenyl, alkynyl, alkoxy, cycloalkyl, aryl, aryloxy, heteroaryl, or heterocyclyl, each of which is optionally substituted by one or more substituents selected from the group consisting of hydroxyl, alkoxy, alkyl, alkenyl, alkynyl, cycloalkyl, aryl, heteroaryl, heterocyclyl, —NH₂, nitro, alkyl-hydroxyl, alkyl-aryl, alkyl-heteroaryl, -alkyl-heterocyclyl, —O—R₄, —O-alkyl-R₄, -alkyl-O—R₄, —C(O)—R₄, -alkyl-C(O)—R₄, -alkyl-C(O)—O—R₄, —C(O)—O—R₄, —S—R₄, —S(O)₂—R₄, —NH—S(O)₂—R₄, -alkyl-S—R₄, -alkyl-S(O)₂—R₄, —NHR₄, —NR₄R₄, —NH-alkyl-R₄, halogen, —CN, —NO₂, and —SH, wherein R₄ is independently hydrogen, alkyl, alkenyl, -alkyl-hydroxyl, aryl, heteroaryl, heterocyclyl, or haloalkyl;
Z is hydrogen, alkyl, alkenyl, alkynyl, alkoxy, aryl, haloalkyl, heteroaryl, heterocyclyl, each of which can be optionally substituted by one or more substituents selected from the group consisting of hydroxyl, alkoxy, alkyl, alkenyl, alkynyl, aryl, heteroaryl, heterocyclyl, halogen, cyano, nitro, —N(R₅)₂, -alkoxy-alkyl, -alkoxy-alkenyl, —C(O)-alkyl, —C(O)—O-alkyl, —O—C(O)-alkyl, —C(O)—N(R₅)₂, aryl, heteroaryl, —CO-aryl, and -CO-heteroaryl, wherein each R₅ is independently hydrogen, alkyl, haloalkyl, -alkyl-aryl, or -alkyl-heteroaryl;
R is hydrogen, alkyl, alkoxy, haloalkyl, halogen, aryl, heteroaryl, heterocyclyl, each of which is optionally substituted by one or more substituents selected from the group consisting of hydroxyl, alkoxy, alkyl, alkenyl, alkynyl, cycloalkyl, aryl, heteroaryl, heterocyclyl, —NH₂, nitro, -alkyl-hydroxyl, -alkyl-aryl, -alkyl-heteroaryl, -alkyl-heterocyclyl, —O—R₄, —O-alkyl-R₄, -alkyl-O—R₄, —C(O)—R₄, —C(O)—NH—R₄, —C(O)—NR₄R₄, -alkyl-C(O)—R₄, -alkyl-C(O)—O—R₄, —C(O)—O—R₄, —O—C(O)—R₄, —S—R₄, —C(O)—S—R₄, —S—C(O)—R₄, —S(O)₂—R₄, —NH—S(O)₂—R₄, -alkyl-S—R₄, -alkyl-S(O)₂—R₄, —NHR₄, —NR₄R₄, —NH-alkyl-R₄, halogen, —CN, and —SH, wherein R₄ is independently hydrogen, alkyl, alkenyl, alkoxy, -alkyl-hydroxyl, aryl, heteroaryl, heterocyclyl, or haloalkyl;
n is 0, 1, 2, 3, or 4;
Y is —NR₆R₇, —CR₆R₇R₈, or -alkyl-NH₂, each of which can be optionally substituted by one or more substituents selected from the group consisting of hydroxyl, alkoxy, alkyl, alkenyl, alkynyl, —NH₂, halogen, —N(R₅)₂, -alkoxy-alkyl, -alkoxy-alkenyl, —C(O)-alkyl, —C(O)—O-alkyl, —C(O)—N(R₅)₂, aryl, heteroaryl, —CO-aryl, and —CO-heteroaryl,
wherein R₆, R₇ and R₈ are independently hydrogen, alkyl, alkenyl, alkoxy, alkylamino, dialkylamino, alkylthio, arylthio, -alkyl-hydroxyl, -alkyl-C(O)—O—R₉, -alkyl-C(O)—R₉, or -alkyl-O—C(O)—R₉, wherein each R₅ is independently hydrogen, alkyl, haloalkyl, -alkyl-aryl, or -alkyl-heteroaryl, wherein R₉ is hydrogen, alkyl, alkenyl, halogen, or haloalkyl;
X and Z taken together may optionally form a (5-9)-membered ring.

15. The method of claim 1, wherein said immunotherapeutic is a compound selected from the group consisting of:
2-propylthiazolo[4,5-c]quinolin-4-amine,
1-(2-methylpropyl)-1H-imidazo[4,5-c]quinolin-4-amine,
4-amino-2-(ethoxymethyl)-a,a-di-methyl-1H-imidazo[4,5-c]quinoline-1-ethanol,
1-(4-amino-2-ethylaminomethylimidazo-[4,5-c]quinolin-1-yl)-2-methylpropan-2-ol,
N-[4-(4-amino-2-ethyl-1H-imidazo[4,5-c]quinolin-1-yl)butyl-]methanesulfonamide,
4-amino-2-ethoxymethyl-a,a-dimethyl-6,7,8,9-tetrahydro-1h-imidazo[4,5-c]quinoline-1-ethanol,
4-amino-aa-dimethyl-2-methoxyethyl-1H-imidazo[4,5-c]quinoline-1-ethanol,
1-{2-[3-(benzyloxy)propoxy]ethyl}-2-(ethoxymethyl)-1H-imidazo[4,5-c]quinolin-4-amine,
N-[4-(4-amino-2-butyl-1H-imidazo[4,5-c][1,5]naphthyridin-1-yl)butyl]-n′-butylurea,
N1-[2-(4-amino-2-butyl-1H-imidazo[4,5-c][1,5]naphthyridin-1-yl)ethyl]-2-amino-4-methylpentanamide,
N-(2-{2-[4-amino-2-(2-methoxyethyl)-1H-imidazo[4,5-c]quinolin-1-yl]ethoxy}ethyl)-n′-phenylurea,
1-(2-amino-2-methylpropyl)-2-(ethoxymethyl)-1H-imidazo[4,5-c]quinolin-4-amine,
1-{4-[(3,5-dichlorophenyl)sulfonyl]butyl}-2-ethyl-1H-imidazo[4,5-c]quinolin-4-amine,
N-(2-{2-[4-amino-2-(ethoxymethyl)-1H-imidazo[4,5-c]quinolin-1-yl]ethoxy}ethyl)-n′-cyclohexylurea,
N-{3-[4-amino-2-(ethoxymethyl)-1H-imidazo[4,5-c]quinolin-1-yl]propyl}-n′-(3-cyanophenyl)thiourea,
N-[3-(4-amino-2-butyl-1H-imidazo[4,5-c]quinolin-1-yl)-2,2-dimethylpropyl]benzamide,
2-butyl-1-[3-(methylsulfonyl)propyl]-1H-imidazo[4,5-c]quinolin-4-amine,
N-{2-[4-amino-2-(ethoxymethyl)-1H-imidazo[4,5-c]quinolin-1-yl]-1,1-dimethylethyl}-2-ethoxyacetamide,
1-[4-amino-2-ethoxymethyl-7-(pyridin-4-yl)-1H-imidazo[4,5-c]quinolin-1-yl]-2-methylpropan-2-ol,
1-[4-amino-2-(ethoxymethyl)-7-(pyridin-3-yl)-1H-imidazo[4,5-c]quinolin-1-yl]-2-methylpropan-2-ol,
N-{3-[4-amino-1-(2-hydroxy-2-methylpropyl)-2-(methoxyethyl)-1H-imidazo[4,5-c]quinolin-7-yl]phenyl}methanesulfonamide,
1-[4-amino-7-(5-hydroxymethylpyridin-3-yl)-2-(2-methoxyethyl)-1 H-imidazo[4,5-c]quinolin-1-yl]-2-methylpropan-2-ol,
3-[4-amino-2-(ethoxymethyl)-7-(pyridin-3-yl)-1H-imidazo[4,5-c]quinolin-1-yl]propane-1,2-diol,
1-[2-(4-amino-2-ethoxymethyl-1H-imidazo[4,5-c]quinolin-1-yl)-1,1-dimethylethyl]-3-propylurea,
1-[2-(4-amino-2-ethoxymethyl-1H-imidazo[4,5-c]quinolin-1-yl)-1,1-dimethylethyl]-3-cyclopentylurea,
1-[(2,2-dimethyl-1,3-dioxolan-4-yl)methyl]-2-(ethoxymethyl)-7-(4-hydroxymethylphenyl)-1H-imidazo[4,5-c]quinolin-4-amine,
4-[4-amino-2-ethoxymethyl-1-(2-hydroxy-2-methylpropyl)-1H-imidazo[4,5-c]quinolin-7-yl]-N-methoxy-N-methylbenzamide,
2-ethoxymethyl-N1-isopropyl-6,7,8,9-tetrahydro-1H-imidazo[4,5-c]quinoline-1,4-diamine,
1-[4-amino-2-ethyl-7-(pyridin-4-yl)-1H-imidazo[4,5-c]quinolin-1-yl]-2-methylpropan-2-ol,
N-[4-(4-amino-2-ethyl-1 H-imidazo[4,5-c]quinolin-1-yl)butyl]methanesulfonamide, and
N-[4-(4-amino-2-butyl-1 H-imidazo[4,5-c][1,5]naphthyridin-1-yl)butyl]-n′-cyclohexylurea.

16. The method of claim 1, where said immunotherapeutic comprises resiquimod.

17. The method of claim 1, wherein said immunotherapeutic is of an amount that is capable of:
(1) inducing IFN-α production by human blood DCs;
(2) inducing TNF-α production by human blood DCs; and/or
(3) inducing IL-12-α production by human blood DCs.

18. The method of claim 1, wherein said immunotherapeutic is of an amount capable of activating a human plasmacytoid dendritic cell, myeloid dendritic cell, or NK cell, or a combination thereof.

19. The method of claim 1, wherein said immunotherapeutic is administrated in combination with a targeted therapeutic against a cancer.

20. The method of claim 19, wherein said targeted therapeutic is capable of binding to a tumor cell specifically or preferably in comparison to a non-tumor cell.

21. The method of claim 19, wherein said tumor cell is of a carcinoma, a sarcoma, a lymphoma, a myeloma, or a central nervous system cancer.

22. The method of claim 19, wherein said targeted therapeutic is capable of binding to a tumor antigen specifically or preferably in comparison to a non-tumor antigen.

23. The method of claim 22, wherein said tumor antigen is selected from the group consisting of: CD2, CD19, CD20, CD22, CD27, CD33, CD37, CD38, CD40, CD44, CD47, CD52, CD56, CD70, CD79, and CD137.

24. The method of claim 22, wherein said tumor antigen is selected from the group consisting of: 4-1BB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin αν, Integrin ανβ, KIR, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2,VEGFR-1, VEGFR-2, VEGFR-3, and variants thereof.

25. The method of claim 19, wherein said targeted therapeutic comprises an immunoglobulin, a protein, a peptide, a small molecule, a nanoparticle, or a nucleic acid.

26. The method of claim 19, wherein said targeted therapeutic comprises an antibody, or a functional fragment thereof.

27. The method of claim 26, wherein said antibody is selected from the group consisting of: Rituxan (rituximab), Herceptin (trastuzumab), Erbitux (cetuximab), Vectibix (Panitumumab), Arzerra (Ofatumumab), Benlysta (belimumab), Yervoy (ipilimumab), Perjeta (Pertuzumab), Tremelimumab, Nivolumab, Dacetuzumab, Urelumab, MPDL3280A, Lambrolizumab, Blinatumomab, CT-011, MK-3475, BMS-936559, MED14736, MSB0010718C, and margetuximab (MGAH22).

28. The method of claim 26, wherein said targeted therapeutic comprises a Fab, Fab′, F(ab′)2, single domain antibody, T and Abs dimer, Fv, scFv, dsFv, ds-scFv, Fd, linear antibody, minibody, diabody, bispecific antibody fragment, bibody, tribody, sc-diabody, kappa (lamda) body, BiTE, DVD-Ig, SIP, SMIP, DART, or an antibody analogue comprising one or more CDRs.

29. The method of claim 19, wherein said targeted therapeutic comprises a ATWLPPR (SEQ ID NO:1) polypeptide of VEGFR, Thrombospondin-1 mimetics, CDCRGDCFCG (SEQ ID NO:2) (cyclic) polypeptide, SCH 221153 fragment, NCNGRC SEQ ID NO:3) (cyclic) polypeptide, CTTHWGFTLC (SEQ ID NO:4) polypeptide, CGNKRTRGC (SEQ ID NO:5) polypeptide (LyP-1), Octreotide, Vapreotide, Lanreotide, C-3940 polypeptide, Decapeptyl, Lupron, Zoladex, or Cetrorelix.

30. The method of claim 19, wherein said targeted therapeutic comprises extracellular domains (ECD) or soluble form of PD-1, PDL-1, CTLA4, BTLA, KIR, TIM3, 4-1BB, LAG3, full length of partial of a surface ligand amphiregulin, betacellulin, EGF, ephrin, epigen, epiregulin, IGF, neuregulin, TGF, TRAIL, or VEGF.

31. The method of claim 1, wherein said immunotherapeutic is delivered systemically.

32. The method of claim 1, wherein said immunotherapeutic is administered by oral administration or parenteral injection.

33. The method of claim 1, wherein said immunotherapeutic is administrated by intravenous injection or intratumoral injection.

34. The method of claim 1, wherein said abnormal cell proliferation comprises a pre-cancerous lesion.

35. The method of claim 1, wherein said abnormal proliferation is of cancer cells.

36. The method of claim 35, wherein said cancer is selected from the group consisting of: Acute myeloid leukemia (AML), Breast cancer, Chronic lymphocytic leukemia (CLL), Chronic myelogenous leukemia (CML), Hodgkin lymphoma, Multiple myeloma, Mycosis fungoides, Neuroblastoma, Non-Hodgkin lymphoma (NHL), Ovarian cancer, and Retinoblastoma.

37. The method of claim 1, comprising administering to said subject an oral formulation comprising said immunotherapeutic in a dose of from about 0.0005 mg/kg, 0.0006 mg/mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, or 0.01 mg/kg, to about 0.02 mg/kg, all inclusive, twice per week.

38. The method of claim 1, comprising administering to said subject an oral formulation comprising said immunotherapeutic in a dose of from 0.0001 mg/kg to less than or about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, or 0.01 mg/kg, twice per week.

39. The method of claim 1, comprising administering to said subject an intravenous formulation comprising said immunotherapeutic in a dose of from about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, or 0.006 mg/kg to about 0.015 mg/kg, all inclusive, weekly.

40. The method of claim 1, comprising administering to said subject an intravenous formulation comprising said immunotherapeutic in a dose of from 0.0001mg/kg to less than or about 0.003 mg/kg, 0.004 mg/kg, 0.005 mg/kg, 0.006 mg/kg, or 0.01 mg/kg, weekly.

41. The method of claim 18 wherein said immunotherapeutic in said subject has a local concentration that is between about 0.005 μg/ml and about 12 μg/ml.

42. The method of claim 1, wherein said immunotherapeutic in said subject has a local concentration that is between about 0.05 μg/ml, 0.1 μg/ml, 0.15 μg/ml, 0.2 μg/ml, 0.3 μg/ml, or 0.4 μg/ml, and about 0.5 μg/ml.

43. The method of claim 1, comprising administering to said subject an intravenous formulation comprising said chemotherapeutic in a dose of about 40-50 mg/kg in divided dose over 2-5 days.

44. The method of claim 2, wherein said immunotherapeutic is administered repeatedly at intervals of 2-4 weeks.

45. The method of claim 1, comprising administering to said subject an intravenous formulation comprising said chemotherapeutic in a dose of about 10 to 15 mg/kg, given every 7 to 10 days.

46. The method of claim 1, comprising administering to said subject an intravenous formulation comprising said chemotherapeutic in a dose of about 3 to 5 mg/kg, twice weekly.

47. The method of claim 1, comprising administering to said subject an intravenous formulation comprising said chemotherapeutic in a dose of about 60-120 mg/m²/day, daily.

48. The method of claim 1, comprising administering to said subject an oral formulation comprising said chemotherapeutic in a dose of about 400-1000 mg/m² divided over 4-5 days.

49. The method of claim 1, comprising administering to said subject an intravenous formulation comprising said chemotherapeutic in a dose of about 50-100 mg/m²/day, or 1-5 mg/kg/day.


SEQUENCE LISTING (5 sequences)

SEQ ID NO: 1 - protein, 7 aa, Artificial Sequence
  VEGFR polypeptide
ATWLPPR

SEQ ID NO: 2 - protein, 10 aa, Artificial Sequence
  targeted therapeutic polypeptide
CDCRGDCFCG

SEQ ID NO: 3 - protein, 6 aa, Artificial Sequence
  targeted therapeutic polypeptide
NCNGRC

SEQ ID NO: 4 - protein, 10 aa, Artificial Sequence
  targeted therapeutic polypeptide
CTTHWGFTLC

SEQ ID NO: 5 - protein, 9 aa, Artificial Sequence
  LyP-1 polypeptide
CGNKRTRGC